(19) United States
(12) Patent Application Publication
(10) Pub. No.: US 2017/0246235 A1
(43) Pub. Date: Aug. 31, 2017

(54) GREEN TEA COMPOSITIONS

(71) Applicant: Plandai Biotechnology Inc., Goodyear, AZ (US)

(72) Inventor: Roger DUFFIELD, London (GB)

(21) Appl. No.: 15/508,450

(22) PCT Filed: Sep. 2, 2015
(86) PCT No.: PCT/US2015/048190

§ 371 Date: Mar. 2, 2017

Related U.S. Application Data
(60) Provisional application No. 62/045,419, filed on Sep. 3, 2014.
(60) Provisional application No. 62/077,634, filed on Nov. 10, 2014.
(60) Provisional application No. 62/117,390, filed on Feb. 17, 2015.
(60) Provisional application No. 62/173,489, filed on Jun. 10, 2015.

(51) Int. Cl. A61K 36/82; A61K 31/353; A61K 47/14; A61K 47/10; A61K 47/12; A61K 47/44; A23F 3/18; A61K 9/00

(52) U.S. Cl. CPC A61K 36/82; A23F 3/18; A61K 31/353; A61K 9/0014; A61K 9/0053; A61K 47/10; A61K 47/12; A61K 47/44; A61K 47/14

(57) ABSTRACT

Disclosed herein are green tea plant material extracts, and methods of using the extracts, comprising at least one green tea catechin selected from the group consisting of epi-gallocatechin, catechin, epicatechin, epigallocatechin-3-gallate, gallocatechin gallate, epicatechin-3-gallate, catechin gallate, and gallocatechin, wherein the compositions of the disclosure provide greater bioavailability of at least one green tea catechin.

### FIELD OF THE DISCLOSURE

Green tea-based compositions for nutritional, pharmaceutical, and anti-aging uses are disclosed herein.

### BACKGROUND OF THE DISCLOSURE

Tea is one of the world's most widely consumed plants. Tea has been prepared as a beverage for five thousand years, and tea's medicinal uses and therapeutic potential date back at least two thousand years. For most of that time, tea was consumed as a drink made from fresh leaves or powdered leaves, and the liquid contained the full range of nutrients, micronutrients, and antioxidants that are now being studied as beneficial components. In particular, catechins are among the polyphenolic compounds found in tea, especially green tea. These polyphenols have been a focus of study for many years in relation to a wide range of health issues.

The potential for green tea to have a role in current medicine is documented by reports of its use for treating conditions including malaria, HIV, obesity, diabetes, and cancer. Despite early promising reports, it is not yet widely used for these or other medicinal uses. Reasons for the current lack of clinical use of green tea catechins include low bioavailability and low bioaccessibility, as recognized by experts in this field.

For example, in 2007, Ming Hu published an editorial entitled “Commentary: Bioavailability of Flavenoids and Polyphenols: A Call to Arms,” Mol. Pharm. 4:803-806 (2007). Included in the commentary are theaflavins from black tea and EGCG from green tea. Dr. Hu noted that (a) the poor bioavailability of polyphenols leads to large exposure differences among the clinical trial participants, and (b) few government-sponsored clinical trials are conducted on dietary polyphenols because of the limited, or non-existent, intellectual property position on these agents. Dr. Hu concluded that there is an urgent need to increase bioavailability of polyphenols, in part so that smaller populations can be used to conduct clinical trials of polyphenol use in human medicine.

This conclusion is consistent with that of S. Wolfram (J. Am. Coll. Nutr. 26:373S-388S) in 2004, who stated in reference to green tea and health effects, “To prove the effectiveness for disease prevention or treatment, several multi-center, long-term clinical studies investigating the effects of one precisely-defined green tea product on cardiovascular and metabolic endpoints would be necessary.”

Dietary green tea consumption is reputed to correlate with lower cancer incidence and mortality among certain populations. Several mechanisms have been proposed, including induction of apoptotic cell death and cell cycle arrest in tumor cells but not normal cells. One study suggests that the proteasome-mediated degradation pathway may be a target for green tea polyphenols, EGCG in particular. However, the authors of that study also indicate that the data are inconsistent, and conclude that there is a need for “more potent, stable and specific polyphenol proteasome inhibitors as novel anti-cancer agents.” (Dou, Q. P. et al., Inflammopharmacology 16:208-212, 2008.)

A green tea catechin product on the market and available to the public is Teavigo™. According to the manufacturer, Teavigo™ is prepared by first extracting green tea leaves with water, followed by extraction with ethyl acetate. This extract is concentrated and spray-dried to yield the extract in powder form. The final product is produced by adsorption chromatography of the extract; the eluate is then concentrated, crystallized, dried, and placed in containers.

The manufacturer states that Teavigo™ contains over 90% EGCG and 5% or less of other catechins. Thus, one potential disadvantage from a clinical perspective is the comparative reduction in catechins that other studies have shown to be synergistic when administered with EGCG. Teavigo™ does not appear to meet the need for a more bioavailable green tea catechin preparation.

In 2012, green tea was still being described as having a “potential role.” Mak, J. C. (Clin. Exp. Pharmacol. Physiol. 39:265-273) reviewed the studies on green tea catechins for a variety of disease therapies and noted, “the optimal dose has not yet been established to enable any solid conclusions to be drawn regarding the various health benefits of green tea or its constituents in humans.” Mak also noted that the in vitro data for EGCG was performed using EGCG concentrations in the micromolar range, which the author deemed “physiologically irrelevant.”

There is an urgent unmet need for effective prevention and treatment of many conditions and diseases caused by human and animal pathogens. There is also a need for improved methods and materials for controlling and reducing inflammation, which is associated with numerous chronic illnesses and signs of aging in humans, and for preventing and controlling obesity, diabetes, and cancer. This application helps fulfill these and other needs by providing compositions derived from and/or based on green tea plant material, and methods of using these compositions.

### SUMMARY OF THE DISCLOSURE

Provided is a green tea extract having improved bioavailability in mammals, including humans, wherein the extract is in the form of amorphous crystalline structure.

Also provided is an amorphous crystalline form of green tea extract, containing at least one catechin selected from the group consisting of gallocatechin (GC), epi-gallocatechin (EGC), catechin (C), epicatechin (EC), gallocatechin gallate (GCG), epigallocatechin-3-gallate (EGCG), catechin gallate (CG), and epicatechin-3-gallate (ECG).

Provided is a composition comprising at least one green tea catechin having anti-aging activity in a mammal, the composition comprising extract of green tea plant material wherein the cis/trans ratio of the at least one catechin in the extract is equivalent to the cis/trans ratio in the plant.

Further provided is a composition comprising at least one green tea catechin for treating or preventing obesity in a mammal.

Also provided is a composition comprising at least one green tea catechin having increased bioavailability in a mammal, the composition comprising at least one catechin selected from the group consisting of gallocatechin (GC), epi-gallocatechin (EGC), catechin (C), epicatechin (EC), gallocatechin gallate (GCG), epigallocatechin-3-gallate (EGCG), catechin gallate (CG), and epicatechin-3-gallate (ECG), wherein the catechin(s) exhibit increased bioavailability. The composition can contain two, three, four, five, six, seven, or eight of the catechins GC, EGC, C, EC, GCG, EGCG, CG, and ECG.

Provided is a composition comprising epi-gallocatechin (EGC), epicatechin (EC), epigallocatechin-3-gallate (EGCG), and epicatechin-3-gallate (ECG), wherein the cis/trans ratio of these catechins is equivalent to the cis/trans ratio in the green tea plant material.

In the compositions, the cis/trans ratio can be 95/5 cis/trans.

Further provided is a composition of catechins from plant material consisting of green tea leaves, wherein the green tea leaves are from the plant Camellia sinensis.

Also provided is a composition comprising at least one green tea catechin for treating or preventing malaria infection in a mammal, the composition comprising extract of green tea plant material wherein the bioavailability of at least one catechin in the extract is increased.

Further provides are methods of using the compositions.

### BRIEF DESCRIPTION OF THE DRAWINGS

FIG. 1 is a diagram showing the bioactivity of green tea catechins.

FIG. 2 shows the chemical structure of major green tea catechins, including epicatechin (EC), epi-gallocatechin (EGC), epicatechin-3-gallate (ECG) and epigallocatechin-3-gallate (EGCG).

FIG. 3 is a graph showing the decrease in P. falciparum NF54 strain survival after culture in the presence of Phytofare® catechin complex (GTWL 1). The IC₅₀-values were obtained using a non-linear dose-response curve fitting analysis via Graph Pad Prism v4.0 software.

FIG. 4 has top and bottom graphs showing the effect of green tea extract on P. falciparum. Growth was measured by hypoxanthine uptake using radiolabeled hypoxanthine. The top graph of FIG. 4 shows three independent IC₅₀ assays for the green tea extract. () indicates rep. 1 IC₅₀ 67.7 μM; (▪) indicates rep. 2 IC₅₀ 112.2 μM; and (♦) indicates rep. 3 IC₅₀ 67.2 μM. The bottom graph of FIG. 4 shows the means of the three independent experiments with error bars indicating S.E.M. IC₅₀ is 80.78 μM±1.11. Graphpad Prism 6 software was used to analyse the data using the equation for log(inhibitor) vs. normalized response (variable slope).

FIG. 5 has top and bottom graphs showing the effect of caffeine on P. falciparum, as controls for the experiments shown in FIG. 4. The top graph of FIG. 5 shows three independent IC₅₀ assays for caffeine. () rep. 1, (▪) rep. 2, and (♦) rep 3 had no calculable IC₅₀ values. The bottom graph of FIG. 5 shows the means of the three independent experiments with error bars indicating S.E.M. Graphpad Prism 6 software was used to analyse the data using the equation for log(inhibitor) vs. normalized response (variable slope).

FIG. 6 is a time concentration curve for epigallocatechin for Example 44.

FIG. 7 is a time concentration curve for gallocatechin gallate for Example 44.

FIG. 8 is a time concentration curve for epicatechin for Example 44.

FIG. 9 is a time concentration curve for epicatechin gallate for Example 44.

FIG. 10 is a time concentration curve for gallocatechin for Example 44.

FIG. 11 is a time concentration curve for epigallocatechin gallate for Example 44.

FIG. 12 is a time concentration curve for catechin for Example 44.

FIG. 13 is a time concentration curve for catechin gallate for Example 44.

FIG. 14 is a total catechins plasma concentration curve for Example 44.

FIG. 15 shows enhancement in bioavailability by use of Phytofare® compositions for Example 44. Open bars represent AUC (area under the curve) enhancement, Phytofare®/comparator. Closed bars represent Cₘₐₓ enhancement, Phytofare®/comparator.

FIG. 16 is a table showing the stability of catechins in oral dosage form. Top table, Comparator, total catechins. Bottom table, Phytofare®, total catechins.

FIG. 17 depicts stability of total catechins at different conditions, comparing the Comparator with Phytofare®.

FIG. 18 is a table showing a summary of averages of catechin plasma levels of 27 participants in Arm 1 (Comparator) and Arm 2 (Phytofare®) of the bioavailability study described in Example 44.

FIG. 19 shows the comparative peak average concentrations (Cₘₐₓ) of catechins attained in the plasma of participants after oral administration of the commercial and Phytofare® extracts.

FIG. 20 shows the comparative areas under the curve (AUC) calculated for the catechins after oral administration of commercial and Phytofare® products, using Prism Graphpad after normalization of the data.

FIG. 21 shows a comparison of the average times after oral administration of the commercial and Phytofare® extract to reach the peak catechin concentrations.

FIGS. 22A-22C show three tables with catechin levels as results of Arm 1, Arm 2 and Arm 3 oral dosing of green tea compositions as described in Example 44. The three compositions were generic green tea extract (Arm 1), a Phytofare® Catechin Complex (Arm 2), and a Phytofare® Pheroid®Catechin Complex (Arm 3). FIG. 22A shows Arm 1; FIG. 22B shows Arm 2; FIG. 22C shows Arm 3.

FIG. 23 is a confocal micrograph of catechin extract sample processed using the method of Example 1.

FIGS. 24A-24I show the Preclinical Drug Development Platform Pheroid® Phytofare® Technology.

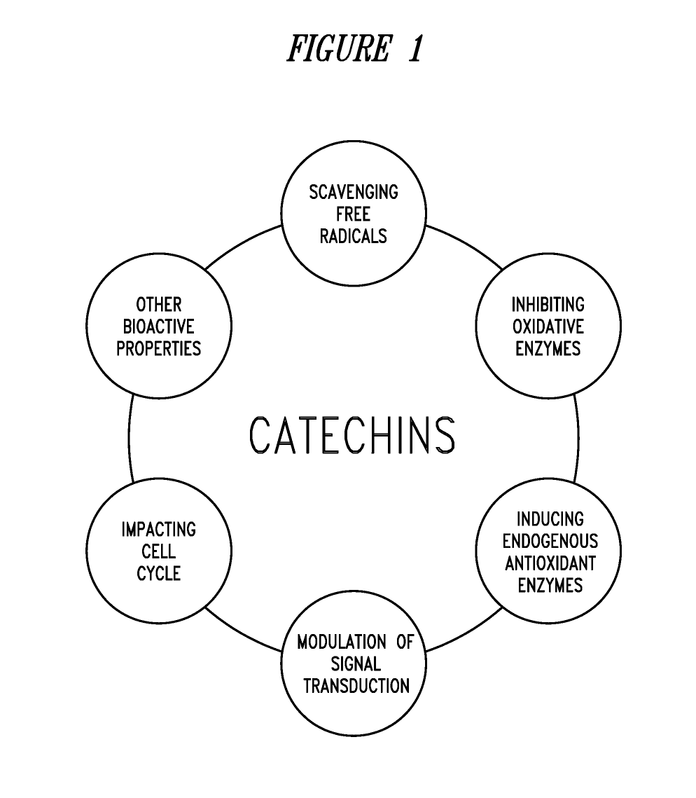

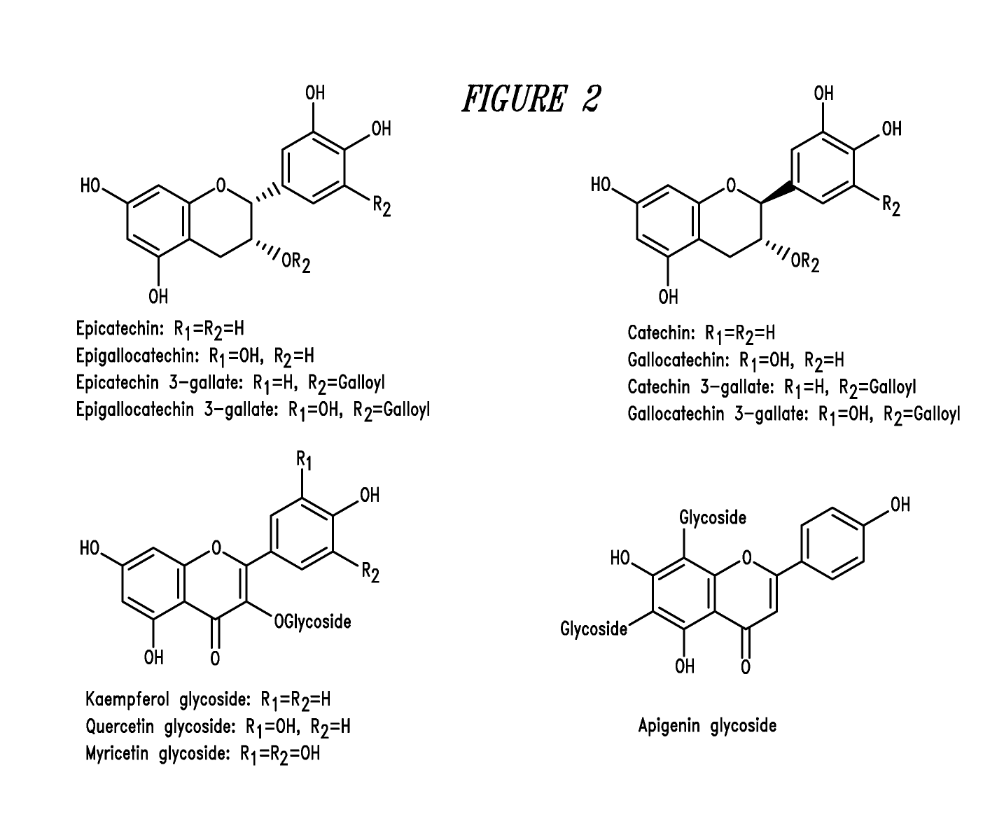

### DETAILED DESCRIPTION

The present disclosure provides green tea plant extracts having improved characteristics, including at least one of bioavailability, therapeutic efficacy, reduced amount needed for biological activity, long-term stability, and increased amount in circulation after ingestion or non-oral administration, as well as for topical use such as dermal application. The methods and products of the invention are described with reference to the use of green tea catechins in human disease prevention and treatment.

The compositions disclosed herein also meet a long-felt need for green tea polyphenols having increased bioavailability in mammals, including humans, for nutritional and therapeutic purposes.

The plasma levels of catechins in humans following oral ingestion were substantially and statistically significantly enhanced as a result of the disclosed Phytofare® green tea extraction process when compared to that observed for a commercial green tea extract. The catechin found at the highest concentration for Phytofare® product was epigallocatechin gallate (EGCG).

The plasma levels observed after administration of the Phytofare® product did not return to zero within 12 hours of administration. The results disclosed in Example 44 show that the circulating half-life of the catechins prepared according to the Phytofare® is much longer and that a baseline level for catechins are maintained when using the disclosed dosing intervals.

Catechins.

The term “catechin” is used herein according to the art-accepted definition as a major component of the tea leaf, constituting between 20-30% of the dry leaf. Catechins are water-soluble and colorless flavenoids derived from the shikimic and acetate-malonate biosynthetic pathways. In more common terminology, catechins are also known as immature tannins. EGCG is the predominant catechin from green tea leaves, along with its stereoisomer GCG.

The structure of the various catechins is relevant to bioavailability and to dietary and medical use. Preliminary studies have shown that cis and trans catechin configurations have different lipid solubility and oxidative potential, as discussed further, below. Regarding the gallic acid esters of some catechin molecules, Uekusa, Y. et al., in a review of the catechin literature (J. Agric. Food Chem. 55:9986-92, 2007), reported that ECG and EGCG showed higher activities in a variety of physiological tests than EC and EGC, which lack the gallic moiety. The authors suggested that these differences may be due in part to the molecules' respective affinities for lipid membranes. In addition, Uekusa reported that cis-catechins may have higher affinity for lipid bilayers than the corresponding trans-catechins. These differences in turn can affect the molecule's bioavailability when used therapeutically and/or nutritionally.

Bioavailability.

“Bioavailability” is defined as a measurement of the amount of a compound absorbed into the bloodstream. The Physician's Desk Reference (PDR) states that for nutrients supplied as pills, tablets, or soft gels, the maximum absorption is between ten and thirty percent. As a result, the majority of a therapeutic nutrient passes through the body unused. Herein the term “nutrient” includes green tea phytonutrients, such as catechins.

Barriers to better absorption include the stomach, which can alter or destroy the nutrient by action of acid; the intestine, which itself may utilize most of the nutrient; and the liver, which can recognize the nutrient as foreign and tag it for elimination. In the intestine, for example, colonic microflora can convert EGC and EGCG to valerolactone forms, which do not possess the biological activity of the original catechin molecules. Other pathways, such as glucuronidase and glucosidase, process ECG and EGCG for elimination.

Currently available high purity green tea EGCG extracts have poor bioavailability of between 1-10%. The green tea plant extracts disclosed herein have significantly increased bioavailability of at least 50%, and preferably between 60-80%. This value far exceeds that of the extracts or preparations used in the prior studies of green tea's health effects discussed below.

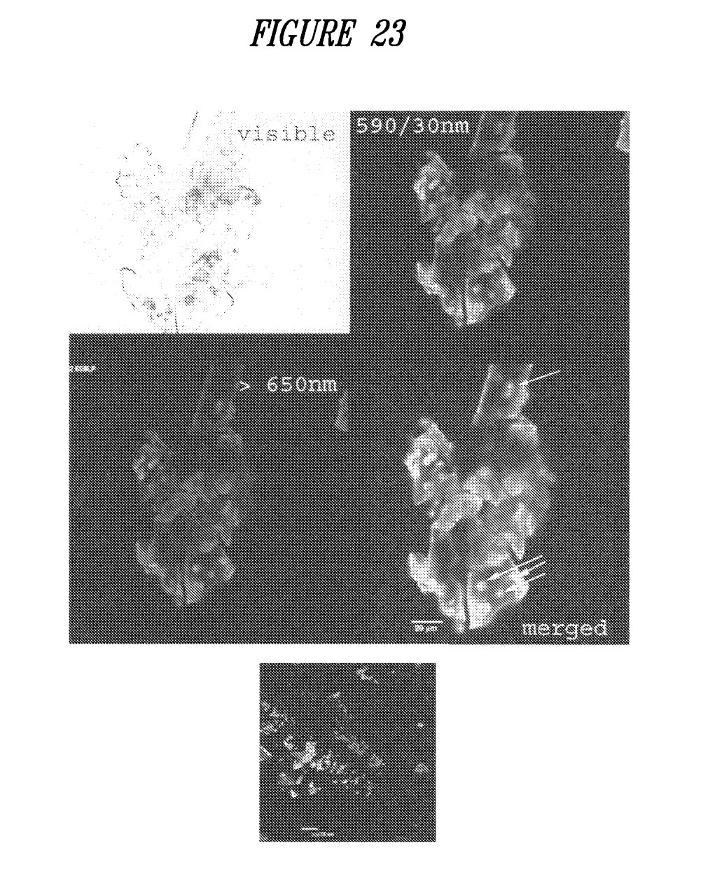

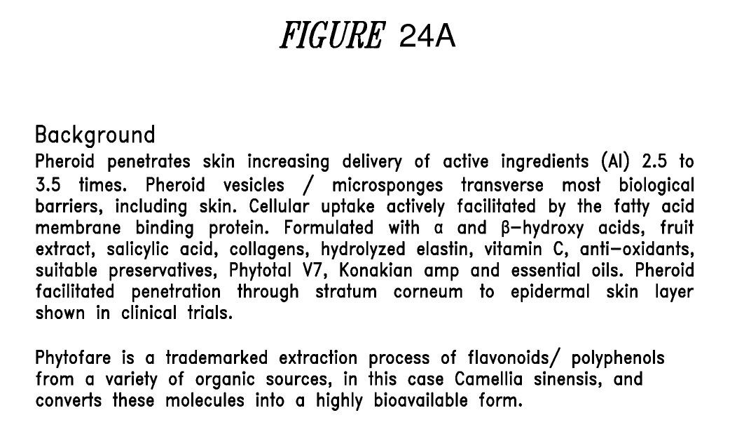

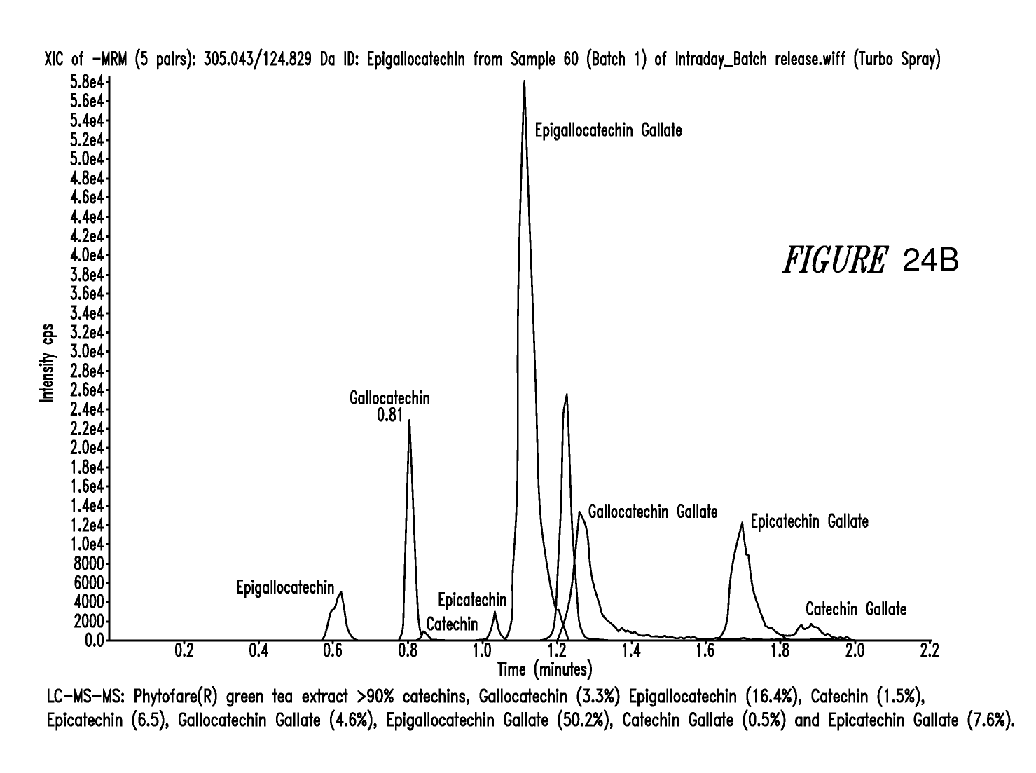

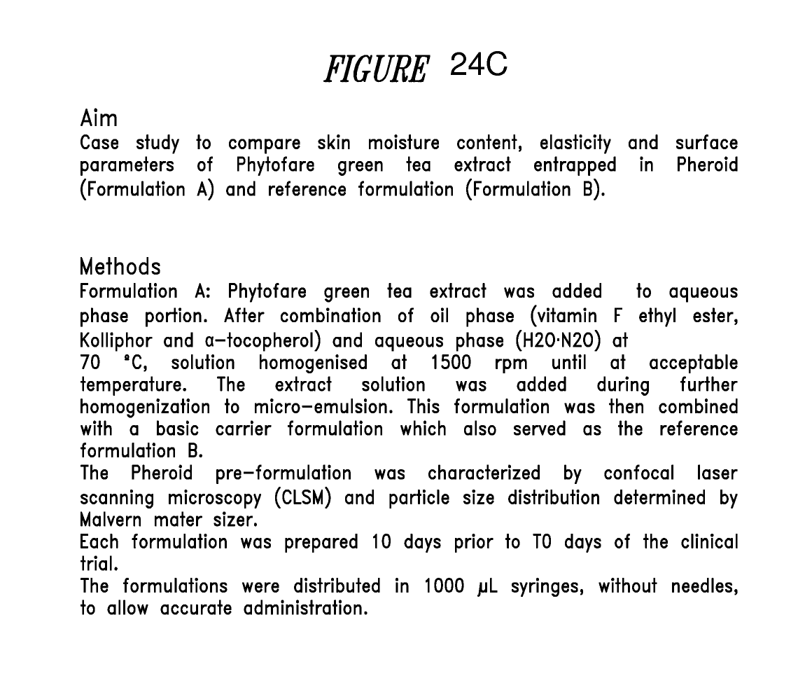

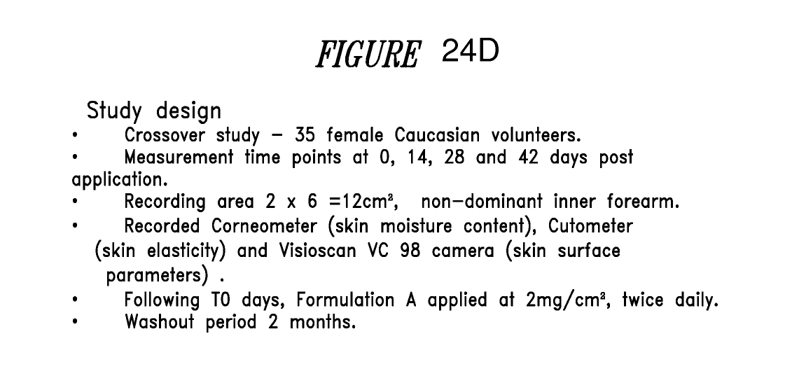

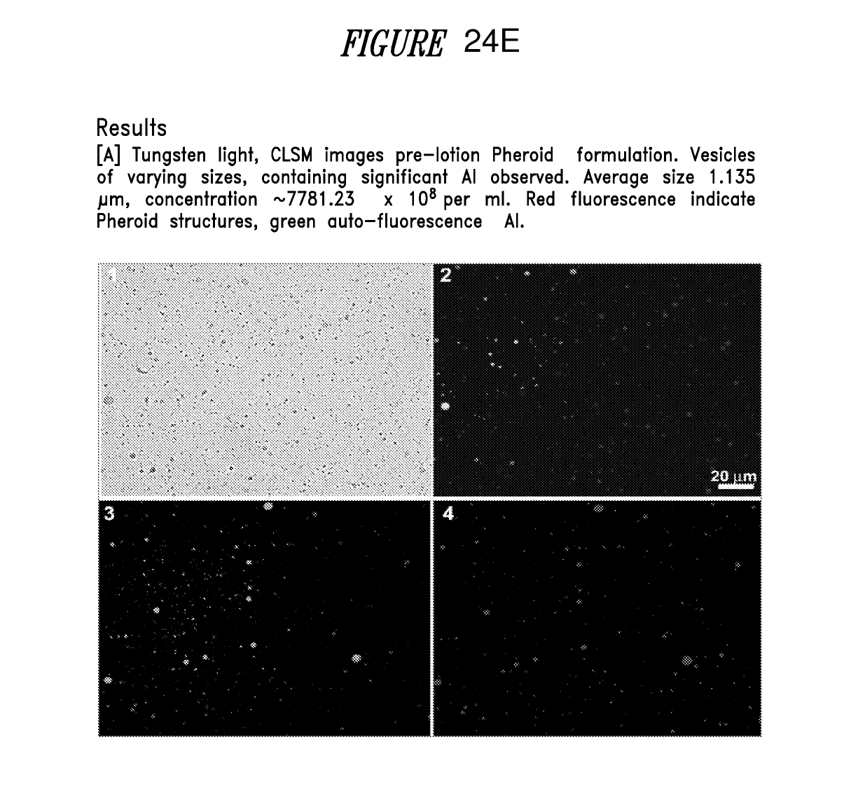

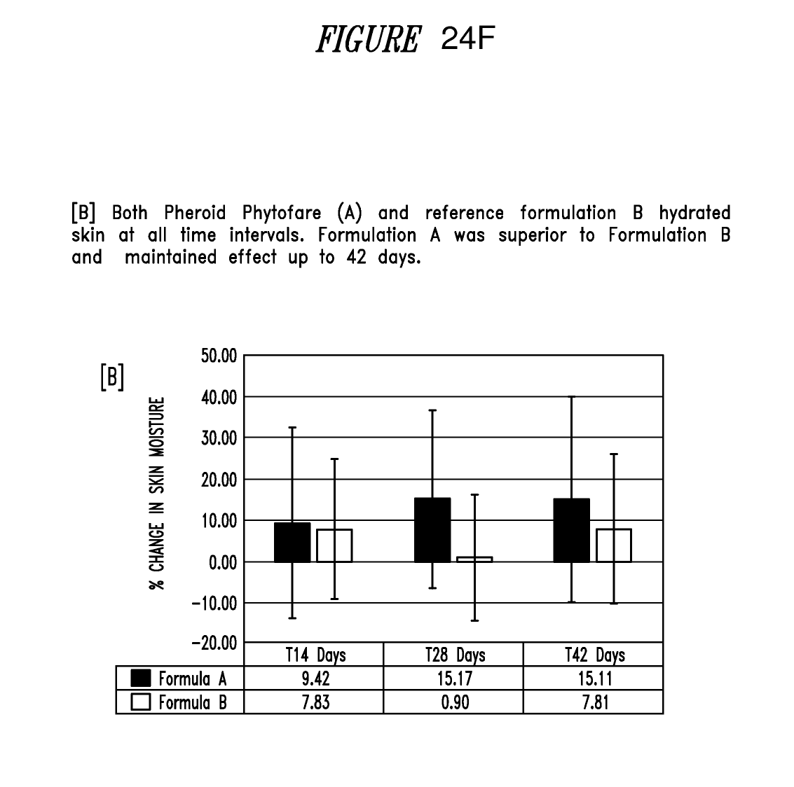

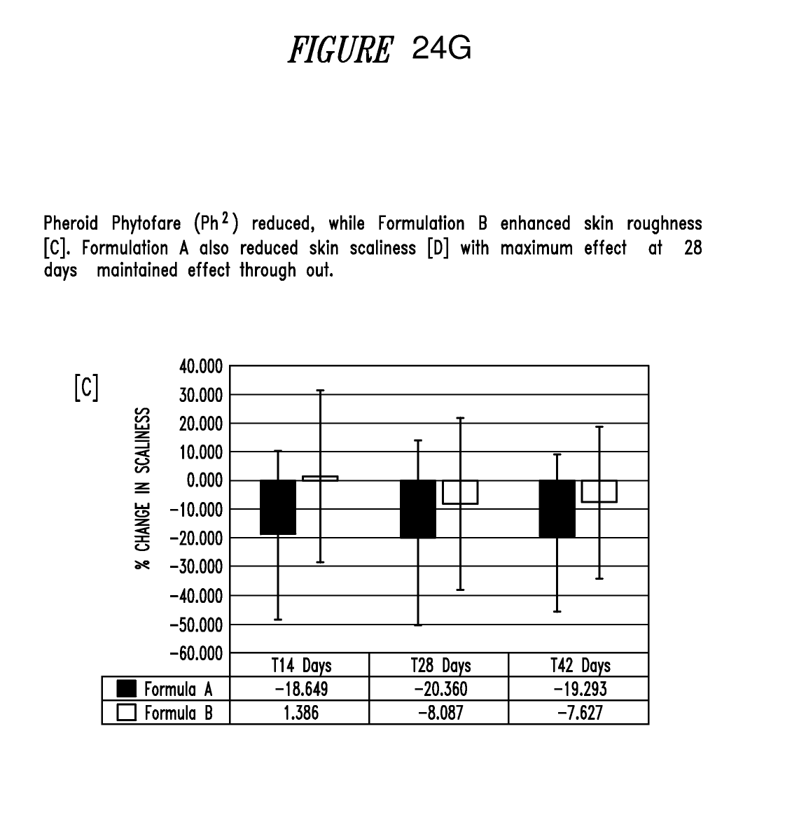

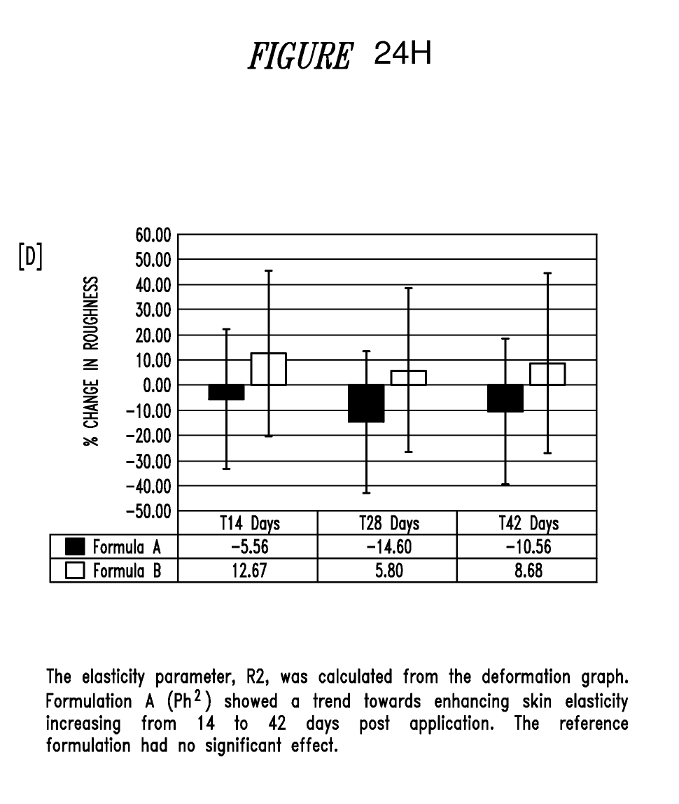

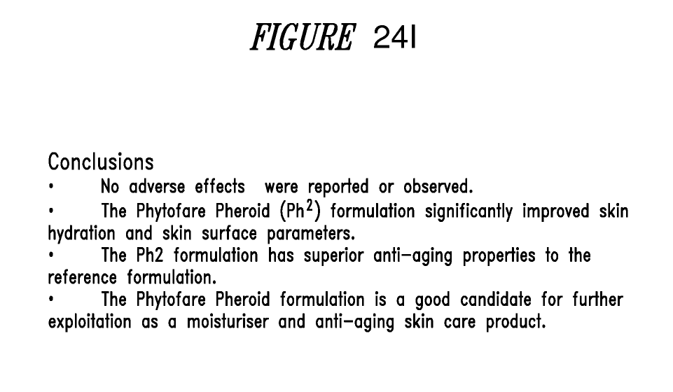

Without being bound by a specific mechanism or mechanisms accounting for the increased bioavailability, in one non-limiting method described herein, the increased bioavailability is accomplished by processing green tea plant material under conditions described in the Examples. The processing results in amorphous crystalline forms as depicted in FIG. 23. The approximate size can include ranges such as from about 30 nm to 900 nm or greater, and the compositions consist of not less than 1% crystals by weight of total weight of composition.

Therefore the compositions of the disclosure can contain in some embodiments not less than 1% amorphous crystals by weight of total weight of composition. The compositions can contain more than 1% amorphous crystals, such as 2%, 3%, 5% or 10% or more by weight of total weight of composition. However the compositions can contain less than 1% amorphous crystals, with the lower limit being determined by the detection method, in which the presence of any amorphous crystals represents a novel and inventive development over the art.

One non-limiting method of this processing is provided in Example 1 entitled “Processing green tea plant material.” In brief, the device operates as follows. Input material, in this case a green tea leaf preparation, is inserted through the input opening into a reservoir tank. A large, slowly moving paddle rotates within the tank to move material from the outer sides towards the center. A shearing processor in the middle of the tank pulls material in from the bottom, shears it, and expels the sheared material out the side, back into the tank. This system accomplishes the shearing and facilitates homogeneous processing. A green tea extract prepared by this method is referred to in some Examples herein as Phytofare®.

Provided herein are commercially scalable compositions of green tea extracts that meet the requirements of bioavailability, stability, and purity. The final products contain a mixture of green tea polyphenols, specifically including catechins C, CG, GC, GCG, EC, ECG, EGC, and EGCG. In one embodiment, the product has been processed to remove caffeine, as described in Example 2.

Bioaccessibility.

“Bioaccessibility” is a measure of a mammal's ability to digest and process green tea catechin components, and can be assayed in vitro using gastric phase and intestinal phase enzymes such as those described by Fleshman, M. K. et al. (Agric. Food Chem. 59:4448-4454, 2011). Fleshman also teaches that bioavailability, in terms of uptake by cells of the digestive system, can be assayed using Caco-2 (HTB-39) cells which mimic mature enterocytes.

Caco-2 cells in combination with HPLC can be used to measure accumulation of catechins by cells, as an indication of intestinal absorption (Takaishi, N. et al., Biosci. Biotechnol. Biochem. 76:2124-2128, 2012). Another method of measuring bioavailability is discussed above with reference to Boileau's work on micellar incorporation of lycopene cis and trans forms; these assays can be applied to the catechin preparations and green tea extracts of the present disclosure. (J. Nutr. 129:1176-1181, 1999.)

Other assays are available to measure bioavailability and bioaccessibility of green tea catechins. Ishii, T. et al. (Mol. Nutr. Food Res. 54:816-822, 2010) describe the use of a human serum albumin (HSA) column to separate tea catechins. The catechin binding to HSA depends on structure, including the cis or trans isomerization, and after elution the catchins are analyzed by high pressure liquid chromatography (HPLC). The HSA binding assay is indicative of bioaccessibility as a measure of transportation in the circulation after ingestion.

Carrier Systems

The green tea extracts disclosed herein, and/or one or more catechins isolated therefrom, can be incorporated in a carrier system referred to as Pheroid™ for in vivo administration. In one non-limiting example described in more detail in Example 9, a Pheroid™-EGCG formulation was compared to EGCG alone. The Pheroid™-EGCG formulation resulted in increased plasma concentration of EGCG over the initial three hours after administration. The Pheroid™ technology is described in for example U.S. Patent Publication No. 20120302442, Nov. 29, 2012, for “Plant Support Formulation, Vehicle for the Delivery and Translocation of Phytologically Beneficial Substances and Compositions Containing Same,” which is incorporated by reference herein (including continuation filed Jul. 10, 2014, Patent Publication No. 20140194288).

The green tea catechols having increased bioavailability, as described herein, can be provided in their natural mixture, such as including all the catechins C, CG, GC, GCG, EC, ECG, EGC, and EGCG. For some uses, one or more of the catechins may be isolated and provided separately, for example for use to supplement green tea extract preparations and/or to supplement other non-catechin therapeutic compositions.

Thus, a pharmaceutical or nutritional preparation disclosed herein can contain C, CG, GC, GCG, EC, ECG, EGC, or EGCG alone; it can contain any two of these catechins, any three of these catechins, any four of these catechins, any five of these catechins, any six of these catechins, or any seven of these catechins.

Compositions with one or more of the individual catechins meet the needs for both research and therapeutic use of the compositions. As reported by Shimizu, M. et al., Clin. Cancer Res. 11:2735-2746 (2005), epigallocatechin gallate (EGCG) in combination with epicatechin (EC) caused synergistic inhibition of growth and induction of apoptosis in HT29 cells, a human colorectal cancer cell line. The authors suggested that the synergistic result may be due to enhanced cellular uptake of EGCG by EC, citing Suganuma M. et al., Cancer Res 59:44-7 (1999). These and other studies suggest a need in the art for more bioavailable green tea catechins, individually and as mixtures, to elucidate the therapeutic mechanisms and to provide the synergistic effects obtained using the whole tea extract.

Medical Uses of Green Tea Polyphenols.

There are published suggestions that green tea consumption can alleviate a variety of pathological conditions and may prove effective against infectious agents including parasites, viruses, and bacteria. However, without consistent, repeatable dosing with bioavailable material, the studies to date have not led to development of any non-experimental pharmaceutical products, or to significant investment in developing such products.

The present disclosure helps to remedy this situation, and the disclosed polyphenol compositions, including Phytofare®, are suitable for many research and clinical uses, not limited to those discussed herein. Although the publications discussed below may suggest mechanisms for the effects, those mechanisms are not intended to be limiting because that data was developed without use of standardized dosing of bioavailable polyphenols as disclosed herein.

Parasitic Disease.

Malaria is just one of the many human diseases caused by parasites. Malaria has been particularly difficult to prevent and treat, due in part to the complex life cycle of the malaria parasite and its ability to avoid the immune system once it has established an infection in a human host. Many malaria isolates have been identified in humans, with P. falciparum, P. vivax, P. ovale, and P. malariae being the most common.

There is evidence that green tea catechins have specific anti-malarial properties. Gallate catechins are the best studied to date. The present disclosure provides evidence of clinical efficacy against Plasmodium falciparum. This is described in detail below in Examples 7 and 8.

Much effort to design vaccine interventions against malaria has focused on the blood stage of the parasite, which is responsible for the symptoms of the disease. However, prevention of infection by interfering with Plasmodium function earlier in the process continues to be a goal. The green tea extract compositions disclosed herein help to meet that goal, either alone or in combination with one or more existing malaria treatments, such as artemisin.

Plasmodium malariae has distinct developmental cycles in the Anopheles mosquito and in the human host. The mosquito serves as the definitive host and the human host is the intermediate. When the Anopheles mosquito takes a blood meal from an infected individual, gametocytes are ingested from the infected person and within the erythrocyte cycle up to eight mobile microgametes are formed.

During this erythrocyte cycle, malaria gliding parasites invade the body by relying on the mammalian facilitative glucose transporters and the 72 hour cycle begins. Glucose is the primary source of energy and a key substrate for most cells. For malaria, blood forms of parasites rely almost entirely on glycolysis for energy production and as there are no energy stores, the parasites are dependent on the constant uptake of glucose.

Bioavailable green tea gallate catechin extracts of the present disclosure are suitable for inhibiting the hexose uptake processes in infected erythrocytes. Gallate catechins are also able to inhibit motility and cause cytotoxicity through the formation of several hydrogen structures and ionic bonds with proteins, thereby modulating their three-dimensional structures. Gallate catechins bind to adhesion molecules on the parasite surface and this impairs gliding, leading to an inactivation of the surface proteins and rendering the parasites immotile.

The need for bioavailable, standardized green tea catechin compositions for malaria studies is noted in the literature. For example, Slavic, K. et al. (Mol. Biochem. Parasitol. 168:113-116, 2009) studied the effects of green tea catechins on hexose transporters and concluded that, “Our results provide new data on the inhibitory action of catechins against sugar transporters but were unable to elucidate the antimalarial mechanism of these agents.”

This and other work performed to date supports the need for green tea-derived bioavailable compositions as malarial treatments through several documented mechanisms. (a) Gallate catechins bind to the intracellular adhesion molecule 1, also known as ICAM-1, on endothelial cells. Plasmodium-infected erythrocytes are unable to adhere to endothelium if ICAM-1 is blocked. (b) Gallate catechins inhibit P. falciparum growth in vitro, and can act in concert with the existing anti-malarial drug artemisin, thus potentiating its action. (c) Gallate catechins interfere with fatty acid biosynthesis in P. falciparum, by inhibiting an enoyl-acyl carrier protein reductase of the parasite. (d) Gallate catechins inhibit hexose uptake in infected erythrocytes, thus depriving the parasites of their primary energy source. (e) Gallate catechins can inhibit motility of the parasites, by binding to adhesion molecules on the parasite surface and inhibiting gliding.

Examples 7-9 below provide details of using the compositions of the disclosure to elucidate the mechanisms responsible for the observations regarding green tea catechins and malaria.

Anti-Aging, Skin.

Example 10 discloses the use of compositions comprising green tea extract for topical use on skin. The results described in Example 10 show that the green tea-containing compositions promoted skin hydration, decreased skin roughness, decreased skin scaliness, and increased skin elasticity, indicating the utility as anti-aging skin preparations for human use.

Research has shown that drinking green tea can affect the three major causes of acne: inflammation, insulin resistance and hormones. Current research suggests that acne is a result of inflammation caused by oxidative stress primarily in the gut but also elsewhere in the body, not confined to just the skin. Cytokines produced during inflammation stimulate growth of cells in the skin that block pores and subsequently reduce the removal of toxins, dead cells, and other materials, and this in turn lowers oxygen levels so that P. Acne bacteria are able to thrive and create more inflammation in the skin.

Some studies report use of topical tea and green tea preparations for treatment of these conditions, including Mahmood, T. et al., Bosnian J. of Basic Medical Sciences 10:260-264, 2010 (3% green tea emulsion decreases skin sebum production) and Sharquie, K. E. et al., Saudi Med. J. 29:1757-1761, 2008 (topical therapy using 2% tea lotion). Thus, the green tea preparations as disclosed herein offer an option for alleviating acne and related skin conditions.

HIV.

One approach to preventing the spread of human immunodeficiency virus (HIV) is to inhibit viral integration into an infected cell. The virus produces an enzyme known as integrase, and inhibition of this enzyme has been a goal of prior research. An HIV-1 integrase target drug, Raltegravir, was approved by the FDA in 2007, but viral mutants resistance to the drug are being found in clinical trials (for example, Hu, Z., J. Acquir. Immune Defic. Syndr. 55:148-55, 2010).

Research has demonstrated that four green tea catechins with the galloyl moiety, including CG, EGCG, GCG, and ECG, inhibited HIV-1 integrase (Jiang, F. et al., Clin. Immunol. 137:347-356, 2010). Thus, compositions disclosed herein can provide an alternative and/or a supplement to HIV-1 integrase inhibitors already on the market, which may eventually lose their effectiveness due to virus resistance mutations.

Nance, C. L. et al., J. Allergy and Clin. Immunol., 123:459-465 (2009) reported that EGCG at physiological levels (i.e. achievable by green tea consumption) blocked the binding of HIV-1 gp120 to CD4 on T cells; CD4 is the primary receptor for HIV and therefore an important target for therapy. In other experiments, the physiological concentrations of EGCG used in vitro to inhibit HIV-1 infectivity (measured by p-24 antigen production) was 4.5 μmoles/L to 6 μmoles/L.

Green tea antioxidant catechins (a group of green tea polyphenols), especially EGCG, the strongest antioxidant catechin, have anti-HIV activity “in each step of the HIV life cycle” according to Yamaguchi, K. et al., Antiviral Res. 53:19-34, 2002.

Results of green tea HIV prevention research show that green tea catechins, particularly EGCG, destroy viral particles; block viral attachment to cells; prevent viral entry into cells; slow reproduction of viruses; protect RNA and DNA integrity to reduce mutations; can be effective with drug resistant viruses; and protect against secondary damage from viruses.

HIV can enter T4 cells (anti-viral lymphocytes, or white blood cells from the immune system) at a site called the CD4 molecule on the T4 cell wall. The HIV virus uses its envelope glycoprotein (gp120) to attach to the CD4 site. When attachment is successful, the virus can enter the cell, taking over the genetic material, replicating, using up all the cell's resources, killing the cell, then exiting and repeating the process until the infected individual dies.

Several studies show that EGCG, the primary green tea catechin bonds more strongly to CD4 than the HIV virus gp120, thus blocking gp120 and preventing HIV from entering the T4 cell. Using flow cytrometry, EGCG is seen bonding directly to CD4, inhibiting gp120 bonding and blocking HIV entry into cells (Kawai, K. et al. J. Allergy Clin. Immunol. 112:951-7, 2003). EGCG binds to CD4 with a stronger chemical affinity than gp120, thus blocking gp120-CD4 binding (Hamza, A. et al. J. Phys. Chem. B. 110:2910-7, 2006). Using nuclear magnetic resonance spectroscopy, EGCG showed strong binding to CD4 which reduced gp120 binding.

Green tea slows reverse transcriptase (HIV-1 RT). HIV needs a viral enzyme, reverse transcriptase, to make a DNA copy before it can reproduce. Inhibiting reverse transcriptase reduces the capacity of HIV to reproduce. EGCG from green tea strongly inhibited replication of two strains of HIV as determined by reverse transcriptase inhibition (Fassina, G. et al., AIDS 16:939-41, 2002). EGCG, EGC and ECG and GTE (green tea extract) are all potent inhibitors of HIV-1 RT (Chang, C. W. et al., J. Biomed. Sci. 1:163-166, 1994.). Both EGCG and ECG strongly inhibited reverse transcriptase (Nakane, H. et al., Nucleic Acids Symp. Ser. 21:115-6, 1989; Biochemistry 29:2841-5, 1990). One study found that the weaker green tea catechins did not slow reverse transcriptase, but green tea catechins together with EGCG significantly inhibited reverse transcriptase (Tichopad, A., J. Ethnopharmacol. 99:221-7, 2005).

Green tea has been shown to stimulate production of healthy lymphocytes up to 300%. Green tea has also been shown to stimulate production of immune system killer cells up to 400%. Green tea protects against many secondary intestinal infections which can lead to “wasting.” Green tea may protect against AIDS-related dementia. In the majority of people with HIV AIDS, the central nervous system is infected with HIV. The most severe cases develop into dementia, mediated by the activation of pro-inflammatory cytokines. These cytokines (IFN-γ) make HIV-1 more toxic to nerve cells and enhance the actions of HIV gp120.

AIDS prevention. If green tea catechins eventually prove to be an important part of preventing HIV infections, one consideration is supply. Currently, enough tea is grown and transported to allow ⅔ of the world's population to drink tea daily. While most of the tea is processed into black tea, it is actually easier to process tea leaves into green tea. The cost of drinking ten cups of green tea every day or taking green tea extract supplements in the United States averages about $15 a month. Greater bioavailability of green tea extracts of the disclosure can lower the amount required for prevention.

Both HIV and the Ebola virus bud from infected cells by use of the protein Tsg101. In view of the shared mechanism of action in HIV and Ebola, other shared mechanisms may be present, leading to the potential use of the green tea compositions of the disclosure. Von Schwedler, U. K. et al., “The protein network of HIV budding,” Cell 114:701-13. (2003). HIV and Ebola also use the same mechanism of action for entry into cells. The mechanism of these viruses is through the protein gp120 that binds the virus to the white bloods cells' CD4 on the T cell.

Studies have been undertaken with Polyphenol E (green tea catechin complex produced from dry leaf), an FDA approved botanical drug 2006 for cancer prevention. Clinical studies at the Baylor School of Medicine have shown the ability of the catechins to prevent binding of the virus protein gp120 to the white blood cells CD4, but requiring excessive dosage and with poor bioavailability of 1-2%. The green tea extracts disclosed herein with significantly increased bioavailability allow testing for their ability to block binding of HIV to CD4.

Barrientos L. G. et al., Antiviral Res. 58:47-56 (2003) studied the potential use of HIV-inactivating protein cyanovirin-N (CV-N) as a therapy for Ebola. CV-N is known for its ability to inhibit the infectivity of a broad spectrum of HIV strains at the level of viral entry. The mechanism of action involves CV-N binding to N-linked high-mannose oligosaccharides on the viral glycoprotein gp120.

Because the Ebola envelope contains oligosaccharide constituents similar to HIV gp120, Ebola may be susceptible to inhibition by CV-N. Initial results reported by Barrientos et al. showed that CV-N had both in vitro and in vivo antiviral activity against the Zaire strain of the Ebola virus (Ebo-Z). Specifically, addition of CV-N to the cell culture medium at the time of Ebo-Z infection inhibited the development of viral cytopathic effects (CPEs). CV-N also delayed the death of Ebo-Z-infected mice. This was found both when given as a series of daily subcutaneous injections, and when the virus was incubated ex vivo together with CV-N before inoculation into the mice.

In addition, results were found similar to earlier experiments with HIV gp120: CV-N binding with affinity to the Ebola surface envelope glycoprotein, GP(1,2). Barrientos et al. performed competition experiments with free oligosaccharides, and the results were consistent with the conclusion that carbohydrate-mediated CV-N/GP(1,2) interactions involve oligosaccharides residing on the Ebola viral envelope. The studies by Barrientos et al. therefore implicate carbohydrate moieties on viral surface proteins as common viral molecular targets for CV-N protein, and point to the value of testing green tea extracts of the present disclosure for use in inhibiting Ebola binding to white blood cells.

Brain Function.

In a 2014 study, Schmidt, A. et al. reported that “green tea extract increased the working memory induced modulation of connectivity from the right superior parietal lobule to the middle frontal gyrus. Notably, the magnitude of green tea induced increase in parieto-frontal connectivity positively correlated with improvement in task performance.” Study participants consumed a drink containing 27.5 green tea extract while undergoing functional magnetic resonance imaging (fMRI), and controls received the drink without the green tea extract. The authors concluded that the green tea provided a beneficial effect on working memory processing at the neural system level, in terms of changes in short-term plasticity of parieto-frontal brain connections. (Psychopharmacology 231:3879-3888, 2014)

These results are consistent with other studies on healthy individuals. Kuriyama, S. et al. reported that higher consumption of tea was associated with a lower prevalence of cognitive impairments in older adults. (Am. J. Clin. Nutr. 83:355-361, 2006). Similar results were shown in a later studies by Ng, T. P. et al. (Am. J. Clin. Nutr. 88:224-231, 2008) and Feng, L. et al. (J. Nutr. Health, and Aging 14:433-438, 2010). These three studies together involved thousands of individuals, and found tea consumption to be positively associated with improved cognitive performances in separate groups of adults over 55.

In addition to being beneficial to the cognitive performance of healthy individuals, tea, including green tea, shows promise for alleviating dementia and Alzheimer's Disease. For example, EGCG was reported to be beneficial for Alzheimer's indicia in Alzheimer's transgenic mice. Effects included modulating amyloid precursor protein cleavage and reducing cerebral amyloidosis (Rezai-Zadeh, K. et al., J. Neurosci. 25:8807-8814, 2005), and reducing β-amlyoid mediated cognitive impairment and modulating tau pathology (Rezai-Zadeh, K. et al., Brain Res. 1214:177-187, 2008).

On the basis of these and other studies on green tea and brain function, the green tea extracts disclosed herein are suitable for similar uses, particularly in view of the disclosed increased bioavailability and suitability for standardization.

Cancer.

An FDA-approved pharmaceutical-grade green tea product in cancer clinical trials as of 2012 is Polyphenon E (also known as Poly E). Poly E is composed of about 60% EGCG, 12% EGC, 7% EC, 2% GCG, and 1% ECG (Shimizu, M. et al., Clin. Cancer Res. 11:2735-2746, 2005). EGCG is the most abundant catechin in the mixture.

Bode, A. et al., Cancer Prev. Res. 2:514-517 (2009) reviewed the Polyphenon E and green tea studies through 2009, citing 56 references, and reported on numerous in vivo and in vitro studies. One significant finding relates to use of a mixture of catechins versus individual catechins.

As reported by Bode, several studies compared Poly E to EGCG. Bode et al. concluded that, “The available clinical evidence suggests that Poly E is more bioavailable than is EGCG alone, which may explain the differences in efficacy between the two agents in different models.” Citing Shimizu, M et al., Clin. Cancer Res. 11:2735-2746 (2005), who studied the effects of catechins on human colon cancer cells, Bode stated that, “as little as 1 μg/mL of epicatechin combined with EGCG (10 μg/mL) had synergistic inhibitory effects on cell growth . . . suggesting an interdependency for optimal activity. Accumulating evidence from animal models strongly suggests that EGCG and perhaps other catechins are not as effective in vivo on their own as they are combined.”

Bode's conclusion is consistent with the approach taken in the present disclosure for extracting and processing a natural mixture of green tea catechins to improve bioavailability. According to Bode, “EGCG or other polyphenol chemicals may require their complex, natural combination forms to be active anticancer agents because they depend on interactions with other whole-food components for efficacy . . . .”

In 2012, Fujiki, H. et al., J. Cancer Res. Oncol. 138:1259-1270, reported that drinking 10 Japanese-size cups of green tea per day delayed the cancer onset in women, and drinking 10 cups of green tea per day plus green tea tablets had a preventive effect on recurrence of colorectal adenomas. These results, based on use of whole green tea phytonutrients, supports Bode's conclusions. Currently, however, Poly E is only available for clinical trials

The present disclosure is fully consistent with the current state of the art's need for potent and stable catechin-containing compositions that allow more precise and repeatable testing in standardized assays for anti-cancer activity. These assays include those used by Dou Q. P. et al., Inflammopharmacology 16:208-212, 2008, including cancer cell lines such as breast cancer MDA-MB-231 cells, and those use in studies cited by Bode, discussed above.

Other suitable assays and cell lines include MCF-7 and MDA-MB-231 cell lines, as used by Kim, J. et al., Food Funct. 4:258-265, 2013; MDA-MB-235 cell line, Guthrie, U.S. Pat. No. 6,251,400, 2001; mouse metastatic mammary cell line 4T1, used by Morro to test tea catechins (U.S. Pat. No. 6,410,061); and human colon cancer cell line HCT116, used by Netke to test EGCG alone and in combination with other compounds (U.S. Pat. No. 6,939,860).

For evaluating green tea compositions for cancer treatment and prevention in vivo, Morré (U.S. Pat. No. 6,410,061) used 4T1 cells to grow tumors in mice, and then evaluated the effect of EGCG on tumor size and vascular endothelial cell growth. Blood vessel cell growth in vitro, as an indicator of anti-angiogenic effects in vivo, was tested by Pratheeshkumar, P. et al., Eur. J. Pharmacol. 668:450-458, 2011, and these assays can be applied to testing green tea extracts and catechins disclosed herein. Sakamoto, Y. et al., Biosci. Biotech. Biochem. 77:1799-1803, 2013, describes the use of EGCG to reduce tumor cell growth when fed to mice.

These publications are incorporated by reference herein for their teachings and assays.

Inflammation.

Inflammation plays a major role in many chronic medical conditions, including blood pressure, blood sugar levels, and insulin resistance. One study showed that green tea extract consumption by obese individuals was associated with reduced blood pressure, improvements in insulin levels and blood sugar levels, and reduction of the inflammatory marker C-reactive protein (CRP). (Bogdanski, P. et al., Nut. Res. 36:421-427, 2012.) That green tea preparation reportedly contained 379 milligrams of polyphenols, but as discussed previously, the bioavailability is low, so a lower amount of polyphenols would have been presented to the tissues.

The present disclosure provides an improved green tea polyphenol preparation for use in clinical trials to further elucidate the mechanisms by which the prior art green tea polyphenol extracts alleviated inflammation-associated conditions. The disclosed preparations are suitable for targeting specific medical conditions with appropriate dosages.

The disclosed green tea compositions are assayed for anti-inflammatory effects using, for example, Mono Mac 6 cells as a model of monocytes/macrophages in inflammation (Koganov, U.S. Pat. No. 8,318,200, 2012), and assays disclosed in Kong, K.-W. et al., Molecules 15:959-987, 2010.

Diabetes.

Although green tea was suggested as a potential treatment for diabetes in the early-mid 1900's, the lack of an effective pharmaceutical composition from green tea has hampered clinical studies. A preliminary report indicates the need for the compositions of this disclosure, by suggesting that a green tea extract with a high content of EGCG can make a positive difference in an animal model of diabetes. Db/db mice were used in the study. As reported by Ortsater, H. et al., Nutrition and Metabolism 9:11 (2012), EGCG supplementation improved glucose tolerance and increased glucose-stimulated insulin secretion. Other effects included a reduction in islet endoplasmic reticulum stress markers, which the authors suggested might be linked to the anti-oxidative capacity of EGCG.

That diabetes study was performed with a green tea extract containing a variety of ingredients in addition to the high content of EGCG. The promising results further support the need for standardized compositions containing green tea catechins in bioavailable form, for controlled clinical studies on the use of green tea for treating diabetes.

Assays relevant to the role of green tea compositions in obesity and diabetes are disclosed, for example, by Eidenberger, U.S. Pat. No. 7,862,840, 2011, for measuring inhibition of lipase. Lipase inhibition can reduce lipolysis in vivo and reduce the absorption of fat, for treating obesity. Forester, S. C. et al., Mol. Nutr. Food Res. 56:1647-1654, 2012, reported that in mice fed starch, EGCG reduced the increase in blood sugar levels, and that EGCG and EGC inhibited α-amylase in vitro, as a measure of starch metabolism. Specifically, Forester et al. examined the inhibitory effect of EGCG and (−)-epigallocatechin (EGC) on α-amylase activity in a cell-free system, using a commercially available assay (chromogenic Red-starch method from Megazyme, Wicklow, Ireland).

According to Forester, both EGCG and EGC inhibited α-amylase activity with EGCG being the more potent inhibitor. At a concentration of 20 μM, EGCG inhibited α-amylase by 34%, whereas EGC caused only 13% inhibition at the same concentration. Kinetic analysis showed that 50 μM EGCG reduced the Vmax of α-amylase from 323.3 to 206.1 product/min/mg protein (p<0.01), but did not significantly affect the Km (p=0.1) in the presence of increasing concentrations of Red-starch substrate. Forester's results suggest that EGCG inhibits α-amylase in a noncompetitive fashion with regard to substrate concentration. These assays are suitable for documenting the utility of the disclosed green tea extracts for treating and/or preventing obesity and diabetes, and an α-amylase assay is described in Example 15.

Before controlled studies can be done on these and other uses of green tea catechin in medicine and nutrition, the problems of low oral bioavailability, standardized dosing, and stability had to be resolved. The compositions of the present disclosure are suitable for meeting this challenge.

Example 1 describes the use of a hydrodynamic process applied to the plant material. This process provides one non-limiting method of achieving the improved bioavailability of plant phytonutrients, particularly catechins according to the disclosure. The process was used to prepare tomato extracts that were then evaluated by the U.S. Department of Agriculture, and the investigators published their findings (Ishida, B. et al., Food Chemistry 132:1156-1160, 2012). The authors concluded that this method of treating vegetable matter changed the stereoisomeric profile of lycopene to one that is more bioavailable.

Green tea catechins can also be prepared synthetically. EGCG synthesis is described in Li, L. et al., Org. Lett. 3:739-741 (2001), and ECG synthesis is described in Wan, T. H., Tetrahedron 60:8207-8211 (2004). Analog synthesis is described in Landis-Piwowar, K. R. et al., Int. J. Mol. Med. 15:735-742 (2005); Smith, D. M. et al., Mol. Med. 8:382-392 (2002); and Wan, S. B. et al., Bioorg. Med. Chem. 13:2177-2185 (2005). Chemical synthesis of EGCG is also described in Nagle, D. G. et al., Phytochemistry 67:1849-1855 (2006).

In Vitro Systems and Animal Models.

It is customary in biomedical research to use in vitro cell culture systems and animal models to investigate dosing, safety, mechanism of action, and other parameters of a compound prior to human use. In addition to the animal models discussed above for specific conditions, other animal models are suitable for use with the compositions disclosed herein.

Lambert, J. D. et al., Mol. Pharm. 4:819-825 (2007) studied biotransformation and biological activity of green tea polyphenols. Biochemical transformations in the mammalian tissues can affect the ultimate bioavailability of green tea polyphenols, including methylation, glucuronidation, sulfation, and ring-fission metabolism.

Mammalian species differ in the catalytic rate of these pathways, and in vitro work indicates that mice are more similar to humans, compared to rats, in the biotransformation of tea catechins. Thus, Lambert et al. suggest that mice are an appropriate animal model for studying the health effects of green tea compounds from the perspective of biotransformation.

Mice are also a suitable animal model for studying the anti-malarial activity of the green tea catechin compositions disclosed herein. Human Plasmodium strains generally do not infect mice, so mouse studies can be performed with the strain P. berghei.

Surrogate models of malaria and mammalian sugar transport have been developed using Xenopus laevis oocytes, and preliminary work suggests that catechins display inhibitory action against these sugar transporters. The principal hexose transporter expressed in the parasite plasma membrane, PfHT, is a sodium-independent, saturable, facilitative hexose transporter. Expression of PfHT in Xenopus laevis oocytes enabled its detailed functional characterization. (Slavic K, et al. Malaria Journal 10:165, 2011.) Other transporters suitable for potential inhibition by catechin compositions of the disclosure include GLUT1 and GLUTS transporters.

For studying cancer, the effect of green tea catechins of the disclosure can be initially tested using a suitable cell line as known in the art. For example, Bode (Bode, A. et al., Cancer Prev. Res. 2:514-517, 2009) discusses a number of colon cancer cell lines that exhibited inhibition of cell growth upon exposure to EGCG or Polyphenon E: Caco 2, HCT116, HT29, SW480, and SW837. These and other suitable cancer cell lines and their corresponding normal cells will be familiar to one of skill in the art. Pharmaceutical compositions. The Examples herein demonstrate that green tea catechin bioavailability was increased. These catechins can be prepared in a form suitable for intended use, whether laboratory research, animal administration, or human administration. With the knowledge provided herein, one of skill can calculate suitable dosages for experimental use in vitro and with non-human mammals.

One intended use of the disclosed catechin compositions is as an adjunct treatment with one or more non-catechin treatments, such as a cancer chemotherapeutic drug or procedure, a malaria treatment, or other appropriate treatment for the selected condition.

Although the catechins can be incorporated as part of the same pharmaceutical composition, to preserve the stability and function they are preferably administered physically separate from any other treatments, either concurrently or in accordance with another treatment schedule. Thus, the compounds or compositions of the disclosure can be used as an adjunct or complement to other therapies.

The pharmaceutical composition comprising a green tea extract composition of the disclosure can be formulated in a variety of forms, e.g., as a liquid, gel, lyophilized, or as a powder or compressed solid. The preferred form will depend upon the particular indication being treated and will be apparent to one of ordinary skill in the art. For oral administration, green tea extract particles can be formulated dry in capsules, with no other ingredients.

An exemplary capsule form can contain the following ingredients per capsule: 150 mg of green tea extract and 10 mg Vitamin C (as ascorbityl palmitate); the capsule can be formulated from hypo-allergenic plant fiber (cellulose) and water. Another exemplary capsule form can contain 100 mg of green tea extract and 3 mg Vitamin C (as ascorbityl palmitate); the capsule can be formulated from hypo-allergenic plant fiber (cellulose) and water.

The bioactive catechin ingredients can also be entrapped in microcapsules prepared, for example, by coascervation techniques or by interfacial polymerization, for example hydroxymethylcellulose, gelatin or poly-(methylmethacylate) microcapsules, in colloidal drug delivery systems (for example liposomes, albumin microspheres, microemulsions, nano-particles and nanocapsules) or in macroemulsions. Such techniques are disclosed in Remington's Pharmaceutical Sciences.

Suitable examples of sustained-release preparations of catechins include semi-permeable matrices of solid hydrophobic polymers containing the compound or composition, the matrices having a suitable form such as a film or microcapsules. Examples of sustained-release matrices include polyesters, hydrogels (for example, poly(2-hydroxyethyl-methacrylate) or poly(vinylalcohol)), polylactides, copolymers of L-glutamic acid and ethyl-L-glutamate, non-degradable ethylene-vinyl acetate, degradable lactic acid-glycolic acid copolymers such as the PROLEASE® (Alkermes, Inc., Waltham, Mass.) technology or LUPRON DEPOT® (injectable microspheres composed of lactic acid-glycolic acid copolymer and leuprolide acetate; Abbott Endocrine, Inc., Abbott Park, Ill.), and poly-D-(−)-3-hydroxybutyric acid. While polymers such as ethylene-vinyl acetate and lactic acid-glycolic acid enable release of molecules for long periods such as up to or over 100 days, certain hydrogels release compounds for shorter time periods.

As mentioned above, Pheroid® technology is described in for example U.S. Patent Publications No. 20120302442 and 20140194288. The Pheroid® unit consists of an organic carbon backbone composed of unsaturated fatty acids with side-chain interactions, resulting in self-emulsifying characteristics. These vesicles and nano-sponges can entrap hydrophilic, hydrophobic or amphiphilic compounds for biomedical applications and can be modified in terms of characteristics related to loading ability, mechanical resistance, permeability, size and solubility.

The Pheroid® technology allows entrapment of compounds in long-chain fatty acid-based nano- and micro-particles. Particles can be encapsulated, or entrapped, in different media to improve absorption through protecting the contents, and enabling an increase in the level of absorption of the entrapped particles into the bloodstream.

Using the Pheroid® system, green tea extracts as disclosed herein for topical use can be prepared with enhanced tissue absorption, and green tea extracts as disclosed herein for oral use that can better survive passage through the digestive tract and into the bloodstream. Once in the bloodstream, human cells perceive the Pheroid® material as a biological building block and as a source of energy, allowing them to pass through the cell membrane and, by metabolism of the long chain fatty acids, release the phytonutrients directly to the tissues.

There is evidence that for some pharmaceuticals, there may be an advantage to providing them in the form of amorphous crystals, compared to crystallized forms; both are versions of the solid state form of the molecules. For example, Hancock, C. et al., Pharmaceutical Res. 17:397, 2000, reported that the solubility may be improved by providing the pharmaceutical in amorphous crystal form. Kaur, H. et al., J. Pharmaceuticals 2014, Article IS 180845 (2014) reported on methods used to enhance the solubility of polyphenols in the context of drug delivery systems. These publications are incorporated by reference herein for their teachings that may be relevant to pharmaceutical compositions comprising the green tea extracts of the present disclosure. Solubility relates directly to bioavailability, in terms of absorption of drugs into the body. The crystalline to amorphous form of a drug can increase solubility between 10 and 1600 fold.

The solid state form of the molecules is important because of the differences in physical and chemical characteristics. For example, in the crystalline form, generally the molecules are packed in a regularly ordered repeating pattern, providing a thermodynamically stable form. This form also tends to be less soluble. In contrast, in the amorphous form the molecules tend to be in a random arrangement, providing less stability, but also higher solubility. X-ray powder diffraction is one method for determining whether a sample is amorphous or crystalline. A crystalline diffraction pattern shows a pattern of discrete peaks, whereas an amorphous crystalline sample will show no diffraction, and instead a dispersive scatter of X-rays.

Catechin amorphous crystals were observed in a sample processed using the method of Example 1. FIG. 23 is a qualitative confocal micrograph of the extract showing morphology of amorphous crystals with inclusions. Sizes can include ranges such as from about 30 nm to at least 900 nm, and can be larger than 900 nm. The compositions of the disclosure can contain not less than 1% amorphous crystals by weight of total weight of composition. The compositions can contain more than 1% amorphous crystals, such as 2%, 3%, 5% or 10% or more by weight of total weight of composition.

The Examples below are included to demonstrate preferred embodiments of the disclosure. It should be appreciated by those of ordinary skill in the art that the techniques disclosed in the Examples represent techniques and compositions discovered by the inventor to function well in the practice of the disclosure, and thus can be considered to constitute preferred modes for its practice. However, those of ordinary skill in the art should, in light of the present disclosure, appreciate that many changes can be made in the specific embodiments which are disclosed and still obtain a like or similar result without departing from the spirit and scope of the disclosure.

### Examples

### Example 1. Processing Green Tea Plant Material

A non-limiting method of processing the green tea plant material makes use of a High Shear Processor as described in U.S. Pat. No. 6,783,271, the contents of which are incorporated herein by reference. Optimal use of this process is based on the condition of the raw material, for example the green tea leaves. Based on extensive preliminary work, certain guidelines described herein can protect the integrity of the tea catechins prior to processing.

Excess heat and physical conditions can damage newly-picked leaves in the field, so the leaves should be collected and packed with care to ensure that the leaves are not bruised or exposed to temperatures above 28° C. As soon as possible, the leaves are transferred to a temperature controlled environment with temperature below 28° C. and preferably below 25° C. Following transportation as required, the leaves are processed by the method of this example as soon as practical, to ensure that the raw plant material is fresh and living. The freshly picked green tea leaves have been found to tolerate storage in a chiller for 72 hours.

The tea emulsion resulting from the processing contains catechins. The emulsion is further processed for extraction of phytonutrients, specifically catechins in this example, and for optional removal of caffeine, a non-limiting method for which is provided in Example 2.

The method of this Example was used to process tomato tissue. The predominant form of lycopene in tomatoes is the all-trans stereoisomer, which is the straight chain form. The cis/trans ratio of tomato lycopenes was altered in tomato emulsions and powders produced by sono-cavitation of tomato tissue using a High Shear Processor, resulting in a high content of cis-lycopene isomers.

FIG. 23 is a confocal micrograph showing that this process can provide catechin amorphous crystals.

### Example 2. Green Tea Catechin Composition Analysis

This example describes the polyphenol profile of an exemplary catechin composition prepared according to Example 1. The total polyphenols are measured using methods described in Methods of Enzymology 299:152-178 (1999), and the catechins are measured as described in Sakakibara, H. et al., J. Agric. Food Chem. 51:571-581 (2003). The catechins analyzed are as follows: epi-gallocatechin (EGC), catechin (C), epicatechin (EC), epigallocatechin-3-gallate (EGCG), gallocatechin gallate (GCG), epicatechin-3-gallate (ECG), catechin gallate (CG), and gallocatechin (GC). The results are shown in Table 1.

TABLE 1
Catechin composition analysis

| Catechin | Amount, mg/g |
|---|---|
| Epi-gallocatechin (EGC) | 292.0 |
| Catechin (C) | 20.3 |
| Epicatechin (EC) | 56.8 |
| Epigallocatechin-3-gallate (EGCG) | 294.0 |
| Gallocatechin gallate (CCC) | 67.1 |
| Epicatechin-3-gallate (ECG) | 86.9 |
| Catechin gallate (CG) | 12.1 |
| Gallocatechin (GC) | 33.9 |
| (Caffeine) | 118.0 |
| Total Catechins | 863.0 |
| Total Polyphenols (Gallic Acid | 650.0 |
| Equivalents) | |

The percentage of catechins can be increased by further removing caffeine, which can be performed, for example, using a method described in Jour. Food Sci. 48:745-747, 1983.

### Example 3. Green Tea Catechin Chromatographic Analysis by HPLC

An exemplary method is provided in Rinaldo, D., et al., Chirality 22:726-33 (2010), which describes the direct separation of catechin, ent-catechin, epicatechin, and ent-epicatechin in normal phase by HPLC-PAD-CD using Chiralcel OD-H as chiral stationary phase and n-hexane/ethanol with 0.1% of TFA as mobile phase.

### Example 4. Separation of Cis and Trans Catechins in Green Tea Extracts

Another exemplary method for catechin separation is performed using a narrow-bore HPLC procedure to separate cis and trans catechins (catechin and epicatechin). Three different calixarene bonded stationary phases are successfully applied with AcN-2.65 mM H₃PO₄ (10:90, v/v), pH 3.0, as mobile phases to allow a complete separation of six catechins and six xanthines present in aqueous-AcN extracts of green tea. The trans isomer (catechin) is eluted before the cis isomer (epicatechin) on each calixarene-based packing. The distribution profiles of catechins in series of commercially available green teas and catechin-based nutraceuticals can be determined and compared with the distribution profiles of catechins in the green tea extracts of the disclosure to provide detailed comparison data of relevance for the other Examples below.

### Example 5. Extraction of Catechins and HPLC Chromatography to Separate Cis and Trans Catechins

Another exemplary method is provided in Hammerbacher, A. et al. Plant Physiology 164:2107-2122, 2014). This example is performed to analyze the relative ratios of cis and trans catechins, including 2,3-trans-(+)-catechin, 2,3-cis-(−)-epicatechin, 2,3-trans-(+)-gallocatechin, and 2,3-cis-(−)-epigallocatechin. Catechin compounds are extracted from green tea extract material by first grinding the material to a fine powder in liquid nitrogen and lyophilizing at 0.34 millibar pressure using, for example, an Alpha 1-4 LD Plus freeze dryer (Martin Christ GmbH).

About 80 mg dried tissue is then extracted with 2 ml analytical grade methanol for four hours at 4° C., the extract is centrifuged at 3,200 g, and the supernatant is recovered. Insoluble material is reextracted with 1.5 mL methanol for 16 h. Supernatants are combined and evaporated to dryness under a stream of nitrogen. Dried samples are redissolved in 1 mL methanol containing 100 mg mL⁻¹ chlorogenic acid (Sigma) as internal standard. For LC-ESI-mass spectrometry (MS) or hydrolysis of condensed tannins, samples are diluted five times (v/v) with methanol. For LC-FLD, samples are diluted two times (v/v) in acetonitrile.

LC-ESI-MS is performed as follows. Compounds to be analyzed are separated on a Nucleodur Sphinx RP18ec column with dimensions of 250 3 4.6 mm and a particle size of 5 mm (Macherey Nagel) using an Agilent 1100 series HPLC with a flow rate of 1.0 mL min⁻¹. The column temperature is maintained at 25° C. Phenolic

compounds are separated using 0.2% (v/v) formic acid and acetonitrile as mobile phases A and B, respectively, with the following elution profile: 0 to 1 min, 100% A; 1 to 25 min, 0% to 65% B in A; 25 to 28 min, 100% B; and 28 to 32 min, 100% A.

Compound detection and quantification is accomplished with an Esquire 6000 ESI ion trap mass spectrometer (Bruker Daltronics). Flow coming from the column is diverted in a ratio of 4:1 before entering the mass spectrometer electrospray chamber. ESI-MS is operated in negative mode scanning a mass-to-charge ratio (m/z) between 50 and 1,600 with an optimal target mass of 405 m/z. The mass spectrometer is operated using the following specifications: skimmer voltage, 60 V; capillary voltage, 4,200 V; nebulizer pressure, 35 pounds per square inch (psi); drying gas, 11 L min-1; and gas temperature, 330° C. Capillary exit potential is kept at −121 V.

Compounds are identified by MS and by direct comparison with commercial standards, including 2,3-trans-(+)-catechin, 2,3-trans-(+)-gallocatechin, 2,3-cis-(−)-epicatechin, and 2,3-cis-(−)-epigallocatechin. Brucker Daltronics Quant Analysis version 3.4 software can be used for data processing and compound quantification using a standard smoothing width of 3 and Peak Detection Algorithm version 2. Linearity in ionization efficiencies is verified by analyzing serial dilutions of randomly selected samples.

An external calibration curve created by linear regression can be used for quantification of 2,3-trans-(+)-catechin (Sigma) and 2,3-trans-(+)-gallocatechin (Sigma). Process variability in different analyses is calculated relative to the internal standard.

LC-ESI-MS/MS (liquid chromatography-electrospray ionization-tandem mass spectrometry) is performed as follows. Chromatography is performed on an Agilent 1200 HPLC system. Separation is achieved on a 100-3 4.6-mm Kinetex C18 column with particle size of 2.6 mm (Phenomenex, Torrance, Calif. 90501-1430, USA). Formic acid (0.05% [v/v]) in water and acetonitrile are employed as mobile phases A and B, respectively. The elution profile is as follows: 0 to 1 min, 100% A; 1 to 7 min, 0% to 65% B in A; 7 to 8 min, 65% to 100% B in A; 8 to 9 min, 100% B; and 9 to 10 min, 100% A. The total mobile phase flow rate is 1.5 mL min⁻¹. The column temperature is maintained at 25° C.

An API 3200 tandem mass spectrometer (Applied Biosystems) equipped with a turbospray ion source is operated in negative ionization mode. The instrument parameters are optimized by infusion experiments with pure standards of catechin and gallocatechin. The ion spray voltage is maintained at −4,500 V. The turbo gas temperature is set at 700° C. Nebulizing gas is set at 70 psi, curtain gas at 25 psi, heating gas at 60 psi, and collision gas at 10 psi. Multiple reaction monitoring is used to monitor analyte precursor ion→product ion: m/z 299.9→109.1

(collision energy [CE], −34 V; declustering potential [DP], −30 V) for catechin; m/z 304.8→179 (CE, −28 V; DP, −390 V) for gallocatechin; m/z 576.9→289.1 (CE, −30 V; DP, −50 V) for PA B1; m/z 592.9→125.1 (CE, −52 V; DP, −400 V) for the catechin:gallocatechin dimer; and m/z 609→125.1 (CE, −50 V; DP, −45 V) for the gallocatechin dimer. Both Q1 and Q3 quadrupoles are maintained at unit resolution. Analyst 1.5 software (Applied Biosystems) can be used for data acquisition and processing. Linearity of compound detection for quantification is verified by external calibration curves for catechin. Concentrations present in the original extracts are determined relative to the catechin calibration curve.

### Example 6. Pheroid® System for Administration of Green Tea Extract

The relative bioavailability of green tea extract of the disclosure is measured as follows. Green tea extract is incorporated in Pheroid® Pheroid®-green tea extract and green tea extract alone are administered to a human volunteer, and plasma concentration of EGCG and optionally other catechins is measured at regular intervals from 0 to 500 minutes. The results indicate whether administering green tea extract incorporated in Pheroid® increases the plasma concentration compared to green tea extract alone, suggesting enhanced bioavailability.

### Example 7. Parasiticidal Efficacy of Green Tea Extract

The aim of this Example was to determine the in vitro antiplasmodial activity of Phytofare® catechin complex. The test sample was tested in triplicate against a chloroquine sensitive (CQS) strain of Plasmodium falciparum (NF54). Continuous in vitro cultures of asexual erythrocyte stages of Plasmodium falciparum were maintained using a modified method of Trager, W. et al. (Science 193:673-675, 1976). Quantitative assessment of antiplasmodial activity in vitro was determined via the parasite lactate dehydrogenase assay using a modified method described by Makler, M. T. et al., The American Society of Tropical Medicine and Hygeine 48:739-741, 1993.

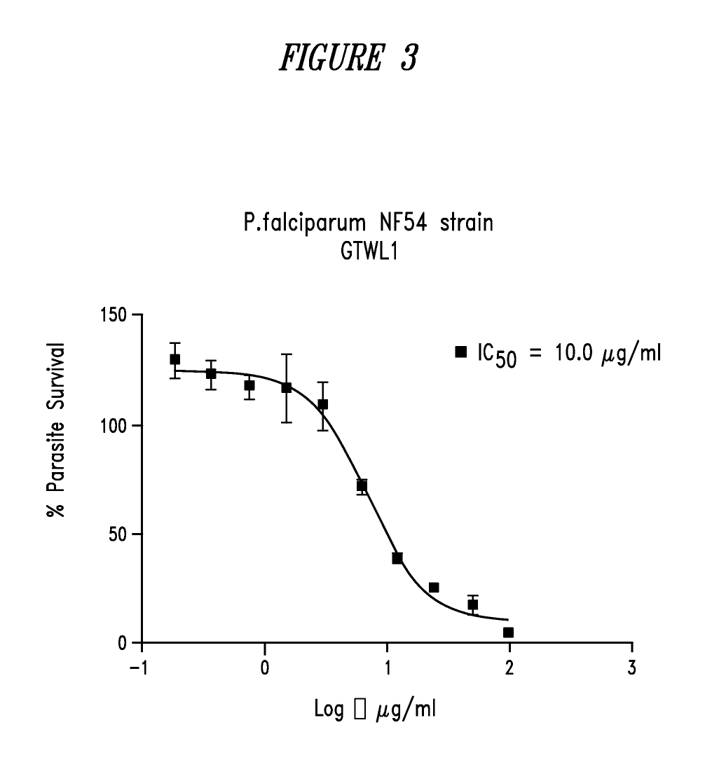

A full dose-response experiment was performed to determine the concentration inhibiting 50% of parasite growth (IC₅₀-value). The IC₅₀-values were obtained using a non-linear dose-response curve fitting analysis via Graph Pad Prism v4.0 software and are shown in FIG. 3.

The results showed that Phytofare® catechin complex (GTWL) was active against NF54 Plasmodium falciparum strain with an IC₅₀-value of 10 μg/ml.

### Example 8. Parasiticidal Efficacy of Phytofare®

This example was carried out to determine the parasiticidal efficacy of Phytofare® green tea extract containing catechins. The extract was resuspended in water to give a stock concentration of 58 mg/ml. Parasite growth was measured by hypoxanthine uptake using radiolabeled hypoxanthine as described (Slavic, K. et al., Mol. Biochem. Parasitol. 168:113-116, 2009). The concentration range for the IC₅₀ assay was 580 μg/ml to 1.13 μg/ml, serially diluted 2-fold. Each experiment included five technical replicates, and a total of three biological replicates were carried out.

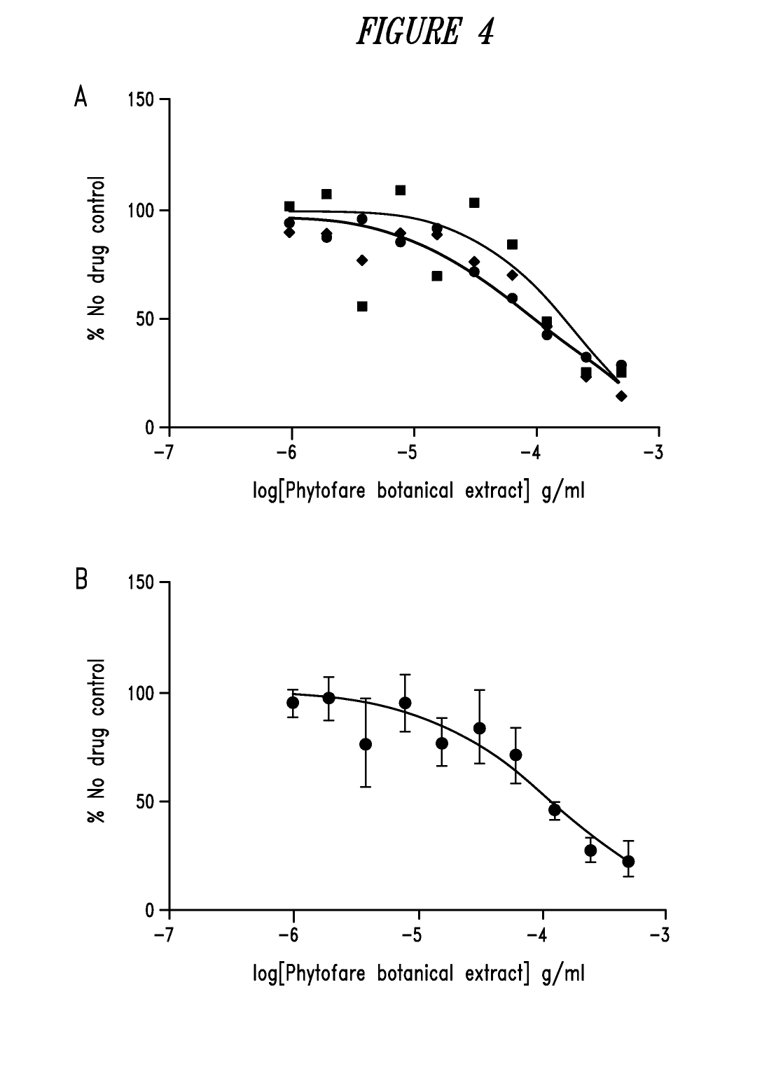

The bottom graph of FIG. 4 shows an IC₅₀ of 118.0 μg/ml (±1.19) for the extract. The molar equivalents of the various catechins in the extract at this concentration is shown in the table below.

| | | @ 580 | μM @ 580 | μM @ IC50 |
|---|---|---|---|---|
| Analysis | Result | μg/ml | μg/ml | value 118 μg/ml |
| Caffeine | | | | |
| Caffeine | 118 mg/g | 68.44 | 352.44 | 71.70 |
| Catechin | | | | |
| Epigallocatechin | 292 mg/g | 169.36 | 552.98 | 112.50 |
| Catechin | 20.3 mg/g | 11.77 | 40.56 | 8.25 |
| Epicatechin | 56.8 mg/g | 32.94 | 113.49 | 23.09 |
| Epigallocatechin | 294 mg/g | 170.52 | 372.01 | 75.68 |
| Gallate | | | | |
| Gallocatechin | 67.1 mg/g | 38.92 | 84.91 | 17.27 |
| Gallate | | | | |
| Epicatechin Gallate | 86.9 mg/g | 50.40 | 113.94 | 23.18 |
| Catechin Gallate | 12.1 mg/g | 7.02 | 15.86 | 3.23 |
| Gallocatechin | 33.9 mg/g | 19.66 | 64.20 | 13.06 |
| Total Catechins | 863 mg/g | 500.54 | 1357.95 | |
| Total | 650 mg/g | 377.00 | | |
| Polyphenols(Gallic | | | | |
| Acid Equivalents) | | | | |

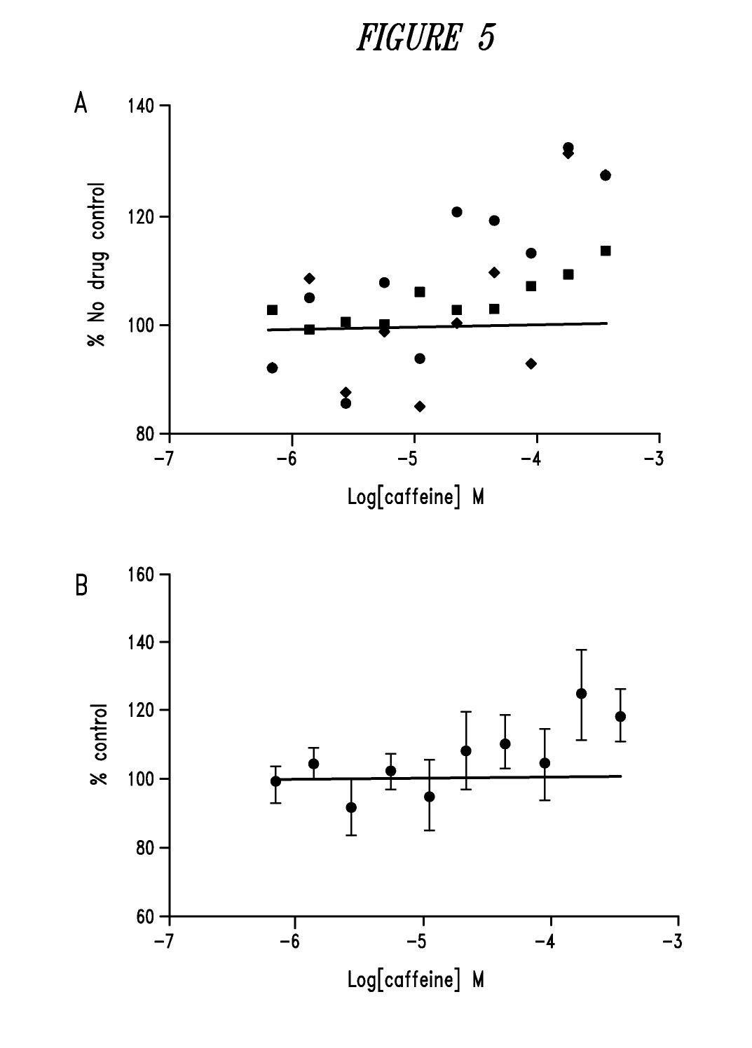

The assay was repeated to obtain the IC₅₀ values for pure caffeine, and for Epigallocatechin Gallate (EGCG) which is present in the extract, obtained from a different source. The concentration ranges used were 352.44 μM to 0.69 μM for caffeine, and 371.01 μM to 0.73 μM for EGCG. These concentrations reflect the molar concentrations present at the highest concentration (μg/ml) of extract used. At the IC₅₀ for the extract, the concentration of caffeine was 71.70 μM. FIG. 5 shows that there was no inhibition by caffeine alone even at 352.44 μM. The concentration of EGCG at the IC₅₀ for the extract was 75.68 μM. The IC₅₀ for EGCG alone was 80.78 μM±1.11, which is higher than the EGCG concentration in the green tea extract and is comparable to that of commercial purified EGCG. In conclusion for Example 8, anti-malarial activity of EGCG in the extract has been documented, and an effect of caffeine has been ruled out.

### Example 9. Anti-Malarial Action of Gallate Catechins

Fatty acid biosynthesis by the malaria parasite involves an enoyl-acyl carrier protein reductase. Preliminary research suggests that gallate catechins inhibit this enzyme, thereby interfering with fatty acid biosynthesis in the parasites.

Green tea catechins have an inhibitory effect on Plasmodium falciparum hexose transporters. Using methods such as those described in Slavic, K. et al. (Mol. Biochem. Parasitol. 168:113-116, 2009), green tea extracts of this disclosure are tested for their effect on hexose transporters.

### Example 10. Effects of Green Tea Extract-Containing Compositions on Parameters of Skin Aging

This Example was conducted to compare the effects of two preparations, one containing a green tea extract of the disclosure (“Formula A”) and the other a control (“Formula B”) on the skin moisture content, elasticity, and surface parameters of human skin at 14, 28 and 42 days after the daily application of both the formulas. Formula A was a base cream formulated with green tea extract as described in this disclosure. Formula B was a control, and consisted of the base cream without the green tea extract. The Table below shows the components in each composition.

| Component in test composition (A) | Component in control composition (B) |
|---|---|
| Pheroid vesicles | — |
| Green tea extract (active | — |
| component) | |
| ProPheroid ™ | H₂O |
| Cetyl Alcohol | Cetyl Alcohol |
| Oleic Acid | Oleic Acid |
| Cremaphor RH40 | Cremaphor RH40 |
| Isopropyl meristat | Isopropyl meristat |
| Beeswax | Beeswax |
| Methyl Paraben | Methyl Paraben |
| Propyl Paraben | Propyl Paraben |
| BHA | BHA |
| BHT | BHT |
| Rosemary essential oil | Rosemary essential oil |

The study was carried out on the inner volar aspect of the non-dominant forearm of the thirty five healthy Caucasian female volunteers aged from 35 to 65 years. The washout period started seven days before the study commenced. During the washout and study periods, the volunteers used only Dove soap and no other skin care products.

A recording area of (2 cm×6 cm=12 cm²) was marked on the non-dominant inner forearm of the volunteers. On day 1 of the study (T0days), the pre-application recordings were performed using a Corneometer® (skin moisture content), a Cutometer® (skin elasticity) and a Visioscan® VC 98 camera (skin surface parameters) on the recording skin area. (All instruments are produced by Courage+Khazaka Electronic GmbH, Cologne, Germany.)

Following the T0days recordings, Formula A was applied (2 mg/cm²) to the recording area twice daily and the skin parameters were recorded 14 days (T14 days), 28 days (T28 days) and 42 days (T42 days) post application. After a washout period of two months and a pre-application recording (T0days), Formula B was applied to the recording area on the skin of the same volunteers as for Formula A. The skin parameters were recorded post application of Formula B at the same time intervals as for Formula A. The volunteers had strict instructions not to apply any other products

### Skin Hydration

The Corneometer® is worldwide the most utilized instrument to determine the hydration level of the skin surface, mainly the stratum corneum. The measurement is based on the capacitance measurement of a dielectric medium. The Corneometer® measures the change in the dielectric constant due to skin surface hydration, changing the capacitance of a precision capacitor. An increase in the Corneometer® reading indicates an increase in stratum corneum hydration.

The Corneometer® readings were taken at T0days before treatment started and at T14 days, T28 days and T42 days, after treatment with Formulas A and B respectively. The Corneometer® readings at the various time intervals were corrected for the base line readings at T0days. The changes in the Corneometer® readings expressed as a percentage of the base line readings were calculated as a function of the time after application of Formulas A and B respectively.

The percent change in skin hydration as a function of the time after application of Formulas A and B is shown in the Table below:

Percent Change in Skin Moisture as a Function of Time after Application

| Composition | T14 Days | T28 Days | T42 Days |
|---|---|---|---|
| Formula A (Green tea | 9.42 | 15.17 | 15.11 |
| extract) | | | |
| Formula B (control) | 7.83 | 0.90 | 7.84 |

Both formulas caused the skin to be more hydrated when compared to the moisture content of the pre-treated skin at all the recording intervals. However, there were noticeable differences in the magnitude of the hydration effects of the two formulas at the various recording intervals.

To establish whether the hydration effects of formulas A and B were statistical significantly different, the baseline corrected hydration responses were subjected to Student's t-test statistical analysis utilizing a 95% confidence interval (p<0.05). Although Formula A performed better than Formula B at all recording intervals in terms of its hydrating effect, its performance was only statistical significantly (p=0.01) better (15%) than that of formula B (0.9%), 28 days post application.

Formula B lost some of its hydrating effect after 28 days post application but regained its effect 42 days post application. The effect of Formula B at 42 days was comparable to the effect 14 days post application. This cyclic phenomenon is sometimes seen when the skin re-adjusts itself following treatment with formulas that influence the barrier function of the skin indirectly. Formula A maintained its hydrating effect up to 42 days post application and the effect was better than for Formula B although not statistically significant.

In conclusion to the skin moisture experiment, both Formulas A and B hydrated the skin at all recording time intervals and can be regarded as moisturizers. The hydrating effect of Formula A was superior to that of Formula B at all recording time intervals but this effect was statistical significant only 28 days post application. Formula A when compared to Formula B showed optimum hydrating effect at 28 days post application and maintained the effect up to 42 days

### Skin Surface Parameters

The process of aging has a direct impact on the status of the skin surface. To evaluate the impact of anti-aging formulas on the skin, it is necessary to characterize the skin surface qualitatively and quantitatively. This was achieved by means of the Visioscan® VC 98, a UVA-light camera with a black-and-white video sensor that records, under uniform illumination, high resolution images of the skin.

For this example, the camera was connected to a computer by means of a digitalization unit (Video Digitizer VD 300) via a FireWire port. The images were displayed in 256 grey levels which are then transferred as graded grey values. Wrinkles appeared dark in the image and scaliness was shown in bright pixels. Multi-functional software, which utilizes the SELS (Surface Evaluation of the Living Skin) method of evaluation, analyzed the grey level distribution and allowed the calculation of clinical parameters to quantitatively and qualitatively describe the skin surface with parameters such as Skin roughness (Ser) and Scaliness (Sesc).

### Skin Roughness (Ser)

Ser is the roughness parameter which calculated the proportion of dark pixels and recorded discontinuous areas within the stratum disjunctum, especially close to wrinkles and the lines of the skin surface. The smaller this value is, the less rough the skin is.

The Ser parameter was calculated from the images recorded at T0days before treatment started and at T14 days, T28 days and T42 days, after treatment with Formula A and B respectively. The Ser values calculated at the various time intervals were corrected for the Ser values calculated from the base line images recorded at T0days. The changes in the Ser values, expressed as a percentage of the base line Ser values, were calculated as a function of the time after application of Formulas A and B respectively.

The percent change in skin roughness as a function of the time after application of Formulas A and B respectively, is shown in the table below:

Percent Change in Skin Roughness as a Function of Time after Application

| Composition | T14 Days | T28 Days | T42 Days |
|---|---|---|---|
| Formula A (Green tea | −5.56 | −14.60 | −10.56 |
| extract) | | | |
| Formula B (control) | 12.67 | 5.80 | 8.68 |

Formula A reduced the roughness of the skin at all recording time intervals post-application. This effect was most prominent 28 days post application where Formula A reduced the skin roughness by close to 15% as compared to untreated skin. The effect after 42 days was slightly less (11%) than at 28 days.

In contrast, Formula B tended to increase the roughness at all recording intervals. This effect was the lowest (6%), 28 days post application. At 42 days the effect was lower (9%) than at 14 days (13%) post application. Formula A performed statistically significantly (Student's t-test, p<0.05) better than Formula B in reducing skin roughness after 14 days (p=0.03), 28 days (p=0.007) and 42 days (p=0.03) post application. Formula B had an opposite effect than Formula A in reducing skin roughness.

In conclusion to this experiment on skin roughness, Formula A reduced while Formula B enhanced skin roughness at all recording intervals post application. The difference in the effects of Formulas A and B on skin roughness was statistically significant at all recording intervals post application. The maximum (15%) reduction in skin roughness by Formula A was achieved 28 days post application, whereafter the effect diminished by 4%, 42 days after application.

Skin Scaliness

Sesc is the skin scaliness parameter and calculates the portion of the bright pixels which registers the partially or almost completely detached areas of the stratum disjunctum referred to as scales. The smaller the value of Sesc the less scaliness there is on the stratum corneum and corresponds with higher skin moisture.

The Sesc parameter was calculated from the images recorded at T0days before treatment started and at T14 days, T28 days and T42 days, after treatment with Formulas A and B respectively. The Sesc values calculated at the various time intervals were corrected for the Sesc values calculated from the base line images recorded at T0days. The changes in the Sesc values expressed as a percentage of the base line Sesc values were calculated as a function of the time after application of Formulas A and B respectively. The percent change in skin scaliness as a function of the time after application of Formulas A and B respectively, is shown in the Table below:

Percent Change in Skin Scaliness as a Function of Time after Application

| Composition | T14 Days | T28 Days | T42 Days |
|---|---|---|---|
| Formula A (Green tea | −18.649 | −20.360 | −19.293 |
| extract) | | | |
| Formula B (control) | 1.386 | −8.087 | −7.627 |

The skin scaliness was reduced at all recording intervals after application of Formula A. The maximum reduction (20%) in scaliness was achieved 28 days post application of Formula A where after the effect was slightly less (19%), 42 days post application.

Formula B reduced the scaliness (8%), 28 days after application where after the effect was maintained up to 42 days post application. The effect of Formula A in reducing the scaliness of the stratum corneum was statistically significantly (Student's t-test, p<0.05) better that that of Formula B, 14 days (p=0.001), 28 days (p=0.011) and 42 days (p=0.006) post application of the Formulas.

In conclusion for this experiment on skin scaliness, Formula A reduced the skin scaliness at all recording intervals with the maximum effect recorded 28 days post application and maintained the effect up to 42 days post application. When compared to Formula A, Formula B had less effect on the reduction of scaliness. The difference in the effects of Formulas A and B on the reduction of skin scaliness was statistically significant at all post application recording intervals.

### Skin Elasticity

The skin behaves like a complex substrate possessing elastic, viscous and plastic properties. The elastic properties reflect the skin's ability to return to its initial position after deformation and can be affected by chronological- and photo aging of the skin. Loss of elasticity is a prominent feature of aging skin. Skin elasticity was evaluated by means of a Cutometer®. The measuring principle of the Cutometer® is based on a suction method that consists of the measurement of vertical deformation of the skin surface after application of a vacuum. A defined negative air pressure (350 mbar) is applied perpendicular to the skin through the opening of the probe for a selected time period (5 sec.).

In this example, the skin surface to be evaluated was sucked into the aperture (2 mm) of the probe, and the resulting vertical deformation was measured by the optical measuring system inside the probe. The changes of light intensity were proportionally related to the penetration depth of the skin and were displayed on a computer monitor as curves in a coordinate system as skin deformation (mm) versus time (sec.).

From standard deformation graph data, various R parameters were calculated by the Cutometer® software. The relative parameter R2, was selected for the purpose of this example to evaluate the gross elasticity of the skin and the parameter is defined as the ratio between the total recovery (Ua) and the final deformation (Uf). The closer this value is to 1 the more elastic the skin is. The R2 parameter is a relative parameter in that it gives the ratio of two primary parameters, Ua and Uf, and is considered to be independent of skin thickness, and its value in different subjects, anatomical regions and times can be compared.

The elasticity parameter, R2, was calculated from the deformation graph data recorded at T0days before treatment started and at T14 days, T28 days and T42 days after treatment with Formulas A and B respectively. The R2 values calculated at the various time intervals were corrected for the R2 values calculated from the base line graph recorded at T0days. The changes in the R2 values expressed as a percentage of the base line R2 values were calculated as a function of the time after application of Formulas A and B respectively. The percent change in skin elasticity as a function of the time after application of Formulas A and B respectively, is shown in the Table below:

Percent Change in Skin Elasticity as a Function of Time after Application

| Composition | T14 Days | T28 Days | T42 Days |
|---|---|---|---|
| Formula A (Green tea | −0.750 | 0.213 | 1.098 |
| extract) | | | |
| Formula B (control) | −0.77 | −1.03 | 0.10 |

Both Formulas A and B reduced the skin elasticity 14 days after application with no statistically significant (Student's t-test, p<0.05) differences between the effects of Formulas A and B. However, after 28 days post application, Formula A enhanced the elasticity reaching a maximum 42 days post application. Formula B did not enhance the elasticity and its effect at 28 days was comparable to the effect at 14 days showing a decrease in elasticity. After 42 days of application the effect on elasticity was close to zero.

A large variation in the response to the effects of Formulas A and B on skin elasticity was noted. There was no obvious reason for the variation other than that the magnitude of the effect on elasticity is too small to discriminate between normal biological variation without any intervention and the effects caused by either one of the Formulas.

In conclusion to this experiment on skin elasticity, Formula A showed a trend towards enhancing skin elasticity increasing from 14 days to 42 days post application. Formula B had no significant effect on enhancing skin elasticity and the trend, moving from 14 days to 42 days post application, was in a negative direction. Due to the small observed effect, and the inter subject variation, the differences in the effects of Formulas A and B were not statistically significant (p values >0.05).

Summary for Example 10.

As intrinsic (chronological) skin aging occurs, the skin acquires more wrinkles, becomes drier, and loses its elasticity. Skin roughness and scaliness are significantly increased in the aged. Therefore, skin formulations that moisturise the skin, reduce skin roughness and scaliness as well as enhancing skin elasticity can be regarded as having anti-aging properties. Wrinkle levels per se were not evaluated, because a decrease in elasticity is regarded as the variable that determines wrinkle levels. The evaluation of elasticity is therefore more important because it is not as visible as wrinkles as a sign of aging.

Green tea extract-containing Formula A moisturized the skin, reduced skin roughness and scaliness as well as having some trend towards enhancing skin elasticity. Formula B (control) moisturized the skin and reduced scaliness, but surprisingly had an increased effect on skin roughness and very little effect on elasticity. The effects of both Formulas A and B on skin elasticity were small and not significantly different from each other although Formula A showed a promising trend after 42 days post application. The small effects as a function of treatment time may be an indication that longer treatment is needed to influence the complex biophysical mechanisms involved in changing skin elasticity.

A large inter subject variation was observed for all of the parameters evaluated. As the power of a statistical test is influenced by the magnitude of the effect and the sample size used to detect the effect, the statistical power might have been not sufficient in the case of the elasticity parameter to discriminate between the effects of Formulas A and B. In this case an increase in sample size will assist in discriminating between the effects of the two formulas on skin elasticity. Although both formulas affected the evaluated parameters quantitatively, the effects of Formula A were superior to that of Formula B in magnitude and duration. For all the parameters, except for the elasticity parameter, the maximum effects were seen 28 days after treatment and lasted with slight variance up to 42 days post treatment.

In conclusion, this Example shows the following: in comparison to the control Formula B, the green tea extract-containing composition of Formula A had prominent positive effects on skin hydration and skin surface parameters. Formula A therefore is regarded as having anti-aging properties superior to the control Formula B that did not contain green tea extract.

### Example 11. Treatment of Actinic Keratosis

The present disclosure provides for the use of a pharmaceutical formulation containing green tea extract as disclosed above in an amount of about 10% (w/w) to about 15% (w/w) in the pharmaceutical formulation for the treatment of actinic keratosis, solar keratosis and/or basal cell carcinoma. The mixture of different polyphenols contains in particular more than 60% (w/w), especially more than 65% (w/w) gallates of catechol, epicatechol, epigallocatechol or of gallocatechol.

A preferred mixture of different polyphenols is Phytofare® as specified above. One preferred pharmaceutical formulation comprises about 35% (w/w) of isopropyl myristate, about 15% (w/w) of Phytofare® Pheroid®, about 24.5% (w/w) of petroleum jelly, about 20% (w/w) of wax, about 5% (w/w) of propylene glycol monostearate or propylene glycol monopalmitostearate and about 0.5% (w/w) of oleyl alcohol for use in the treatment of actinic keratosis, solar keratosis and/or basal cell carcinoma.

Patients diagnosed with actinic keratoses are treated with Phytofare® (15% ointment containing 35% (w/w) isopropyl myristate, 15% (w/w) catechol extract, 24.5% (w/w) petroleum jelly, 20% (w/w) wax, 5% (w/w) propylene glycol monostearate and 0.5% (w/w) oleyl alcohol). The treated area is for example about 5 cm² on the forehead, at a treatment schedule of five times a week (each with ten hours) over a treatment period of six weeks.

After about thirteen days of treatment, skin irritation of the treated area (especially, treated areas afflicted by actinic keratoses) is evaluated, and during further treatment after twelve weeks of treatment the alleviation and disappearance of actinic keratoses is evaluated.

### Example 12. Bioavailability of Green Tea Extract in Overweight Humans

This example is performed to evaluate the bioavailability of green tea extract of the disclosure in comparison with one, two or more commercially available green tea products. The study involves twelve subjects (six males, six females) between 18 and 65 years of age, selected with the following criteria: body mass index of 25-29.9 kg/m²; stable weight; agreement to maintain current level of physical activity during study; and not excluded due to use of prescription or over the counter drugs, or having an allergy to one or more ingredients in the products to be studied.

Prior to starting the study, the height, weight, BMI, heart rate, and blood pressure are measured and recorded for each subject. Subjects are randomized to a treatment group, and begin the study after fasting for 12 hours, at which time fasting blood is taken.

Subjects receive a dose of one of the study compositions (time=0), and blood is collected at regular intervals, such as at 15, 30, and 45 minutes, and 1, 2, 4, and 8 hours post-dose. The levels of Epigallocatechin, Catechin Epicatechin, Epigallocatechin Gallate, Gallocatechin Gallate, Epicatechin Gallate, Catechin Gallate, Gallocatechin, and total Catechins are analyzed.

Following a washout period, such as one week, the subjects undergo the same process of dose administration and blood collection, with administration of a different one of the three treatments.

The levels of individual catechins and total catechins is tabulated and correlated with treatment group to reach conclusions about the relative value of the different treatments as measured by blood levels of catechins after time=0 and the duration of the change in blood levels.

### Example 13. Effect of Green Tea Extract on Weight Loss

This example is performed to evaluate the effect of a green tea extract of the disclosure on weight loss, and is performed as a randomized, double-blind, placebo controlled parallel study of ninety human subjects, thirty per test group. After a screening process to identify eligible individuals based on medical condition, medical history, and other parameters, subjects undergo baseline measurements of weight, waist circumference, hip circumference, arm circumference, thigh circumference, heart rate, and blood pressure. The BMI and waist/hip ratio are calculated, and resting metabolic rate (RMR) is recorded.

Fasting blood is collected to determine a lipid panel (LDL-C, TC, TG, HDL-C) and glucose. Blood is also drawn to analyze hsCRP, adiponectin, testosterone, leptin, total antioxidant capacity, bHB, and NEFA. A DEXA Scan is performed. The subjects are randomized to a treatment group. Investigational product and treatment diaries are dispensed, with instructions on use. The treatment diaries are used to record daily treatment product use, changes in therapies, and any adverse effects.

At regular intervals after starting the treatment, such as day 14, day 28, and day 56, measurements of weight, waist circumference, hip circumference, arm circumference, thigh circumference, heart rate, and blood pressure are taken, and the BMI and waist/hip ratio are calculated. Fasting blood is collected to determine a lipid panel and blood glucose. The treatment diaries are reviewed for compliance.

At the end of the study, such as day 84, the body measurements are repeated, and the heart rate and blood pressure are taken. BMI and waist/hip ratio are calculated, and resting metabolic rate is taken. Fasting blood is collected for a lipid panel, and blood is drawn to analyze hsCRP, adiponectin, testosterone, leptin, total antioxidant capacity, bHB, and NEFA. A DEXA Scan is performed. Blood is also analzed for CBC, electrolytes (Na, K, Cl), glucose, creatinine, AST, ALT, GGT, and bilirubin.

To measure the effect on weight loss during the study, the primary endpoints that will be correlated with the treatments are weight loss and lipid panel. The secondary endpoints are fasting glucose; hsCRP; Adiponectin; antioxidant status (NEFA, bHB, total antioxidant status); and hormones leptin and testosterone. Conclusions will be drawn as to the relative effect of the treatments on these parameters of weight loss.

### Example 14. Lipase Inhibition Test

This example is performed to determine the effect of green tea extracts of the disclosure on biochemical parameters associated with metabolism and absorption of fat, which play a role in obesity. Reference standards and organic solvents (ACS or HPLC grade) can be obtained from Sigma Aldrich (Vienna, Austria). Standard laboratory chemicals are p.a grade. A lipase test kit can be obtained from Trinity Biotech (Jamestown, N.Y., USA, Cat No.: 805).

Sample Preparation. Green tea extracts or catechin reference standards, such as EGCG, are suspended in 100 ml acetone/water=6/4 and stirred for 12 hours at room temperature for extraction of polyphenols. Following centrifugation at 5,000 rpm for 10 minutes, the clear supernatant is evaporated to dryness under reduced pressure at 45° C. The dry residue is used for the experiments in this example.

The lipase activity test is performed as follows. Dry residue samples of green tea extract and optionally catechin standards are dissolved in 100 ml CH₃OH/H₂O (1/1 v/v), centrifuged (5,000 rpm for 5 min.) and cleared by filtration (syringe filter 20 μM). Control experiments are performed using the solvent.

Lipase activity is determined using a commercially available test kit. Aliquots of LPS standard, solvent or samples are added to 500 μl of substrate solution, mixed gently and incubated for 5 min at 37° C. After addition of an activator reagent the change in the absorbance rate is followed at 550 nm for 10 minutes. The rate of activity is given as percent of the activity of Lipase PS (labeled with 327 IU/L) obtained from control samples.

Inhibition of chicken liver fatty acid synthase is measured to determine the effect of green tea extracts of the disclosure. The following reagents are obtained commercially, such as from the indicated suppliers: NaH₂PO₄ (Merck A168646), Glycerol (Sigma G5516), NaOH (Aldrich 22, 146-5) Sephadex G-50 (Sigma G-50-80), Ethylendiaminetetraacetic acid EDTA (Sigma E9884), Dithithreiol DTT (Sigma D-9779), polyethylene glycol 6.000 (Fluka-81255), DEAE-cellulose (Fluka 30477), Nicotinamide adenine di-nucleotide-phosphate NADPH₂ (Sigma N-7505), malonyl-CoA (Sigma M-4263), acetyl-CoA-(Sigma A-2056), Total Protein Kit (Sigma TP02000, Bovine Albumin (Fluka 05473).

Extraction of FAS from chicken liver is performed as follows. Liver from young chicken (0.5 kg BW (body weight)) is excised immediately after the animals are sacrificed and stored on ice until further processing. 10 g of minced liver is homogenized in 100 ml ice-cold buffer (0.1 M NaH₂PO₄-buffer with 20% glycerol, pH adjusted to 7.5 with NaOH) using a mechanical homogenizer. The homogenate is centrifuged at 4° C. for 15 min. at 30,000 g.

The resulting supernatant (liberated from the fat layer) is immediately further processed by gel filtration over Sephadex G-50. The gel filtration is performed using 100 ml cartridges from Pharmacia filled with Sephadex G-50 suspended in water. The flow rate is set to approximately 4 ml/min. The elution is followed by UV-detection set to 214 nm. The mobile phase consists of 0.1 M NaH₂PO₄ buffer, 45 mM glycerol, 1 mM EDTA, 1 mM DTT, pH adjusted to 7.5 with NaOH. The first peak corresponding to the protein fraction is collected and pooled (40 ml volume).

The protein fraction is made up to 5% (m/v) polyethyleneglycol and stirred for 30 min. at 4° C. The precipitate is separated by centrifugation (9,000 g, 30 min, 4° C.) and the supernatant is brought to 12% concentration with polyethyleneglycol. The resulting precipitate is collected by centrifugation (9,000 g, 30 min. 4° C.) and used for further processing.

The pellet is carefully washed and then dissolved in 5 ml 0.1 M NaH₂PO₄ buffer, 45 mM glycerol, 1 mM EDTA, 1 mM DTT, pH adjusted to 7.5 with NaOH. This solution is filtered if necessary and stored at −20° C. without loss of activity for 4 weeks.

As a last purification step performed immediately prior to the assay, the resulting solution is purified by ion-exchange chromatography with DEAE-cellulose. In this case glass-pipettes are filled with a 1 ml volume of DEAE-cellulose and equilibrated with 0.1 M NaH₂PO₄ buffer, 45 mM glycerol, 1 mM EDTA, 1 mM DTT, pH adjusted to 7.5 with NaOH. 0.5 ml of the protein solution is loaded onto the column and eluted by step-wise addition of 0.5 ml portions of the same buffer. The fractions eluting between 1.5-2.5 ml are collected and used for the FAS-assay.

The FAS Test-Assay is performed as follows: 150 μl of the purified extract is mixed with 100 μl NADPH₂/Ac-CoA (2.5/0.8 mg/2 ml water corresponding to a final concentration of 150 and 50 μM, respectively). A solution of green tea extract, prepared as described above, is added in volumes of up to 250 μl.

The test assay is then filled up to 1 ml with 0.1 M NaH₂PO₄ buffer, 45 mM glycerol, 1 mM EDTA, 1 mM DTT, pH adjusted to 7.5 with NaOH. After a 30 min. pre-incubation period at 25° C., 100 μl malonyl-CoA (1 mg/2 ml water corresponding to 55 μM) are added as a reaction starter. The reaction is monitored with an UV/VIS spectrometer (Jasco V-530) set to 340 nm against air at 30° C. The activity of the enzyme is calculated from the decrease of absorption which is due to consumption of NADPH₂. The measurement time is set to 15 min.

Gallic Acid, Catechin and Epicatechin are optionally used as reference standards. Calibration solutions are prepared by dissolution of 25 mg standard in 0.5 ml of methanol and dilution with water to 10 ml. Further dilutions are prepared by addition of water. The linear relationship of the method is established between 0.6 mg/10 ml to 24.0 mg/10 ml for all 3 reference compounds.

The stability of Gallic Acid, Catechin and Epicatechin under the extraction conditions is determined. Recoveries of all three standards after methanolic/aqueous extraction and methanolic/acidic extraction are expected to be higher than 95%. For protein determination, protein is determined according to a modified Micro-Lowry method (Total Protein Kit, Micro Lowry, Onishi & Barr Modification) according to the manual. As calibration range 0.15-1.0 mg protein/ml is used. The method is calibrated with bovine albumin.

The activity of the FAS is calculated from the consumption of NADPH expressed as μM×L¹×min¹ using the absorption data obtained and a molar extinction coefficient of 6.3.

### Example 15. α-Amylase Assay

This example is performed to measure the effects of green tea extracts of the disclosure on pancreatic amylase activity in vitro as an indicator of amylase-mediated starch digestion in vivo. Starch is primarily metabolized by α-amylase resulting in the formation of glucose and maltose. Inhibition of α-amylase by green tea extracts and optionally by catechin standards, such as EGCG and EGC, is examined in vitro using a modified version of the chromogenic Red-starch method (Megazyme, Wicklow, Ireland).

Inhibition studies are conducted by combining enzyme (0.3 U/mL) suspended in 20 mM phosphate buffer (pH 6.9) containing 6.7 mM sodium chloride and Red-starch (7 mg/mL in 0.5 M potassium chloride). Green tea extract, or standards such as EGCG and EGC, is added (0-200 μM). Following incubation at 37° C. for 10 min, the reaction is terminated by adding 95% ethanol.

The solution is brought to room temperature, and then centrifuged at 1000×g for 10 min. The absorbance of the supernatant is measured at 510 nm using a Beckman DU650 spectrophotometer. A previous study (Forester, S. C. et al., Mol. Nutr. Food Res. 56:1647-1654, 2012) reported that EGCG and ECG both inhibited α-amylase using this assay, so results from the present example can be compared.

### Example 16. Effect of Green Tea Extracts on Diabetes-Related Markers

7 week-old db/db mice are randomized and assigned to receive diets supplemented with or without green tea extracts for ten weeks. Fasting blood glucose, body weight and food intake are measured during the treatment. Glucose and insulin levels are determined during an oral glucose tolerance test after 10 weeks of treatment. Pancreas tissue is sampled at the end of the study for histomorphometric analysis. Islets are isolated and their mRNA expression analyzed by quantitative RT-PCR.

Ortsäter, H. et al. (Nutrition and Metabolism 9:1-10, 2012) used this method to test EGCG, and found that in db/db mice, EGCG improved glucose tolerance and increased glucose-stimulated insulin secretion. EGCG supplementation reduced the number of pathologically changed islets of Langerhans, increased the number and the size of islets, and heightened pancreatic endocrine area. The authors also reported a reduction in islet endoplasmic reticulum stress markers, which they suggest is possibly linked to the antioxidative capacity of EGCG.

In Orsäter's study the green tea extract EGCG preserved islet structure and enhanced glucose tolerance in genetically diabetic mice, outcomes with relevance to the prevention and treatment of type 2 diabetes using the disclosed green tea extracts.

### Example 17. Effect of Green Tea Extracts on Glucose Tolerance in Genetically Diabetic Mice

This example is performed to evaluate the effect of green tea extracts on preserving islet structure and enhancing glucose tolerance in genetically diabetic mice. These parameters are relevant to nutritional strategies for the prevention and treatment of type 2 diabetes.

Male (7 weeks old) db/db mice are obtained commercially, such as from The Jackson Laboratories (Bar Harbor, Me. USA). For this example, animals are maintained on a 12 h light (300 Lux) and 12 h dark cycle at a humidity of 55-60% and a temperature of 23±1° C. All animals receive modified AIN-93 diets (Provimi Kliba A G, Kaiseraugst, Switzerland; Reeves, P. G., J. Nutr. 127:838S-841S, 1997) and water ad libitum.

The effect of green tea extracts of the disclosure can be compared with commercial green tea products and with one or more catechin standards. For example, dietary EGCG (Teavigo™, DSM Nutritional Products Ltd, Basel, Switzerland) can be used for comparative purposes. Teavigo™ is a highly purified extract from green tea leaves (Camellia sinensis) containing >94% EGCG, <5% other catechins (<3% epicatechin gallate).

Mice are randomized to receive placebo diet, a modified AIN-93 diet containing green tea extracts of the disclosure; containing EGCG at a concentration of 10 g/kg of diet (EGCG 1% [w/w]); containing one or more green tea catechin standards; or containing the thiazolidinedione rosiglitazone (Avandia™ GlaxoSmithKline, Brentford, UK) at a concentration of 21 mg/kg of diet (Rosi 0.0021% [w/w]) for 10 weeks.

Fasting (2 hour) blood glucose levels are measured at 0, 5 and 10 weeks; food intake and body weight are monitored at regular intervals, such as at 0, 3, 6 and 9 weeks. After 10 weeks of dietary treatment, an oral glucose tolerance test (OGTT) is performed. Before application of an oral glucose load (1 g/kg, Sigma, St. Louis, Mo.), blood glucose levels are determined in food-deprived animals (Glucotrend, Roche Diagnostics, Basel, Switzerland). Plasma insulin is determined by use of an ELISA kit (Mercodia AB, Uppsala, Sweden). Insulin resistance can be assessed by either homeostasis model assessment-estimated insulin resistance (HOMA-IR)=fasting glucose (mM)*fasting insulin (μU/ml)/22.5, or by quantitative insulin sensitivity check (QUICKI)=1/[log ((fasting glucose (mg/dl)+(fasting insulin (μU/mI))]. After the OGTT, animals can be sacrificed and pancreas taken for further experiments.

The pancreas of each animal is carefully dissected, a small part of the splenic part of the gland is weighed and placed in acid ethanol (0.18 M HCl in 95% ethanol) to determine insulin content and the rest is immersed in formalin solution and stored at 4° C. until further processing for histological examination. Preparations are fixed in 4% buffered neutral formalin, embedded in paraffin and cut at 4 μm. The pancreas is cut on three different levels (each 100 μm apart) for both the splenic and the duodenal part to obtain a representative overview. In total, there can be six measurements that are averaged. Parts of the sections are stained with hematoxylin and eosin (HE).

The other sections are immunolabeled with anti-insulin antibodies. For this purpose, sections are deparaffinized and re-hydrated, and then incubated for 25 minutes in 70% methanol and hydrogen peroxide (H₂O₂). After washing with Tris-buffer-saline (TBS, pH 7.3), the sections are incubated overnight at 4° C. with an anti-insulin antibody (Serotec, Inotech AG, Dottikon, Switzerland) in TBS+10% bovine serum, dilution 1:1000 and then for 90 minutes with an anti-guinea pig antibody (Vectastain, Vector, Reactolab SA, Servion, Switzerland) in TBS+10% bovine serum, dilution 1:200. After washing, sections are incubated with avidin-peroxidase complex (Vectastain, Vector, Reactolab SA, Servion, Switzerland) for 150 minutes and then washed again. The sections are stained with 3,3 diaminobenzidin (DAB) for 5 minutes and counter-stained with hemalum (Mayer) for 45 seconds.

On each section, the total number of islets and the relative number of pathological islets (in %) are determined. Pathological islets are defined by islet atrophy due to loss of islet cells. This is histologically recognizable as an abnormally small size and shrinkage of the islet, characterized by loss of definition of islet boundaries and displacement of exocrine tissue (single cells, acini, ducts) into the islet tissue. Evaluation is performed with a suitable microscope, such as a Nikon Eclipse E400 microscope (Nikon AG, Egg, Switzerland).

The relative number of islets (per cm² of pancreas), average islet size (in μm²), relative endocrine area (in % of the pancreatic surface), and relative β cell area (in % of the whole endocrine surface) are determined with software (program Stereo Investigator, Williston, Vt.). The different areas are assessed with the Cavalieri method. Briefly, this method allows estimation of a surface area with the help of a grid placed over that surface. The surface to be evaluated is divided in multiple squares of equal size. The surface area to be estimated is then equal to the number of intersection points of the lines of the grid which hit that surface, multiplied by the area of one square. The smaller the squares are chosen, and the higher the number of intersection points, the more accurate the estimation is and the closer to the real size of the surface.

Histological pictures are photographed, such as with a Nikon digital camera DXM 1200 (Nikon AG, Egg, Switzerland). All evaluations are preferably performed by an independent observer blinded to the treatment of the animals. Pancreas insulin content is measured after neutralization with an ELISA kit (Mercodia AB, Uppsala, Sweden).

Culture of isolated islets and MIN6 cells is performed as follows. For ex vivo studies, 6 months old male C57B¹/6J mice obtained from a commercial source, such as The Jackson Laboratories, are used. Pancreatic islets are isolated by collagenase digestion. Individual islets are handpicked and placed in RPMI 1640 culture medium (SVA, Uppsala, Sweden) containing 11 mM glucose and supplemented with 10% FBS, 2 mM L-glutamine, 60 μg/ml penicillin G, and 50 μg/ml streptomycin sulfate for an overnight recovery at 37° C. and 5% CO₂.

The next day, islets are transferred to same type of media but with 1% FBS and in the absence or presence of 0.5 mM palmitate complexed with 0.5% fatty acid free BSA (Boehringer Mannheim GmbH, Mannheim, Germany) and with or without 5, 10 or 20 μM EGCG (prepared from 20 mM stock dissolved in DMSO). Islets are exposed for 24 hours.

MIN6 cells (Bone A. J. et al., Biosci. Rep. 5:215-221, 1985), derived from mouse pancreatic β cells, are maintained in Dulbecco's Modified Eagle Medium containing 25 mM glucose and sodium pyruvate supplemented with 15% FBS, 6 mg/ml penicillin G, 5 mg/ml streptomycin sulfate (Invitrogen Inc., Carlsbad, Calif.), 2 mM L-glutamine (SVA, Uppsala, Sweden) and 50 μM β-mercaptoethanol at 37° C. and 5% CO₂. During palmitate exposure, media is supplemented with 0.5 mM palmitate and 0.5% fatty acid.

Assessment of cell viability and apoptosis is performed as follows. Cell viability is assayed with the Cytotoxicity Detection Kit (Plus Roche Diagnostics GmbH, Mannheim, Germany). The assay measures the amount of lactate dehydrogenase released from cells after lysis, which correlates inversely to the amount of live cells after treatment. Apoptosis is assayed with the cell death detection kit ELISA^{PLUS} (Roche Diagnostics GmbH, Mannheim, Germany). The ELISA measures cytoplasmic oligonucleosomes that increase after apoptosis-associated DNA degradation.

Western blot analysis is performed as follows. Samples of total protein extracted from untreated and treated islets or MIN6 cells are subjected to SDS-PAGE (15-20 μg protein per sample). After electrophoresis, proteins are transferred onto PVDF membranes. Immunoblot analyses are performed with antibodies against phosphorylated JNK 1/2, total JNK1/2 and the cleaved form of caspase 3 (all obtained from Cell Signaling Inc.). Immunoreactive bands are developed using ECL, imaged with a Gel Doc system and quantified with Quantity One software (Bio-Rad). After imaging, to verify equal protein loading, the PVDF membranes are stained with Coomassie.

### Example 18. Cytokine Production Test

This example is performed to evaluate the effect of green tea extracts, such as the mixture of different polyphenols in Phytofare® as specified above, on cytokine production. Normal human epidermal keratinocytes are treated with phorbol 12-myristate 13-acetate (PMA), which is a stimulant, and a green tea extracts. The amounts of production of three cytokines, or interleukin 1β, 6, and 8 (IL-1β, IL-6, and IL-8) are measured, thereby evaluating the anti-inflammatory effect of the green tea extracts.

Asian-derived normal human keratinocytes (NHEK, DS Pharma Biomedical Co., Ltd.) are used at the third or higher passage cultured on a serum-free medium for normal human keratinocytes (DS Pharma Biomedical Co., Ltd.).

The test sample is the green tea extract, such as Phytofare®. The cell stimulant is phorbol 12-myristate 13-acetate (PMA, Sigma-Aldrich), and the MTT reagent (Nacalai Tesque, Inc.) and SDS-HCl reagents are used for the evaluation of the survival rate. In addition, READY-SET-GO! Human Interleukin-1 β (eBioscience), READY-SET-GO! Human Interleukin-6 (eBioscience), and IL-8/NAP-1 Immunoassay Kit (Biosource) are used for the evaluation of the cytokine-producing capacity.

Cell Survival Assay (Toxicity Test). NHEK cells cultured in a flask are adjusted to 5.0×10⁴ cells/mL, and seeded on a 96 well plate in a volume of 200 μl (final concentration: 1.0×10⁴ cells/well). After culturing at 37° C. for 24 hours, 25 μl of PMA adjusted to 100 ng/mL (final concentration: 10 ng/mL) and 25 μl of the test sample adjusted to 100-1000 μg/mL (final concentration: 10-100 μg/mL) are added. After culturing for 48 hours, the culture medium is collected, and stored at −80° C. for Enzyme Linked-Immuno-Sorbent Assay (ELISA). 80 μl of new culture medium and 20 μl of MTT reagent are added to the cells, cultured for 3 to 5 hours, and 150 μl of the SDS-HCl reagent is added. After culturing for 18 to 20 hours, the absorbance at 570 nm is measured.

Measurement of the Amount of Cytokine Production by Enzyme Linked Immuno Sorbent Assay (ELISA). The culture medium stored at −80° C. is measured for the amount of cytokine by the ELISA method.

Biosource (IL-8) Pre-Coated Assay: The unfrozen culture medium is diluted three times with Standard Diluent Buffer, 50 μl of the dilution is added to a 96-well plate processed with IL-8 antibody together with a standard solution, and 50 μl of Biotin Conjugate is further added and allowed to react for 90 minutes at room temperature. After removing the solution, the well is washed with Wash Buffer four times, and then 100 μl of Streptavidin-HRP Working Solution is added and allowed to react for 30 minutes.

After removing the solution, the well is washed with Wash Buffer five times, and 100 μl of Stabilized Chromogen is added and allowed to react for 10 to 15 minutes. 100 μl of Stop Solution is added to halt the reaction, and the absorbance at 450 nm is measured. A calibration curve is prepared by plotting the absorbance of the standard solution, and the amount of IL-8 production is calculated.

eBioscience (IL-1β, 6) Non-Coated Type Assay: 100 μl of Capture Antibody diluted with Coating Buffer is added to a 96 well maxisorp plate, and cultured overnight at 4° C. After removing the solution, the well is washed with Wash Buffer five times, and then 200 μl of Assay Diluent is added and allowed to react for 1 hour at room temperature. After removing the solution, the well is washed with Wash Buffer five times, and then 100 μl of the unfrozen culture medium diluted ten and five times is added together with the standard solution, and allowed to react for 2 hours at room temperature. After removing the solution, the well is washed with Wash Buffer five times, and then 100 μl of Detection Antibody is added and allowed to react for 1 hour at room temperature. After removing the solution, the well is washed with Wash Buffer five times, and then 100 μl of Avidin-HRP is added and allowed to react for 30 minutes at room temperature.

After removing the solution, the well is washed with Wash Buffer seven times, and 100 μl of Substrate Solution is added and allowed to react for 10 to 15 minutes. 100 μl of Stop Solution is added to halt the reaction, and the absorbances at 450 and 570 nm are measured. A calibration curve is prepared by plotting the absorbance of the standard solution, and the amounts of 1 L-1β and 6 production are calculated individually.

Amount of Cytokine Production. The measured amount of cytokine production is multiplied by the dilution ratio of the cell supernatant and expressed as the amount of production (pg/mL), and then multiplied by the result of the cell survival assay and expressed as percentage taking the control as 100%, thereby allowing the comparison between equal numbers of cells. In this manner the influences of the PMA and green tea extract on the cytokine production are evaluated. If the results show that the production of IL-8 is inhibited by the addition of the green tea extract, this can indicate an anti-inflammatory effect.

### Example 19. Effect of Green Tea Extracts on Mono Mac 6 Cell Activation

The human monocytoid line, Mono Mac 6 (MM6), expresses a number of biomarkers consistent with those of resting monocytes or macrophages, and responds like human monocytes and macrophages to pro-inflammatory activating stimuli such as Phorbol Myristate Acetate (PMA). The effects of compositions containing green tea extracts and optionally, catechin standards, on MM6 cells cultured in the absence and presence of PMA are examined, to serve as models of resting and activated monocytes/macrophages. MM6 cells closely resemble a differentiated human monocyte (Ziegler-Heitbrock et al., International Journal of Cancer 41:456-461 (1988), which is hereby incorporated by reference in its entirety).

After incubation with PMA, MM6 cells secrete two so-called gelatinolytic matrix metalloproteinases, MMP-2 (gelatinase A) and MMP-9 (gelatinase B). These MMPs are also secreted by a number of tumors and by their surrounding stroma, and are implicated in inflammatory tissue injury as well as tumor invasion and metastasis.

For this example, MM6 cells (available for example from ATCC) are maintained in RPMI 1640 supplemented with 2 mM L-glutamine, 100 U/ml penicillin, 100 μg/ml streptomycin, 1 mM sodium pyruvate, 10% FCS, nonessential amino acids, 9 μg/ml insulin, and 1 mM oxalacetic acid. For assay conditions, 0.2% glucose is also added.

As a probe of cell functions, the effects of the green tea extract compositions on levels of proteinases secreted by the MM6 cell line are examined. The objective in this example is to evaluate the green tea extract compositions of the present disclosure for the capacity to diminish levels of MMPs released by activated MM6 cells.

The determination of the level of enzymes secreted by stimulated cells is performed as follows. After incubation with PMA, the levels of MMP-2 (gelatinase A) and MMP-9 (gelatinase B) in the presence of green tea extract compositions are determined by two-dimensional sodium dodecyl sulphate polyacrylamide gel electrophoresis.

To further evaluate the effects of the green tea extract compositions on MMP secretion by MM6 cells, the technique of gelatin zymography is used to examine the culture media collected as described above for the ELISA measurements. In this method, the culture media are first subjected to electrophoresis in gelatin-impregnated polyacrylamide gels in the presence of Sodium Dodecyl Sulfate (SDS-PAGE) to separate the proteins on the basis of molecular weight. The SDS is then washed out of the gels to allow at least a portion of any enzymes present to renature and the gels are incubated in a medium, which maximizes MMP activity.

MMPs dissolve the gelatin wherever they may be present. After visualizing the undigested gelatin in the bulk of the gels with a protein stain, the gels are scanned, with the MMPs appearing as clear zones against the stained background. In negative images, the MMPs appear as dark zones against a light background.

### Example 20. Autoimmune Disease Treatment

(−)-epigallocatechin-3-gallate (EGCG) modulates expression of autoantigens. Downregulation of autoantigens using the disclosed green tea extract compositions can be used to treat autoimmune diseases or symptoms associated with autoimmune diseases. A preferred mixture of different polyphenols containing EGCG is Phytofare® as specified above. Increasing expression of autoantigens can also be used to assist in the purification and isolation of autoantigens, for example to generate antibodies that can be used as diagnostics.

Serum Total Autoantibody ELISA. NOD (non-obese diabetic) mice are fed either water or water containing 0.2% Phytofare® for 3 weeks. Serum of the mice is analyzed, from the Phytofare®-water and from the water-only group. The total serum antibody levels are evaluated in each group including total ANA (against ds-DNA, ss-DNA, histones, ribonucleoproteins (RNPs), SS-A, SS-B, SM antigens, Jo-1, and Scl-70). Reduction of antibody levels in the Phytofare®-water animals will indicate that oral administration of green tea polyphenols processed as described herein can reduce the serum autoantibody levels.

Analysis of lymphocyte infiltration is performed as follows. The submandibular glands of each NOD animal are collected and the standardized scores for the inflammatory cell infiltrates are determined blindly, as described in the methods. Pathological focal scoring, using the cumulative focus score (cFS) criteria for SS (Sjogren's Syndrome) diagnosis, can be used to demonstrate potential differences in the focal areas between the groups, equivalent to differences in the total number of inflammatory cells/focus. Quantitative analysis of the areas of lymphocyte infiltration foci in H&E-stained submandibular gland sections can be used to demonstrate a difference between the groups in the number of inflammatory cells/infiltrate, with the goal of fewer cells in the salivary glands of Phytofare®/water-fed animals.

Evaluating human salivary gland acinar cell protection from TNFα-induced cytotoxicity by Phytofare®. TNFα, which is produced by inflammatory cells, is known to induce cytotoxicity in many cell types, and can be down-regulated by EGCG (Suganuma et al, 2000, Fujiki et al, 2000, Fujiki et al, 2003). Therefore, one mechanism by which EGCG could ameliorate the effects of SS could be attenuation of TNFα-cytotoxicity. This example is performed to examine the effects of Phytofare® on TNFα-induced cytotoxicity of the human salivary gland acinar cell line NS-SV-AC using the MTT assay. Phytofare® is tested for protection of NS-SV-AC cells from TNFα-induced cytotoxicity.

In the NOD mouse model of SS, oral administration of Phytofare® is tested for the effect on total serum autoantibody level and the magnitude of salivary lymphocyte infiltration.

### Example 21. HIV-1 Integrase Assay

This example is performed to measure the ability of green tea extracts as disclosed herein, including Phytofare®, on the enzyme HIV-1 integrase, which is a viral enzyme critical for productive HIV-1 infection. The green tea extracts and catechin reference standards are prepared in sterile distilled water. Reference standards EGCG (Sigma, E4143), ECG (Sigma, G3893), GCG (Sigma, G6782), CG (Sigma, C0692) and EC (Sigma, E4018) are all obtained from Sigma.

Work by others has reported that, using this method, the IC₅₀ of the pharmaceutical product Raltegravir for HIV-1 integrase was 0.26 μmol/L, and that the IC₅₀ of EGCG and CG were slightly higher than Raltegravir. However, a combination mixture of equal volumes (0.1 μmol/L) of EGCG, ECG, GCG and CG gave better inhibition than Raltegravir, and the inhibition was concentration-dependent (Jiang, F. et al., Clin. Immunol. 137:347-356, 2010). These results provide a strong rationale for testing the effect of the present green tea extracts on HIV-1 integrase activity because they contain a mixture of catechins.

The Xpress HIV-1 Integrase Assay Kit is obtained from Express Biotech International, USA, and contains sodium azide as a positive control compound that inhibits HIV-1 integrase activity. Thus, in this example the activity of the green tea extracts can be evaluated against a non-catechin control and optionally against one or more catechin reference standards.

The Xpress HIV-1 Integrase Assay Kit is used to measure the inhibitory effects of green tea extracts on HIV-1 integrase activity. Streptavidin-coated 96-well plates are coated with a couble-stranded HIV-1 LTR U5 donor substrate (DS) oligonucleotide containing an end-labeled biotin. Full-length recombinant HIV-1 integrase protein (200 nM, purified from bacteria) is loaded onto the oligo substrate.

Green tea extract, one or more catechin reference standards, or sodium azide is added to the reaction plates and then a double stranded target substrate (TS) oligo containing 3′-end modifications is added to the plate. The HRP-labeled antibody is directed against the TS 3′-end modification and the absorbance due to the HSP antibody-TMP peroxidase is measured at 450 nm. Results are expressed as percent inhibition as a function of concentration of green tea extract or catechin reference standards. Fifty percent inhibition (IC₅₀) is a standard method of comparing inhibition from different samples.

### Example 22. Effect of Green Tea Extracts on Glioblastoma Cells

This example is performed to evaluate the effect of green tea extracts disclosed herein on growth of glioblastoma cells in vitro. The green tea extracts, such as a mixture of different polyphenols in Phytofare® as specified above, are diluted in DMSO at 0.1 μl/ml and tested for their ability to induce apoptosis of U87 cells compared to olive oil/DMSO control at 48 hours as determined by TUNNEL analysis. Exponentially grown U87 cells in DMEM with 1% FBS are treated with green tea extract or olive oil at final concentration of 0.1 μl/ml, or left untreated.

After 24 hours, floating and attached cells are collected, fixed in 1 paraformaldehyde in phosphate buffered saline (PBS), washed in PBS, resuspended in 70% ethanol and analyzed by flow cytometry. The APO-BRDU kit, a two color staining method for labeling DNA breaks and total cellular DNA can be used to detect apoptotic cells. A higher number of apoptotic cells in the green tea extract-treated group compared to the control (olive oil) group indicates that the green tea extract is suitable for further testing as a treatment of glioblastoma.

The effect of the green tea extracts on pre-malignant glioblastoma can be evaluated as follows. A patient presents for treatment of a low-grade or high-grade neoplasm of the central nervous system. A green tea extract composition formulated for human use is administered to the patient over a course of treatment lasting for several weeks, with no significant side effects expected. If the patient experiences a reversal in the growth of neoplastic cells and death of existing neoplastic cells, resulting in the neoplasia becoming undetectable, this will provide further indication that the green tea compositions are useful for preventing the progression of pre-malignant glioblastoma.

The effect of the green tea extracts on glioblastoma is evaluated as follows. A patient presents for treatment of a malignant grade IV glioblastoma of the central nervous system, confirmed by manual examination and biopsy of the tumor. A green tea extract composition is administered to the patient over a course of treatment lasting for several months, with no significant side effects expected. If the patient experiences a reversal in the growth rate of tumor cells, death of existing tumor cells and reduction in tumor size, and no metastasis of the tumor, this will provide further indication that the green tea compositions are useful for reversing progression of malignant glioblastoma.

With continuing treatment, the patient is monitored for secondary symptoms of glioblastoma, long term side effects of the treatment, and metastasis of the tumor, and if these markers are all absent, or reduced compared to a control, the usefulness of the green tea compositions in glioblastoma therapy is further indicated.

### Example 23. Effect of Green Tea Extracts on Prostate Cancer Cells

This example is performed to evaluate the effect of green tea extracts disclosed herein on prostate cancer cells in vitro. A preferred mixture of different polyphenols is Phytofare® as specified above. A series of dilutions of the green tea extracts in DMSO are prepared, and the dilutions are added to LNCaP growth medium so that all doses tested have equivalent (0.1%) DMSO levels.

Cell growth curves are prepared by counting cells at 24, 48, and 72 hours, and are compared to control cells treated at the same times with 0.1% DMSO alone. Apoptosis in these cultures is evaluated by Western blot analysis of PARP cleavage and measurement of caspase-3 activity using a calorimetric substrate assay. Effects on purified COX-2 enzyme activity is measured using a calorimetric assay, and effects on COX-2 protein expression is determined via Western blot analysis of protein extracts from treated cells.

If LNCaP cell growth is suppressed by the inventive compositions, such as a 50% or 75% or more reduction in cell number in treated cultures compared to controls, the green tea extracts are suitable for further investigation in prostate cancer treatment. Evidence of PARP cleavage fragments and upregulated caspase-3 activity correlate with an apoptotic effect that is not expected to be found in controls. COX-2 activity may also be decreased in the presence of the green tea extracts.

The effect of the green tea extracts on pre-malignant prostate neoplasia can be evaluated as follows. A patient presents for treatment of a pre-malignant neoplasia of the prostate. A green tea extract composition is administered to the patient over a course of treatment lasting for several weeks, with no significant side effects expected. If the patient experiences a reversal in the growth of neoplastic cells and death of existing neoplastic cells, resulting in the neoplasia becoming undetectable, this will provide further indication that the green tea compositions are useful for preventing the progression of pre-malignant neoplasia of the prostate. The effect of the green tea extracts on prostate epithelioma can be evaluated as follows.

A patient presents for treatment of a malignant stage B epithelioma of the prostate, confirmed by elevated PSA test, manual examination, and biopsy of the tumor. A green tea extract composition is administered to the patient over a course of treatment lasting for several months, resulting in no significant side effects. If the patient experiences a reversal in the growth rate of tumor cells, death of existing tumor cells and reduction in tumor size, and no metastasis of the tumor, this will provide further indication that the green tea compositions are useful for reversing progression of neoplasia of the prostate.

With continuing treatment, the patient is monitored for secondary symptoms of neoplasia of the prostate, long term side effects of the treatment, and metastasis of the tumor, and if these markers are all absent, or reduced compared to a control, the usefulness of the green tea compositions in therapy of neoplasia of the prostate is further indicated.

### Example 24. Effects of Green Tea Extracts on Growth of MDA-MB-435 and MCF-7 Breast Cancer Cells

In this example, the effect of green tea extracts of the disclosure and optionally at least one catechin standard such as EGCG on the proliferation and growth of MDA-MB-435 estrogen receptor-negative human breast cells is studied in vitro, as measured by the incorporation of [³H] Thymidine.

The green tea extracts and catechin standards are prepared as discussed above. Tissue culture medium and fetal calf serum are obtained from a commercial supplier, such as Gibco, Burlington, ON. [³H] Thymidine is obtained from a commercial supplier, such as from ICN, Irvine, Calif.

MDA-MB-435 cells (human breast cancer cells) are maintained at 37° C. in a minimum essential medium, supplemented with 10% (v/v) fetal bovine serum. The medium is equilibrated with a humidified atmosphere of 5% CO₂. Stock cultures are seeded at a density of 2×10⁴ cells/ml and allowed to multiply for 48 to 72 hours.

Incorporation of [³H] Thymidine into DNA is measured as follows. MDA-MB-435 cells are plated at 2×10⁴ cells/well in 96-well, flat bottomed, culture plates in a total volume of 200 μl of medium and incubated at 37° C. for 48 hours with or without test compounds. [³H] Thymidine (0.5 μCi/well) is then added and after 4 hours the cells are harvested onto a glass fibre filter paper using a semiautomatic 12-well cell harvester (for example, from Skatron Inc., Sterling, Va.). Radioactivity on the filter paper is counted using Scinteverse in a liquid scintillation counter.

For the following growth experiment, MDA-MB-435 cells are plated at 1×10⁴ cells/dish in 60 mm dishes with or without green tea extracts or catechin standards. The cells are removed by trypsinization at specified times and counted using a hemocytometer.

The viability of cells is measured by MTT assay (for example, Hansen, M. B. et al., J. Immunol. Meth., 119, 203-210, 1989). In this assay a tetrazolium salt, 3-[4,5-dimethylthiazole]-2,5-diphenyltetrazolium bromide, MTT, is reduced to a blue formazan product by mitochondrial dehydrogenases that are active in living cells. The intensity of the blue color developed is a measure of cell viability.

MDA-MB-435 cells (8×10⁴/well) are seeded in 96-well, flat-bottomed tissue culture plates with various concentrations of the test compounds, in a total volume of 200 μl/well of medium. Forty-eighty hours later, MTT (25 μl of 5 mg/ml) is added to each well. After 3 hours, 100 μl of extraction buffer, consisting of 20% SDS dissolved in a 50% DMF, 50% water solution at pH 4.0, is added. The blue color formed is measured at 570 nm in a Dynatech MRX Microplate Reader. The percentage of cells surviving is determined by comparing the absorbance of the treated cells with that of the control.

The effect of green tea extracts and optionally catechin standards on MCF-7 cells is measured as follows. Tissue culture medium, fetal calf serum and fingizone are obtained from a commercial supplier, such as Gibco, Burlington, ON. Fetal calf serum treated with dextran-coated charcoal (FCS/DCC) is obtained from a commercial supplier such as Cocalico Biologicals Inc., Reamstown, Pa. [³H] Thymidine is obtained from obtained from a commercial supplier such as ICN, Irvine, Calif. MTT and all other chemicals are obtained from obtained from a commercial supplier such as Sigma.

For cell culture, MCF-7 cells (estrogen receptor-positive human breast cancer cells) are maintained at 37° C. in minimum essential medium containing 3.7 g of sodium bicarbonate per liter supplemented with 10% v/v fetal calf serum and 1% (v/v) fungizone (antibiotic/antimycotic), supplemented with 1 mM sodium pyruvate and 10 μg/mL insulin, in humidified atmosphere of 5% carbon dioxide. Stock cultures are seeded at a density of 2×10 cells/mL and passaged weekly, using 0.25% trypsin.

Incorporation of [³H] Thymidine. For the MCF-7 cells, the growth medium is exchanged for pheno red-free medium containing 10% fetal calf serum that has been treated with dextran-coated charcoal (FCS/DCC) five days prior to use. The cells are then trypsinized and 2×10⁴ cells/well are plated as described above.

After 2 days the medium is removed and the cells are incubated for 5 days in an experimental medium containing 2.5% FCS/DCC with or without the green tea extracts or catechin standards. [³H] Thymidine (0.5 μCi/well) is then added and the cells are harvested as described above. The concentration at which 50% inhibition occurred is determined by comparing the number of disintegrations per minute for the treated cells with that obtained for the control cells.

For a growth experiment, MDA-MB-435 and MCF-7 cells are plated at 1×10⁴ cells/dish in 60 mm dishes with or without green tea extract or catechin standards at their IC₅₀ concentration in a total volume of 7 mL. The cells are removed by trypsinization at the specified times and counted, using a hemocytometer.

### Example 25. Effect of Green Tea Extracts on Breast Cancer Cell Lines

Green tea extracts, and optionally one or more catechin standards such as EGCG, are screened for their cytotoxicity on estrogen receptor (ER)-positive (MCF-7) or ER-negative (MDA-MB-231) human breast cancer cells. The mechanism of anti-proliferative activity of the green tea extracts can also be tested using an in vitro aromatase enzyme assay and Western blot with anti-caspase-7.

### Example 26. Effect of Green Tea Extracts on Cancer Cell Lines

This example is performed to determine the effects of green tea extracts on A549 tumor growth in nude mice, and angiogenesis. Human non-small-cell lung carcinoma cell line A549 and cervical carcinoma cell line HeLa are maintained in Dulbecco's Modified Eagles's Medium (DMEM, Sigma Aldrich), and retinoblastoma cell line Y59 and human leukemia cell line U937 are maintained in RPMI 1640 supplemented with 10% fetal bovine serum (FBS, Gibco) and antibiotic-antimycotic solution (Wako Pure Chemicals, Osaka, Japan) at 37° C., 5% CO₂. All cell lines are obtained from ATCC, Rockville, Md.

The effects of green tea extracts on cell growth and viability are measured as follows. Cells (1×10⁵ cells per well for 35 mm plate) are seeded onto plates, cultured overnight, and treated with different concentrations of green tea extract and optionally with green tea catechin standards including EGCG. Concentrations tested range from 0 μM to 50 μM and 100 μM catechins. The viability of cells is determined by counting the numbers of living and dead cells by trypan blue exclusion method. Viability is tested at different intervals, such as 24 hours, 48 hours, and 72 hours after addition of the green tea extract or catechin standard.

The effects of green tea extracts on endostatin and VEGF concentrations is measured by ELISA. A549 cells are cultured in the presence and absence of green tea extracts and catechin standards at the concentrations used for assaying cell growth and viability, for various times such as 0, 3 and 6 hours. The supernatants of the cell cultures are harvested and stored at −20° C. until use. The levels of endostatin and VEGF in the culture media are analyzed by enzyme immunoassay (ELISA, Cytoimmune, College Park, Md.; genzyme Techne, Minneapolis, Minn.) following the manufacturers' instructions.

The effects of green tea extracts on tumor growth in vivo is measured as follows. BALB/c nude (nu/nu) male mice are obtained from a commercial source, such as The Jackson Laboratories, Bar Harbor, Me. A549 cells (1×10⁷ cells in 0.1 ml of PBS) are inoculated subcutaneously into the right side of the backs of mice, using for example three mice per treatment group. Tumor growth is monitored by caliper measurement every 2 to 3 days and tumor size is calculated by a standard means, such as the formula 1×w²/[length (l) and width (w)].

Three days after inoculation, drinking water bottles are replaced with bottles containing green tea extracts solutions, catechin standard solutions, or control solutions. An exemplary control solution is an 0.05% DMSO solution for use if the catechin standard, such as EGCG, is dissolved in DMSO. The green tea extract-containing solution is compared with the same solution provided but without the green tea extract. On the final day of treatment, the mice are sacrificed, and the tumors removed, fixed in formalin, and paraffin embedded.

### Example 27. Effect of Green Tea Extracts on Esophageal Cancer Cell Lines

Human esophageal cancer cell lines SKGT-4 and TE-8 as previously described (Xu, X. C. et al., Cancer Res. 59:2477-2483, 1999; Li, M. et al., Cancer Epidemiol. Biomarkers Prev. 9:545-549, 2000) are grown in Dulbecco's modified Eagle's minimal essential medium (DMEM), supplemented with 10% fetal bovine serum (FCS), at 37° C. in a humidified atmosphere of 95% air and 5% CO₂. For extract or catechin treatment, these cells are grown in monolayer overnight and then treated with or without green tea extracts, one or more catechin standards such (−)-epigallocatechin-3-gallate (EGCG), optionally curcumin, and combinations of these agents for up to 5 days. The agents are dissolved in dimethyl sulfoxide (DMSO) and then diluted before use.

The concentration of curcumin is 20 or 40 μmol/L and EGCG is 20 or 40 μmol/L (obtained from commercial sources, such as LKT Laboratories, Inc., St. Paul, Minn., USA). The concentrations for the agent combinations are the same as those used individually. For the methyl thiazolyl tetrazolium (MTT) assay, 20 μl of MTT (5 mg/mL, Sigma, St Louis, Mo., USA) is added to each well of the 96-well plates and incubated for an additional 4 h. After the growth medium is removed, 100 μl of DMSO is added to the wells to dissolve the MTT crystal, and the optical densities are measured with an automated spectrophotometric plate reader at a single wavelength of 540 nm. The percentage of cell growth is calculated using the formula: % control=ODt/ODc×100, where ODt and ODc are the optical densities for treated and control cells, respectively. The data are then optionally analyzed statistically using the Student's t test.

For the tumor cell invasion assay, Boyden chambers coated with Matrigel are obtained from BD Biosciences (Bedford, Mass., USA) for assaying tumor cell invasion ability. Esophageal cancer cells SKGT-4 and TE-8 are first starved in medium without FCS overnight, and the cells (5×10⁴) are resuspended in the FCS-free medium and placed in the top chambers in triplicate. The medium in the top chambers contains green tea extracts, and optionally curcumin (40 μmol/L), and/or EGCG (40 μmol/L), or their combinations.

The lower chamber is filled with DMEM and 10% FCS as the chemoattractant and incubated for 48 h. The upper surface is then wiped with a cotton swab to remove the remaining cells. The cells which invaded the Matrigel and attach to the lower surface of the filter are fixed and stained with 1% crystal violet solution. The cells in the reverse side are photographed (5 microscopic fields at 100× magnification per chamber). The cells in the photographs are then counted, and the data are summarized as mean±SD and presented as a percentage of the controls (mean±SD). The data are then optionally analyzed statistically using the Student's t test.

For protein extraction and Western blotting, the cells are grown and treated with or without the agents for 2 days. After that, total cellular protein is extracted using a standard method, such as described in Li, M. et al., Cancer Epidemiol. Biomarkers Prev. 9:545-549 (2000). Samples containing 50 μg of protein from each treatment are then separated by 10%-14% on sodium dodecyl sulfate-polyacrylamide gel electrophoresis gels and transferred electrophoretically to a Hybond-C nitrocellulose membrane (obtained commercially, such as from GE-Healthcare, Arlington Heights, Ill., USA) at 500 mA for 2 h at 4° C.

The membrane is subsequently stained with 0.5% Ponceau S containing 1% acetic acid to confirm that the proteins are loaded equally and to verify transfer efficiency. Next, the membranes are subjected to Western blotting by overnight incubation in a blocking solution containing 5% bovine skimmed milk and 0.1% Tween 20 in phosphate-buffered saline (PBS) at 4° C.

The next day, the membranes are first incubated with primary antibodies and then with horse anti-mouse or goat anti-rabbit secondary antibodies (GE Healthcare) for enhanced chemiluminescence detection of antibody signals. The antibodies used are anti-Ki67 (Vector Laboratories, Burlingame, Calif., USA), anti-phosphorylated Erk1/2 (Cell Signaling Technology, Danvers, Mass., USA), anti-COX-2 (BD Transduction Laboratories, Lexington, Ky., USA), and anti-6-actin (Sigma-Aldrich, St. Louis, Mo., USA).

For animal experiments, esophageal cancer SKGT-4 cells are grown and treated with or without these agents for 3 days before injection (the doses are the same as above). Nu/nu nude mice (6-8 wk of age) are treated with or without green tea extract and optionally curcumin (50 μg/kg per day), and/or EGCG (50 μg/kg per day), and their combinations (the same doses used individually) orally for two days and then subcutaneously injected in the right flank through a 22-gauge needle with 2×10⁶ tumor cells mixed with 50% Matrigel (BD Biosciences) for a total volume of 200 μl per mouse.

The animals are then continuously treated with or without these drugs orally five days per week for an additional thirty days and monitored for tumor formation and growth daily. The tumor mass volumes, measured weekly with a vernier caliper, are calculated as follows: length×width²/2. At the end of the experiments, the tumor xenografts are taken excised, weighed and the results compared and summarized to determine the effect of green tea extract treatment on tumor growth compared to controls and to catechin standards such as EGCG, optionally with or without curcumin.

### Example 28. Effect of Green Tea Extracts on Breast and Colon Cancer Cell Lines

In this example, 5×10⁴ breast cancer cells (MDA MB 231) are seeded in each of the wells of 24-well plate. Control group refers to breast cancer cells that are grown in Liebovitz's media supplemented with 10% fetal bovine serum (FBS). Treatment group refers to breast cancer cells that are grown in Liebovitz's media supplemented with 10% fetal bovine serum (FBS) plus green tea extracts, or with a catechin standard, such as of one or more of 0, 10, 20, 50, 100 and 200 mg/ml of EGCG. Plates are incubated in ambient air (without supplemental CO²) for four days.

At the end of the incubation, the culture media are withdrawn and the cells in each well are stained with MTT. Excess MTT stain is washed off. The MTT-stained cancer cells are dissolved in 1 ml DMSO solution. The optical density (OD) of the solution is determined for each well. The OD for the well is directly proportional to the number of dead cells. The OD of the MTT stained cancer cells that are previously cultured in the absence of EGCG is used as a reference and is considered as 100. Percent inhibition is calculated by using the formula: % Inhibition=(OD of Reference−OD of the Test Treatment)/OD of Reference×100%.

The effects of green tea extracts on proliferation of human colon cancer cells (HCT116) are measured using the same methods and treatments as for the MDA MB 231 cells, except that the HCT116 cells are grown in McCoy's 5A medium with 10% fetal bovine serum in 5% CO² atmosphere.

### Example 29. Melanin Production Inhibition Test

Human melanoma cells are treated with green tea extract, and the amount of melanin production is compared with that of a negative control, thereby studying the production inhibitory effect (whitening effect). Human melanoma cells (HMVII) at the third or higher passage are cultured in a culture solution of a Ham F-12 culture medium (manufactured by Nissui Pharmaceutical Co., Ltd.) containing 10% FBS, mixed with 0.5% penicillin-streptomycin (manufactured by Gibco). The test samples are green tea extracts as described above.

Melanin Production Assay. 960 μl of human cells HMVII cultured in Ham F-12 culture medium containing 10% FBS is seeded on a 24-well plate containing 10% FBS to give a final concentration of 1.0×10⁵ cells/well (seeding concentration: 1.04×10⁵ cells/nil), and cultured for 24 hours in a CO₂ incubator. 20 μl of melanin production hormone (α-MSH) adjusted to a final concentration of 100 ng/ml (addition concentration: 5.0 μg/ml) and 20 μl of test sample adjusted to 10 μg/ml (500 μg/ml) or 100 μg/ml (5000 μg/ml) are added, and cultured for 72 hours.

The culture medium is removed, washed with PBS, and then 100 μl of a trypsin EDTA solution is added thereby removing the cells from the plate, and 900 μl of 1N sodium hydroxide aqueous solution is added. The cells are allowed to stand at room temperature for 20 to 24 hours thereby causing cytolysis, and then the melanin content is measured at an absorbance of 475 nm.

Cell Survival Rate Assay. In order to correct the influence of the green tea extract on the cell survival rate (proliferation rate), the survival rate is calculated by the MTT method using the control as a standard, and the measured melanin value is multiplied by the survival rate. 160 μl of mouse cells B16F1 is seeded on a 96-well plate at 2.0×10⁴ cells/well (1.25×10⁵ cells/nil), and cultured for 24 hours. 20 μl of melanin production hormone (α-MSH) adjusted to a final concentration of 100 ng/ml (1.0 μg/mL) and 20 μl of test sample adjusted to 10 μg/ml (100 μg/ml) or 100 μg/ml (1000 μg/ml) were added, and cultured for 72 hours. The old culture medium is removed, 80 μl of new culture medium and 20 μl of MTT reagent were added, and cultured for 3 to 5 hours. 150 μl of SDS-HCl reagent is further added, cultured for 18 to 20 hours, and then the absorbance at 570 nm is measured.

In the MTT test, if addition of green tea extract to the HMVII cells generally increases the survival rate, this indicates that the green tea extract has cell growth-promoting activity. Therefore, even when the measured amount of melanin production of the sample is higher than that of the control, the sample value became lower than the control value after correction with the survival rate, indicating that the sample has melanin production inhibitory activity.

### Example 30. Inhibitory Effects of Green Tea Extracts on Invasion of Matrigel by Cancer Cells

The Matrigel invasion assays are conducted using Matrigel (Becton Dickinson) inserts in compatible 24 well plates. Human fibroblast cells are seeded and grown in the 24-well plates using culture media containing ˜10% serum. When the fibroblasts reach coalescence, the culture media with serum is withdrawn and replaced with fresh media without serum. A combination of green tea extract plus dietary composition, or catechol standard such as EGCG plus dietary composition, are added to the media without serum and human cancer cells are seeded on the upper surface of the Matrigel inserts.

After 18 hours, the media are withdrawn. Some media are saved for zymogram studies. The cells on the upper surface of the inserts are gently scrubbed away with cotton swabs. The cells that have penetrated the Matrigel membrane and have migrated into the lower surface of the Matrigel are stained with Quick Stain and are counted under a microscope.

Zymogram studies. The media (25-30 μl) from the Matrigel invasion studies is applied to Novex zymogram gels (Invitrogen). The gel plates are developed and stained as recommended by the manufacturer. The matrix-metalloproteinases (MMPs) bands are identified on the basis of their known molecular weights.

Morphological studies. The morphology of human cancer cells that had migrated into the lower surface of the Matrigel membrane are stained with Quick Stain and are photographed under a microscope (such as at 100×). The general procedure for Matrigel invasion assay has been described above. In this assay, human breast cancer cells (5×10⁴) are seeded on each insert. Green tea extracts or catechol standard such as EGCG are added to Leibovitz's media. The plates are incubated in an incubator in ambient air without supplemental CO₂.

In a previous study (Netke, U.S. Pat. No. 6,939,860), a composition comprising 20 or 50 μg/ml EGCG in the media inhibited invasion by breast cancer cells by about 26% and 100% respectively. Using EGCG as a standard the inhibition of invasion by breast cancer cells treated with green tea extracts of this disclosure is evaluated and compared.

Effects of green tea extracts on invasion through Matrigel by human melanoma cells (A2058). The general procedure for Matrigel invasion assay is described above. Human melanoma cells (A2058) (5×10⁴) are seeded on each insert. Green tea extracts or catechol standard such as EGCG are added to DMEM. The plates are incubated in an incubator under 5% CO₂ atmosphere.

In a previous study (Netke, U.S. Pat. No. 6,939,860), a combination of ascorbic acid (100 μM)+proline (140 μM)+lysine (400 μM) caused only 13% inhibition, whereas a combination of ascorbic acid (100 μM)+proline (140 μM)+lysine (400 μM) plus 20 μg/ml EGCG completely prevented the invasion of melanoma cells through the Matrigel. Using EGCG as a standard in this Example, the inhibition of invasion by melanoma cancer cells treated with green tea extracts of this disclosure is evaluated and compared.

To study the effects of green tea extracts on MMP2 Production by human breast cancer cells (MDA MB 231), the media from various treatments in the Matrigel invasion assay (described above) are applied to Novex Zymogram Gel (Invitrogen). The plates are developed and stained as recommended by the manufacturer. The matrix metalloproteinases (MMPs) bands are identified on the basis of their known molecular weights.

In a previous study (Netke, U.S. Pat. No. 6,939,860), Zymogram of the culture media from the Matrigel invasion assays indicated that 20 ug/ml EGCG in the media reduced the production of MMP2 and completely inhibited the production of MMP9. At concentrations of 50 μg/ml and 100 ug/ml of EGCG, the activities of both MMP2 and MMP9 were completely inhibited in that study. Using EGCG as a standard in this Example, the inhibition of MMP production by breast cancer cells treated with green tea extracts of this disclosure is evaluated and compared.

The effects of green tea extract on the cell morphology of human melanoma cells (A2058) is studied by reviewing the micrographs of the cancer cells in basal media as they migrate through the Matrigel.

In a previous study (Netke, U.S. Pat. No. 6,939,860), inclusion of the combination of ascorbic acid (100 μM)+proline (140 μM)+lysine (400 μM) in the media altered the morphology of the cells. The distension of the cells with distinct enlargement of nucleus was evident. Addition of 20 ug/ml of EGCG to the combination of ascorbic acid (100 μM)+proline (140 μM)+lysine (400 μM) in the media caused extensive apoptotic changes. Using EGCG as a standard in this Example, the effect of green tea extract on the morphology of cancer cells treated with green tea extracts of this disclosure, including evaluation of apoptosis, is evaluated and compared.

### Example 31. Effect of Green Tea Extracts on Breast Tumor Angiogenesis and Growth

This example relates to measuring the effect of green tea extracts of the disclosure on on breast tumor angiogenesis and growth by assaying the activation of HIF-1α and NFκB, and VEGF expression

EGCG for optional use as a catechin standard is obtained from a commercial source such as Sigma Chemical Co. (St. Louis, Mo.). Human estrogen-receptor positive breast cancer (MCF-7) cells and human triple negative breast cancer (MDA-MB-231) cells are obtained from a commercial source such as from the American Type Culture Collection (ATCC, Rockville, Md.). All breast cancer cells are maintained as monolayer cultures in RPMI Medium 1640 (GIBCO) supplemented with 10% FBS (HyClone), 100 U/ml penicillin, 100 μg/ml streptomycin, and 0.25 μg/ml amphotericin B, and incubated at 37° C. in a humidified 5% CO₂/air injected atmosphere.

Female C57BL/6 mice at 7 weeks of age are obtained from The Jackson Laboratory (Bar Harbor, Me.). Twenty four is a suitable number for this experiment. The mice are allowed to acclimate for 1 week with standard chaw diet (for example, Teklad, Harlan Sprague Dawley; Indianapolis, Ind.) and tap water before beginning the experiments. The eight week old female mice (n=16) are inoculated with 10⁶ cells suspended in 100 μl of phosphate-buffered saline into the left fourth mammary gland fat pad. Then, 8 mice receive green tea extract of the disclosure, 8 mice receive EGCG (25 mg/50 ml) in drinking water for 4 weeks, and 8 control mice receive drinking water only. Each mouse (20 g) is expected to drink about 2 to 4 ml of water per day. The EGCG amount ingested is around 50 to 100 mg/kg/day.

The body weight of the mice is monitored weekly. Tumor size is monitored every other day in two perpendicular dimensions parallel with the surface of the mice using dial calipers. At the end of the experiment, blood samples, tumors, heart and limb muscles are collected for measuring VEGF expression using ELISA and average microvascular density (AMVD) or capillary density (CD) using CD31 immunohistochemistry.

The quantification of blood vessels in mouse breast tumor, the heart and limb muscle is determined with the modification of a previously reported method (Young, E. et al., Cancer Biol Ther 10:703-711, 2010; Gu, J. W. et al., Microcirculation 11:689-697, 2004). Briefly, the tissues are fixed in 4% neutrally buffered paraformaldehyde. For the heart left ventricular and limb muscle samples, consecutive thin transverse cryosections (5 μm) are cut along the base-apex axis.

Consecutive thin cryosections (5 μm) of OCT compound (Sakura Finetek, Torrance, Calif.) embedded tissue samples are fixed in acetone at 4° C. for 10 min. After washing in phosphate-buffered saline (PBS), the sections are treated with 3% H₂O₂ for 10 minutes to block endogenous peroxidase activity and are blocked with normal rabbit serum. Then, the sections are washed in PBS and incubated with rat anti-mouse CD31 (PECAM-1) monoclonal antibody (BD Pharmingen, San Diego, Calif.) at a 1:200 dilution overnight at 4° C. Negative controls are incubated with the rat serum IgG at the same dilution.

All sections are washed in PBS containing 0.05% Tween-20, and are then incubated with a second antibody, mouse anti-rat IgG (Vector laboratories, Burlingame, Calif.) at a 1:200 dilution for 1 hour at room temperature again followed by washing with PBS containing 0.05% Tween-20. The sections are incubated in a 1:400 dilution of Extravadin Peroxidase (Sigma, St. Louis, Mo.) for 30 min. After washing in PBS containing 0.05% Tween-20, the sections are incubated in peroxidase substrate (Vector laboratories, Burlingame, Calif.) for 5 min. The sections are washed in PBS containing 0.05% Tween-20 and are counterstained with hematoxylin. A positive reaction is indicated by a brown staining.

The microvascular vessels are quantified by manual counting under light microscopy. A microscopic field (0.7884 mm²) is defined by a grid laced in the eye-piece. At least 20 microscopic fields are randomly acquired from each tumor for analysis. Any endothelial cell or cell cluster showing antibody staining and clearly separated from an adjacent cluster is considered to be a single, countable microvessel. The value of average microvascular density (AMVD) or capillary density (CD) is determined by calculating the mean of the vascular counts per mm² obtained in the microscopic fields for each tissue sample.

Protein levels of VEGF in plasma, breast tumor, the heart, the limb muscle, and the medium cultured with cells are determined using mouse VEGF ELISA kits (R&D Systems, Minneapolis, Minn.), according to the manufacturer's instructions. The total proteins of breast tumor, the heart, the limb muscle, and cultured cells are extracted using NE-PER Cytoplasmic Extraction Reagents (Pierce, Rockford, Ill.), according to the manufacturer's protocol. The total protein concentration of these tissue extractions is determined using a Bio-Rad Protein Assay (Bio-Rad Laboratories, Hercules, Calif.). The protein concentrations of VEGF are normalized and expressed as pictograms per milligram of total tissue or cell extraction protein.

To assay to proliferation of cultured breast cancer cells, the MCF-7 and MDA-MB-231 cells are seeded into 6-well tissue culture plates using RPMI Medium 1640 (GIBCO) supplemented with 10% FBS (HyClone), 100 U/ml penicillin, 100 μg/ml streptomycin, and 0.25 μg/ml amphotericin B, and incubated at 37° C. in a humidified 5% CO₂/air injected atmosphere. When the monolayer reach about 80% confluence, the cells are washed with PBS and incubated with fresh RPMI Medium 1640 with 10% FBS in the absence and presence of EGCG (0, 10, 50 μg/ml) for 18 hours. ³H-thymidine incorporation assay is used to determine the cell proliferation during the last 6 hours of incubation as previously described (Gu, J. W. et al., Cancer 103:422-431, 2005).

The migration assay is performed as follows. Migration is determined using BD BioCoat Matrigel Invasion Chamber (BD Bioscience Discovery Labware, Sedford, Mass.) according to a previous study, in which only invasive cells digested the matrix and moved through the insert membrane (Gu, J. W. et al., Cancer Biol. Ther. 8:514-521, 2009.) 1×10⁵ E0771 cells per well in 0.5 ml medium (RPMI Medium 1640) are seeded in the matrigel-coated upper compartment (insert) of a Transwell (24-well format, 8-μm pore) in the absence of and presence of green tea extract, EGCG (0, 10, 20, 50 μg/ml), and one or more other catechin standards. The medium with 10% FBS is added to the lower part of the well.

After overnight incubation at 37° C. and 5% CO₂, cells on the upper surface of the insert are removed using a cotton wool swab. Migrated cells on the lower surface of the insert are stained using DiffQuit (Dada Behring, Düdinen, Switzerland). The images of migrated cells are taken and the number of migrated cells is counted using a microscope (Leica, Germany) in a 20× objective.

HIF-1α and NFκB activation (motif binding) assays are performed as follows. HIF-1α and NFκB activation is measured in cultured cells in the absence and presence of green tea extracts and optionally in the absence and presence of one or more catechin standards such as EGCG (0 and 50 mg/ml), to investigate whether the down-regulation of VEGF by EGCG is associated with the inhibition of HIF-1α and NFκB activation (n=6). The nuclear proteins are extracted by using Active Motif (Carlsbad, Calif.) nuclear extract kit. 20 μg nuclear proteins from each sample is used in the TransAM HIF-1α or NFκB p65 kit (Active Motif), which can measure the binding of activated HIF-1α or NFκB to its consensus sequence attached to a microwell plate, according the manufacturer's instructions.

Preferably, all determinations are performed in duplicated sets. Data can be presented as mean±SE. Statistically significant differences in mean values between the two groups are tested by an unpaired Student's t-test. Linear regression is performed by the correlation analysis between two continuous variables. A value of P<0.05 is considered statistically significant. All statistical calculations are performed using SPSS software (SPSS Inc., Chicago, Ill.).

### Example 32. Neuroprotective Effect of Green Tea Extracts

The present example compares the neuroprotective effect of EGCG with green tea extracts of the disclosure, when administered after the induction of cell damage. This is also referred to as “neurorescue”. As a model of a progressive mode of death, PC12 cells are subjected to serum-starvation conditions for a period of 1 or 3 days before administration of EGCG (0.1-10 μm) or green tea extract for up to 3 days.

In a previous study, in spite of the high percentage of cell death, single or repetitive administration of EGCG (1 μm) significantly attenuated cell death (Reznichenko, L. et al., Journal of Neurochemistry 93:1157-1167, 2005.) In that study, the neurorescue effect of EGCG was abolished by pre-treatment with the protein kinase C inhibitor GF109203X (2.5 μm), suggesting the involvement of the protein kinase C pathway in neurorescue by the drug. This is consistent with the rapid (15 min) translocation of the protein kinase C alpha isoform to the cell membrane in response to EGCG. The correlative neurite outgrowth activity of EGCG on PC12 cells may also contribute to its neurorescue effect.

The present example compares green tea extract with the known effect of EGCG, and results of equal or better protection will indicate that the green tea extracts disclosed herein can have a positive impact on aging and neurodegenerative diseases to retard or perhaps even reverse the accelerated rate of neuronal degeneration.

### Example 33. In Vitro Digestion and Uptake by Caco-2 Cells

This example is performed to assess the in vitro digestion of homogenized green tea extracts of the disclosure and the subsequent uptake by Caco-2 cells, which are a model for mature enterocytes. In vitro digestion is performed on green tea extracts of the disclosure and optionally comparative samples of commercially available green tea products and unprocessed green tea leaves. The assays are optionally performed in triplicate to determine the percent digestive stability and the percent micellerization according to the protocol of Thakkar et al. (J. Nutr. 137:2229-2233, 2007), without the “oral phase.”

This process uses a “gastric phase” where the pH of the green tea homogenate is adjusted to 2.5±0.1, pepsin is added at 40 mg/ml, and the mixture is incubated in a shaking water bath at 37° C. for 1 hour. In the subsequent “intestinal phase” the pH is adjusted to 6.5±0.1, porcine pancreatic lipase, pancreatin, and bile extract are added and the mixture is incubated in a shaking water bath at 37° C. for 2 hours. The micelle fraction is then isolated from the digesta by centrifugation at 5000×g for 45 min at 4° C. and filtration (0.22 mm pore size) of the collected aqueous (micelle) fraction (Thakkar S K, et al., J. Nutr. 137:2229-2233, 2007).

The aqueous fractions are then applied to Caco-2 cells. Stock cultures of Caco-2 (HTB-39) cells are obtained from American Type Culture Collection and are maintained as described (Chitchumroonchokchai C, et al., J. Nutr. 2004; 134:2280-2286). The Caco-2 human cell line exhibits characteristics of mature enterocytes (Ellwood K C, et al. Proc. Soc. Exp. Biol. Med. 202:440-446, 1993). T75 flasks of Caco-2 cells are grown 10-14 days post confluency.

Following in vitro digestion, the aqueous fractions containing the micelles are collected and each diluted 1:4 with Dulbecco's minimum essential medium (DMEM) and 12.5 mL of the medium is added to each flask. At the end of 4 hours the medium is collected and cells are washed with ice cold phosphate-buffered saline (PBS) with albumin, which is also collected and combined with the medium. The cells are washed twice with ice cold PBS and the wash is discarded.

10 ml of ice cold PBS is then added to each flask, the cells are scraped, collected, the process repeated. The collected cells in PBS are centrifuged at 2000×g at 4° C. for 5 min and the supernatant is discarded. The cell pellet is resuspended in 2 mL PBS and extracted for HPLC analysis. Resuspended cell pellets (100 μl) are used for a protein assay using to a standard method, such as Bradford (Bradford M. M., Anal. Biochem. 72:248-254, 1976). Aliquots of the whole digestion, aqueous (micelle) fraction, fresh 1:4 media, spent media, and the cells are extracted with tetrahydrofuran (THF) and hexane. Briefly, 2 mL of THF is added to 2 mL of sample and vortexed, then 3 ml of hexane is added and vortexed, and centrifuged at 5000×g for 5 min to separate phases. The upper layer is collected and the extraction is repeated two times.

Extracts are dried under a stream of nitrogen and redisolved in 2:1 isopropanol/dichloromethane and filtered thru a 0.22 micron syringe filter and injected into the HPLC. HPLC analysis is performed with a Waters 1525μ Binary HPLC Pump with a Waters 996 Photodiode Array Detector and a Waters 717plus Autosampler set at 10° C. A YMC Carotenoid 5 μm particle (4.6×150 mm) Column with a YMC Carotenoid 5 μm particle (4.0×20 mm) Guard Cartridge is used. Separation is achieved by gradient elution with a binary mobile phase of methanol-0.1% (v/v) formic acid (FA) as Solvent A (80:20) and MTBE-methanol-0.1% FA as Solvent B (78:20:2) at a flow rate of 1.8 mL/min. Initial conditions are held at 100% A for 1 min then a linear gradient to 40:60 A:B over 5 min, followed by a linear gradient to 100% B over 9 min, a linear gradient back to 100% A for 1 min, and held at 100% A for 4 min for a final chromatographic run time of 20 min. Identification and quantification of the catechin compounds of interest is accomplished by comparison with synthetic standards run in a dilution series before and after the samples.

The results of this example provide information about the bioaccessibility/bioavailability of catechins from green tea extracts of the disclosure in comparison with one or more green tea extracts prepared by other methods.

### Example 34. Green Tea Extract for Prevention of Influenza

This example is conducted to determine the effect of green tea extracts of the disclosure on preventing influenza infection in humans. The study is performed as a randomized, double-blind, placebo-controlled trial of a volunteer group, such as healthcare workers at a number for providing significant results, such as two hundred volunteers. It is preferably conducted over a seasonal time frame to cover development of influenza, such as for five to six months from October or November to the following April.

The treatment group receives capsules containing a standardized therapeutic dose of green tea catechins of the disclosure (for example, Phytofare® standardized to 378 mg catechins/day). The control group receives placebo (no catechins), and another control group can receive a dose of prior art green tea catechins (for example, Teavigo).

The primary outcome to be measured is the incidence of clinically defined influenza infection. Secondary outcomes that can be measured include laboratory-confirmed influenza with viral antigen measured by immunochromatographic assay, and the time for which the patient was free from clinically defined influenza infection, i.e., the period between the start of intervention and the first diagnosis of influenza infection, based on clinically defined influenza infection.

### Example 35. Green Tea Extracts to Reduce Transmission of Influenza Virus

The ability of green tea extracts of the disclosure to reduce physical transmission of influenza virus by skin-to-skin contact is evaluated using an artificial skin model. To set up the model, a support pad is placed over 13 ml of maintenance medium. Artificial skin cells, such as Neoderm® E cells, are placed on the support pad and incubated for 24 hours at 37° C. in a CO₂ atmosphere prior to performing the assays.

The Neoderm® E cells are infected with influenza virus to be tested, such as 1×10⁴ pfu of the X-31 ca virus (H3N2). After 45 minutes of shaken incubation at room temperature, each well is washed with PBS for viral titration (day 0). Minimal essential medium is then added to each well and incubated for 24 hours at 37° C. in a CO₂ atmosphere, then each well is washed with PBS for viral titration after incubation day (day 1).

To measure the effect of green tea extracts, a viral clearance assay is performed using the artificial skin cells. The skin cells are infected with 5×10⁴ pfu of the X-31 ca virus. After 45 minutes of shaken incubation at room temperature, each well is washed with Phytofare® or non-catechin control, or optionally one or more catechin standards such as EGCG. The plaque assay (pfu) is conducted for day 0. Minimal essential medium is then added to each well and incubated for 24 hours at 37° C. in a CO₂ atmosphere to further examine the presence of viruses. Each well is washed with PBS for viral titration after the incubation day (day 1).

Prior work by Shin, W.-J. et al. (Biosci. Biotechnol. Biochem. 76:581-584, 2012) demonstrated that a green tea bag solution had virucidal activity in this artificial skin model, supporting the use of the disclosed green tea extracts in hand-wash solutions, skin creams, and skin lotions for reducing person to person transmission of influenza virus.

### Example 36. Green Tea Extracts as Anti-Microbials for Treating Sepsis

Recent evidence suggests that EGCG can have an anti-microbial effect by altering microbial protein conformations and functions (Zhao, L. et al., Inflammation & Allergy—Drug Targets 12:308-314, 2013). To test the effect of green tea extracts of the disclosure, including Phytofare®, on bacteria involved in systemic infection (including sepsis and septic shock), the following experiments are conducted.

Murine macrophage-like RAW 264.7 cells are obtained from American Type Culture Collection (ATCC, Rockville, Md., USA), and cultured in Dulbecco's Modified Eagle's Medium (DMEM, Gibco, Grand Island, N.Y.), supplemented with 10% fetal bovine serum (FBS), 100 U/mL penicillin (Gibco), at 37° C. in a humidified 5% CO₂ atmosphere. Alexa Fluor 594-conjugated Staphylococcus aureus or Alexa Fluor 488-labeled Escherichia coli (Invitrogen, Carlsbad, Calif., USA) are re-suspended in 1 μl PBS, and added to macrophage cultures (at 1.0 μg/ml). Following incubation for 2 h, Phytofare® or control solution is added, and optionally one or more green tea standards are tested for comparison, such a (−)-Epigallocatechin Gallate (EGCG, 20 μM), and the cells are incubated for 30 min.

Following thorough washings, the intensity of fluorescence is estimated by fluorescence microscopy or flow cytometry, respectively. For flow cytometry, the fluorescence of phagocytozed bacteria is analyzed with a FACSCalibur instrument (Becton Dickinson) equipped with CellQuest software. For each sample, at least 1×10⁴ cells are collected and analyzed.

The effects of green tea extracts of the disclosure on the fluorescence of Alexa Fluor Dyes is measured as follows. Alexa Fluor 594 and Alexa Fluor 488 carboxylic acids (Invitrogen, Carlsbad, Calif., USA) are dissolved in 1×PBS at the concentration of 250 ng/ml. Bacteria-conjugated Alexa Fluor dyes are re-suspended into 1×PBS to generate bacterial suspension at the concentration of 40 μg/ml. To determine whether Phytofare® or catechin standards (such as EGCG) affects the intensity of Alexa Fluor dyes, the unconjugated or bacteria-conjugated fluorescence dyes are exposed to Phytofare®, EGCG (20 μM) or analogs for 30 min. The intensity of the fluorescence is measured using a fluorescence microscope (Carl Zeiss Microimaging) or a Fluorescence Spectrophotometer (F-7000, Hitachi High Technologies America, Inc.).

### Example 37. Effect of Green Tea Extracts on Systemic Inflammation

This experiment is performed to determine the effects of green tea extracts of the disclosure, including Phytofare®, on bacterial endotoxin-induced HMGB1 (high mobility protein group B1) release. HMGB1 is a late mediator of lethal systemic inflammation, and plays a role in the inflammatory destruction related to sepsis and other inflammatory conditions.

Murine macrophage-like RAW 264.7 cells are obtained for example from the American Type Culture Collection (ATCC, Rockville, Md.) and cultured in DMEM medium (Gibco BRL, Grand Island, N.Y.) supplemented with 10% fetal bovine serum and 2 mM glutamine. At 80-90% confluence, RAW 264.7 cells are washed twice with, and subsequently cultured in, serum-free DMEM medium before stimulation with bacterial endotoxin (lipopolysaccharide, LPS, E. coli 0111:B4, Sigma-Aldrich) alone, or in the presence of green tea extracts at selected concentrations. Exemplary concentrations include the equivalent of 75 ml green tea/person, or 10 μl/ml in assay culture.

At 16 hours after stimulation, levels of nitric oxide and HMGB1 in the culture medium are determined by Griess reaction and Western blot, respectively (Rendon-Mitchell B, et al., J. Immunol. 170:3890-3897, 2003).

In a previous study, Lipton green tea was reported to dose-dependently inhibit endotoxin-induced release of nitric oxide and HMGB1 (Chen, X. et al., Med Hypotheses 66:660-663, 2006). At a dose as low as 10 μl/ml (equivalent of 75 mls/person, assuming a total body weight of 75 kg, and blood volume of 7,500 mls), Chen reported that green tea almost completely abolished endotoxin-induced HMGB1 release. Even at concentrations that almost completely abrogated HMGB1 release, green tea did not exhibit any cytotoxicity to macrophage cultures, because cell viability, as assessed by trypan blue exclusion, was not reduced [92%, for control; versus 91%, in the presence of LPS+tea (10 μl/ml)]. The authors proposed that green tea might be beneficial for patients with systemic inflammation (such as endotoxemia and sepsis). The green tea extracts of the present disclosure with higher bioavailability are expected to yield even better outcomes that the green tea used in the previous study.

### Example 38. Green Tea Extracts to Improve Oral Health and Reduce Caries

This example is performed to study the effect of green tea extracts of the disclosure on bacterial growth on biofilm, as a model of oral health leading to caries formation.

Bacterial Strains. S. mutans UA159, a virulent cariogenic pathogen, is used for biofilm studies. The cultures are stored at −80° C. in tryptic soy broth containing 20% glycerol. Biofilm preparation is performed as follows. Hydroxyapatite disks (surface area of 2.7±0.2 cm²; Clarkson Chromatography Products Inc., South Williamsport, Pa., USA) are coated with filter-sterilized (0.22 μm; polyether sulfone low protein-binding filter; Millipore Co., Bedford, Mass., USA) clarified human whole saliva for 1 h at 37° C.; whole saliva is collected on ice from a donor following paraffin film chewing, and it is clarified by centrifugation (8,500 g, 4° C., 10 minutes) (Koo et al., Caries Res 34: 418-426, 2000).

Biofilms of S. mutans UA159 (ATCC 700610) are formed on saliva-coated hydroxyapatite (sHA) disks placed in a vertical position using a disk holder in ultrafiltered (Amicon 10-kDa molecular weight cutoff membrane; Millipore Co.) tryptone-yeast extract broth by addition of 30 m M sucrose at 37° C. and 5% CO² for 5 days (Koo et al., J. Dent. Res. 84: 1016-1020, 2005). During the first 24 hours, the organisms are grown undisturbed to allow initial biofilm formation; the biofilms (24 h old) are then treated twice daily (at 10 a.m. and 4 p.m.) until the 5th day of the experimental period (120-hour-old biofilms) with one of the following: (a) green tea extract such as Phytofare® (1.5 mg of green tea extract dry weight/ml); (b) 250 ppm of F (as sodium fluoride; positive control); (c) vehicle control (10% ethanol, v/v; negative control); and (d) optionally one or more green tea catechin standards, such as EGCG.

The biofilms are exposed to the treatments for 1 minute, dip-rinsed 3 times in sterile saline solution (to remove excess treatment agents or vehicle control) and transferred to fresh culture medium. The treatments and rinsing procedures are repeated 6 hours later. Each biofilm is exposed to the respective treatment a total of 8 times. Biofilm assays are performed in quadruplicate in at least 3 different experiments.

Biofilm analyses are performed as follows. At the end of the experimental period (120-hour-old biofilms), the biofilms are removed and subjected to sonication using three 30-second pulses at an output of 7 W (Branson Sonifier 150; Branson Ultrasonics, Danbury, Conn., USA) (Koo et al., J. Antimicrob. Chemother. 52:782-789, 2003). The homogenized suspension is analyzed for biomass (dry weight), total protein (by acid digestion followed by ninhydrin assay) (Moore, S. et al., J. Biol. Chem 211:907-913, 1954) and polysaccharide composition. The extracellular water-soluble and -insoluble polysaccharides, and intracellular iodophilic polysaccharides are extracted and quantified by colorimetric assays as detailed by Koo et al. (2003) and Duarte, S. et al. (Oral Microbiol. Immunol. 23:206-212, 2008).

Briefly, an aliquot (4 ml) of the biofilm suspension is centrifuged at 10,000 g for 10 min at 4° C. The supernatant is collected and the biofilm pellet resuspended and washed in the same volume of water; this procedure is repeated twice. All the supernatants are pooled and three volumes of cold ethanol added, and the resulting precipitate (or water-soluble polysaccharides) collected and washed with cold ethanol; the total amount of carbohydrate is determined by the phenol-sulfuric method (Dubois et al., Analytical Chem. 28: 350-356, 1956).

The biofilm pellet is dried in a Speed Vac concentrator and used for the determination of (i) extracellular insoluble polysaccharides and (ii) intracellular iodophilic polysaccharides. The insoluble polysaccharides are extracted using 1 N NaOH (1 mg of biofilm dry weight/0.3 ml of 1 N NaOH) under agitation for 2 h at 37° C. and quantified by the phenol-sulfuric method. The intracellular iodophilic polysaccharides are optionally extracted with hot 5.3 M KOH (0.8 mg of biofilm dry weight/ml of KOH) and quantified using 0.2% I₂/2% KI solution and glycogen as a standard, as described by DiPersio et al. (Infect. Immun. 10: 597-604, 1974). The pH of the culture medium is measured daily at specific time points (such as 60, 120 and 240 min after medium replacement) by a glass electrode (Futura Micro Combination pH electrode; 5 mm diameter; Beckman Coulter Inc., Calif., USA).

Endpoints indicating that Phytofare® and green tea extracts of the disclosure are effective in reducing biofilm, compared to vehicle control or fluoride treatments, include (1) reduced dry weight; (2) reduced insoluble glycans, by weight; and (3) higher pH, indicating reduction in acidity.

As evidence of an effect of green tea extracts on oral health, the dental caries inhibiting effect of an extract from Japanese green tea was studied both in vitro and in vivo by Otake S., et al., Caries Res. 25:438-431 (1991). The crude tea polyphenolic compounds (designated Sunphenon) from the leaf of Camellia sinensis were found to effectively inhibit the attachment of Streptococcus mutans strain JC-2 (serotype c) to saliva-coated hydroxyapatide discs. Sunphenon was also inhibitory to water-insoluble glucan formation from sucrose by crude glucosyltransferase of S. mutans JC-2 (c). Among the tea catechins tested, (−)-epigallocatechin gallate and (−)-epicatechin gallate showed the most potent inhibition of the glucosyltransferase activity. Significantly lower caries scores were observed in specific pathogen-free rats infected with S. mutans JC-2 (c) and fed a cariogenic diet and/or drinking water containing 0.05% Sunphenon as compared with control rats not receiving polyphenolic compounds.

Thus, there is justification and rationale for use of the green tea extracts of the disclosure, including Phytofare®, to reduce oral biofilm as part of a program to improve oral health and reduce caries development. The green tea extracts can be provided in any suitable form to deliver an appropriate dose to the oral cavity, including toothpaste or gel, oral rinse, chewing gum, or lozenge.

### Example 39. Effect of Green Tea Extracts on Anti-HBV Activity in HepG2.117 Cells

This example is performed to measure the anti-HBV (hepatitis B virus) activity of EGCG in HepG2.117 cells, an inducible HBV-replicating cell line (Su, D. et al., J. Hepatol. 45: 636-645, 2006).

HepG2.117 cells are cultured in DMEM medium (Invitrogen) supplemented with 10% fetal bovine serum (Gibco), 100 U/mL penicillin and 100 μg/mL streptomycin (Sigma). When needed, doxycycline (Sigma) is routinely added at 1.5 μg/mL to suppress HBV pgRNA transcription.

Green tea extract treatment and cytotoxicity assay. Three days after the removal of doxycycline, HepG2.117 cells are seeded into 24-well plates. Twenty-four hours later, cells are treated with fresh DMEM medium containing various concentrations of green tea extract such as Phytofare®, and optionally with catechin standards such as EGCG. Control cell cultures receive no green tea or catechin treatment. Optionally, cell cultures are treated with a known anti-viral pharmaceutical, such as lamivudine (NIH AIDS Research and Reference Reagent Program, Rockville, Md., USA). The treatment-containing media are replaced each day for 3 days.

The medium is then removed, and attached cells are used for toxicity analysis, such as using the 3-(4,5-Dimethylthiazol-2-yl)-2,5-diphenyltetrazolium bromide (MTT) assay. Cells cultured with DMEM medium without green tea extract or catechins are used as a negative control, and can be arbitrarily designated as 1 for data analysis. Preferably, three independent experiments are performed and standard deviation is calculated.

### Example 40. HCV: Effect of Green Tea Extracts on Ava5 Cells

This example is performed to evaluate the cytotoxicity of green tea extracts of the disclosure on Ava5 cells. Ava5 cells, human hepatoma cells (Huh-7) cells containing the subgenomic HCV genotype 1b replicon, (Blight, K. J. Science 290: 1972-1974, 2011) are cultured in DMEM with 10% heat-inactivated FBS, 1% antibiotic-antimycotic solution, 1% nonessential amino acids, and 1 mg/ml G418 (antibiotic) and are incubated at 37° C. with 5% CO₂ supplement.

For comparison with the green tea extracts of the disclosure, one or more catechin standards can be used. For example, (+)-CAT, (2)-CAT, (+)-EC and (2)-EC with 98% purity can be obtained from Kishida Chemical Co., Ltd.; these compounds are isolated from green tea leaves. All tested catechin compounds are preferably stored at 10 mM in 100% dimethylsulfoxide (DMSO). The final concentration of DMSO in all reactions is preferably maintained constantly at 0.1% in each experiment.

To perform the cytotoxicity assays, Ava5 cells are seeded in 96-well plates at a density of 5×10³ cells per well and then incubated with compounds at various concentrations for three days. The cell viability can be determined by the colorimetric 3-(4,5-dimethylthiazol-2-yl)-5-(3-carboxymethoxyphenyl)-2-(4-sulfophenyl)-2H-tetrazolium (MTS) assay (Promega Corporation, Madison, Wis.) as described (Lee, J. C. et al., Antiviral Res 89:35-42, 2011).

### Example 41. Green Tea Extracts to Reduce UV-Related Skin Injury

The goal of this example is to evaluate the effect of green tea extracts of the disclosure on parameters associated with acute UV injury, in human volunteers.

Areas of skin of normal volunteers are treated with a green tea extract of the disclosure such as Phytofare®, or with one or more green tea catechin standards. Thirty minutes later, the treated sites are exposed to a 2 minimal erythema dose solar simulated radiation. UV-treated skin is examined clinically for at least parameter of UV-related injury, including for example UV-induced erythema, histologically for the presence of sunburn cells or Langerhans cell distributions, or biochemically for UV-induced DNA damage.

In a previous study (Elmets, C. A. et al., J. Am. Acad. Dermatol. 44:425-432, 2001), (−)-epigallocatechin-3-gallate (EGCG) and (−)-epicatechin-3-gallate (ECG) polyphenolic fractions were most efficient at inhibiting erythema, whereas (−)-epigallocatechin (EGC) and (−)-epicatechin (EC) had little effect. On histologic examination, skin treated with green tea extracts reduced the number of sunburn cells and protected epidermal Langerhans cells from UV damage. Elmets et al. also reported that green tea extracts reduced the DNA damage that formed after UV radiation.

Application of green tea extract of the disclosure to the skin is therefore expected to result in inhibition of the erythema response evoked by UV radiation. Green tea extracts of the disclosure are expected to be effective chemopreventive agents for many of the adverse effects of sunlight on human health and may thus serve as natural alternatives for photoprotection.

### Example 42. Ocular Anti-Oxidant Use of Green Tea Extracts

This example is performed to study the pharmacokinetics of catechins and oxidation status in rat eye after oral administration of green tea extracts of the disclosure and controls. Rats, such as Sprague-Dawley rats, are fed green tea extracts and sacrificed at different time intervals. The eyes are dissected into cornea, lens, retina, choroid-sclera, vitreous humor, and aqueous humor for analysis of catechins and 8-epi-isoprostane by HPLC-ECD and GC-NCI-MS, respectively. Catechin distribution in eye tissues is studied.

A prior investigation (Chu, K. O. et al., J. Agric. Food Chem., 58:1523-1534, 2010) reported that gallocatechin was present at the highest concentration in the retina, 22729.4±4229.4 pmol/g, and epigallocatechin in aqueous humor at 602.9±116.7 nM. The authors reported that the corresponding area-under-curves were 207,000 pmol×h/g and 2035.0±531.7 nM×h, respectively, and the time of maximum concentration of the catechins varied from 0.5 to 12.2 h. Significant reductions in 8-epi-isoprostane levels were found in the compartments except the choroid-sclera or plasma, which according to Chu et al. indicated antioxidative activities of catechins in these tissues.

### Example 43. Treatment of Dry Eye Disease Using Green Tea Extracts

This example is performed to study the efficacy of topical green tea extracts for the treatment of dry eye disease. Seven- to eight-week-old female C57BL/6 mice are housed in the controlled environment chamber to induce dry eye disease. Topical 0.01% or 0.1% green tea extract, or vehicle as control, is applied to the eyes of the mice with dry eye disease. Corneal fluorescein staining and the number of corneal CD11b+ cells are assessed in the different groups.

Expression of interleukin-1β, tumor necrosis factor-α, chemokine ligand 2, and vascular endothelial growth factor (VEGF)-A/C/D is optionally evaluated by real-time polymerase chain reaction in the corneas at day 9. Corneas are stained for lymphatic vessel endothelial hyaluronan receptor (LYVE)-1 to evaluate lymphangiogenesis, and the terminal transferase dUTP nick end labeling (TUNEL) assay can be used to evaluate apoptosis of corneal epithelial cells.

In a previous study (Lee, H. S. et al., Cornea 30:1465-1472, 2011), treatment with 0.1% EGCG showed a significant decrease in corneal fluorescein staining compared with the vehicle (24.6%, P=0.001) and untreated controls (41.9%, P<0.001). A significant decrease in the number of CD11b+ cells was observed in 0.1% EGCG-treated eyes, compared with the vehicle in the peripheral (23.3%, P=0.001) and central (26.1%, P=0.009) corneas. Treatment with 0.1% EGCG was associated with a significant decrease in the corneal expression of interleukin-1β (P=0.029) and chemokine ligand 2 (P=0.001) compared with the vehicle, and in VEGF-A and VEGF-D levels compared with the untreated group (P=0.007 and P=0.048, respectively). Lower dose EGCG (0.01%) also showed a decrease in inflammation at the molecular level but no significant changes in the clinical signs of DED. No cellular toxicity to the corneal epithelium was observed with 0.01% or 0.1% EGCG.

Topical green tea extract treatment is expected to reduce the clinical signs and inflammatory changes in dry eye disease by suppressing the inflammatory cytokine expression and infiltration of CD11b+ cells in the cornea.

### Example 44. Phytofare® Catechin Complex Bioavailability Studies: Cross-Over Study Comparing the Bioavailability Phytofare® Against Phytofare® Pheroid®Catechin Complex and a Generic Green Tea Extract

Objective of Example 44.

The objective of this example is to compare the bioavailability of Phytofare® against Phytofare® Pheroid®Catechin Complex and a generic green tea extract, specifically, to evaluate the comparative bioavailability of epigailocatechin, epicatechin, epigallocatechin gallate, epicatechin gallate, catechin gallate, gallocatechin and total catechin.

This example describes a single-center, crossover, 24-hour bioavailability study with three arms. Human subjects are enrolled after undergoing a screening visit and passing eligibility criteria. Subjects act as their own control and receive a generic green tea extract (Arm 1), a Phytofare® Catechin Complex (Arm 2), and a Phytofare® Pheroid®Catechin Complex (Arm 3) for four days to establish a steady state. Blood plasma levels will be determined on day four with each arm of the study being separated by at least 14 days. 14 days is a very conservative wash-out period, as evidence from Williamson and Manach, 2005 indicates that catechins generally have an elimination half life of 2-3 hours and a meta-analysis of 97 studies undertaken by Manach et al., (2005) also describes the elimination half-lives of epicatechin, EGC and EGCG being 2.5±0.4, 2.3±0.2 and 3.5±0.3 hours respectively. An analysis undertaken by Moruisi in 2008 determined that EGCG that had been encapsulated in the Pheroid®delivery system had an elimination half-life of 2.6 hours; however, steady state levels of ECGC were still not reached after 8 hours.

To evaluate bioavailability, epigallocatechin, catechin, epicatechin, epigallocatechin gallate, epicatechin gallate, catechin gallate, gallocatechin and total catechin will be analyzed in plasma samples taken pre-dose and again at 30 minutes, 60 minutes, 90 minutes, 120 minutes, 180 minutes, 5 hours, 8 hours, 12 hours and 24 hours postdose. Standardized meals low in catechins and void of caffeine are provided. Breakfast will be provided after the 1-hour and 24 hour sampling and lunch will be provided after the 5-hour sampling with dinner after the 12 hour sampling. Subjects will be provided with the same meals in the clinic on each test day.

The period from screening to completion of the collection of biological samples will be approximately four months. Adverse events will be assessed at each study visit as well as on the first day on taking the capsules. Liver function of the subjects will be monitored at the screening phase and after each arm of the study.

The outputs of the study will include the concentration-time curves (AUC) for plasma epigallocatechin, epicatechin, epigallocatechin gallate, epicatechin gallate, catechin gallate, gallocatechin and total catechin which will be determined by LC-MS-MS.

Sample Preparation will optionally be done by protein precipitation reactions. The QTRAP 4000 LC MS/MS system has a very sensitive detection system and therefore this method can be used. SPE can also be used alternatively. The method will be validated in terms of specificity, lower limit of quantification (LLOQ), linearity, matrix effect, accuracy and precision. Internal Standards will be ethyl gallate, ecopoletin or myricetin.

Commercially available columns are used, such as Phenomenex Kinetex C18, 2.6 pm, 100A, 30×2.1 mm and Phenomonex Kinetex C18 2.6 pm, 100A, 100×2.1 mm since literature indicated the use of a Eclipse plus C18 (4.6 mm×100 mm, 1.8 pm) and a Phenomonex Luna C18 (2.0 mm×50 mm, 5 pm) (described in Dalluge & Nelson, 2000; Zhang et al. 2012; Bilbao, et al. 2007).

Additional endpoints include time at maximum concentration (Tₘₐₓ) and maximum concentration (Cₘₐₓ) for plasma epigallocatechin, catechin, epicatechin, epigallocatechin gallate, epicatechin gallate, catechin gallate, gallocatechin and total catechin.

This study will include a group of adult volunteers who meet inclusion criteria as follows: Male or female age 18 to 65 years; healthy as determined by laboratory results and medical history; agree to avoid foods/beverages high in catechins including tea and tea related beverages for 48 hours prior to and during each test day; agree to avoid caffeine and alcohol for 24 hours prior to and during each test phases; and have given voluntary, written, informed consent to participate in the study.

Subjects will be administered in the mornings for 4 days of the investigational formulations and blood taken on predetermined times on day four. A washout period of at least 14 days is required before each arm. Subjects will be instructed to take two capsules in the clinic on an empty stomach each of the four days. The time of dose will be recorded and the timing of blood draws will be based on the dose time. Subjects will washout for a minimum of 14 days prior to each test period.

On the test day, a dietary check list will be reviewed. Seated resting blood pressure, heart rate and temperature will be measured. Seated resting blood pressure, heart rate and temperature will be measured. Concomitant medication, fasting time and illness will be checked by completing a questionnaire. A vein catheter will be inserted by a medical practitioner. Pre-dose, fasting blood samples will be taken for plasma epigallocatechin, catechin, epicatechin, epigallocatechin gallate, epicatechin gallate, catechin gallate, gallocatechin and total catechin analysis. The participant will then be given two capsules at time 0 with water. Blood samples will be taken again at 30, 60, 90, 120 and 180 minutes, and 5, 8, 12 and 24 hours post-dose for analysis of plasma epigallocatechin, catechin, epicatechin, epigallocatechin gallate, epicatechin gallate, catechin gallate, gallocatechin and total catechin analysis.

Breakfast will be provided after the 1-hour and 24-hour sampling and lunch will be provided after the 5-hour and dinner after the 12-hour sampling. Breakfast, lunch and dinner meals will be provided by the clinic. Participants will remain in the clinic during the study visit from pre-dose until the 24-hour samples are collected. Participants will be allowed to watch television, use computers/laptops, read, talk, play video or board games, or sleep. The catheter will be removed and participants will be allowed to leave the clinic after the 12-hour post-dose blood sample. The participants must return for the 24 hour collection.

Participants will return to the clinic to begin the next test period. The next visit will be scheduled for at least 14 days after the last test day at the same time of day (in the morning, fasting 8-hours). A plus three day window (+3 days) will be allowed for scheduling issues.

Laboratory Analysis. Blood samples are drawn according to the study protocol timeline. A duplicate plasma sample will be collected during the plasma collection step in order to perform or repeat laboratory tests if needed.

At screening, whole blood will be collected into 4 ml EDTA tubes for CBC analysis. Serum will be generated from blood collected into 5 ml SST tubes for electrolytes, creatinine, AST, ALT, GGT and bilirubin. Participants should fast for a minimum of 8-hours prior to each blood profile day. Whole blood will be collected into 6 ml heparin tubes pre-dose, 30, 60, 90, 120 and 180 minutes, and 5, 8, 12 and 24 hours post-dose for catechin analysis (epigallocatechin, catechin, epicatechin, epigallocatechin gallate, epicatechin gallate, catechin gallate, gallocatechin and total catechin) by LC-MS-MS.

At the end of each study visit, 4 ml EDTA tubes will be collected at 24 hours post-dose for CBC analysis and serum will be generated from blood collected into 5 ml SST tubes for electrolytes, creatinine, AST, ALT, GGT and bilirubin.

At each time point, 2 aliquots of plasma (heparin), 500 pl each, will be transferred to storage tube. The plasma will be stored at −80° C. until analysis.

Data analysis can be performed using methods standard in the art, for example, line graphs showing the mean concentrations of plasma epigallocatechin, catechin, epicatechin, epigallocatechin gallate, epicatechin gallate, catechin gallate, gallocatechin and total catechin over time will be shown for each formulation. Bioavailability parameters, including the area under the curve (AUC), the maximum observed concentration (Cₘₐₓ) and time of maximum concentration (Tₘₐₓ) for the three study formulations will be calculated. Descriptive statistics, including means and standard deviations will be calculated for each formulation. Repeated measures analysis of variance will be used to compare the formulations with respect to these endpoints. Comparison of AUC will be performed on log transformed data. Participants withdrawing prior to completing all three periods of this cross over study will be excluded from the repeated measures analysis of variance.

Probability values less than 0.05 will be considered to be statistically significant. Effect sizes will be calculated. The statistical analysis will be performed using industry recognized statistical software such as R, SAS or SPSS.

Results Obtained from Practice of Example 44.

The description of Example 44 above contains guidelines. Following these guidelines, a green tea extract of the disclosure, referred to as Phytofare® Catechin Complex, was found to have a significantly higher degree of bioavailability, i.e. the level of catechin absorption and retention in the blood stream, than generic green tea extracts, as described below.

When comparing a generic green tea extract against Phytofare®, the results showed a ten-fold increase in all eight catechins in the blood (including EGCG) where the generic showed only two catechins. The absorption of Phytofare® was five times greater than with the generic while the lifespan of the catechins in the blood was doubled in the case of Phytofare®, resulting in ten times greater overall bioavailability of the Phytofare® extract.

Previous testing of green tea extracts has shown that catechin concentrations peak 1 to 2 hours after ingestion and gradually reduce to undetectable levels after 24 hours. An extract that allows the body to experience more of the health benefits of green tea is expected to have significant advantages over traditional green tea products where most of the catechins are lost through metabolization before they enter the bloodstream.

The non-randomized study used 27 human subjects who received generic green tea extract for four days and then had their blood plasma analyzed for a variety of catechins pre-dose and then at 30 minutes, 60 minutes, 90 minutes, 120 minutes, 180 minutes, 5 hours, 8 hours, 12 hours and 24 hours post-dose. After a 14-day washout period, the study subjects received a Phytofare® Catechin Complex for four days and had their blood plasma analyzed at the same intervals.

The third phase of the clinical study further examined the bioavailability of Phytofare® entrapped in Pheroid®, described in detail in the disclosure above. In a study of oral administration to human subjects of three green tea catechin compositions, EGCG (Epigallocatechin gallate), the most dominant catechin, was found at a tenfold higher level in the blood than in the generic green tea group, and eleven times higher when Phytofare® was delivered in Pheroid.

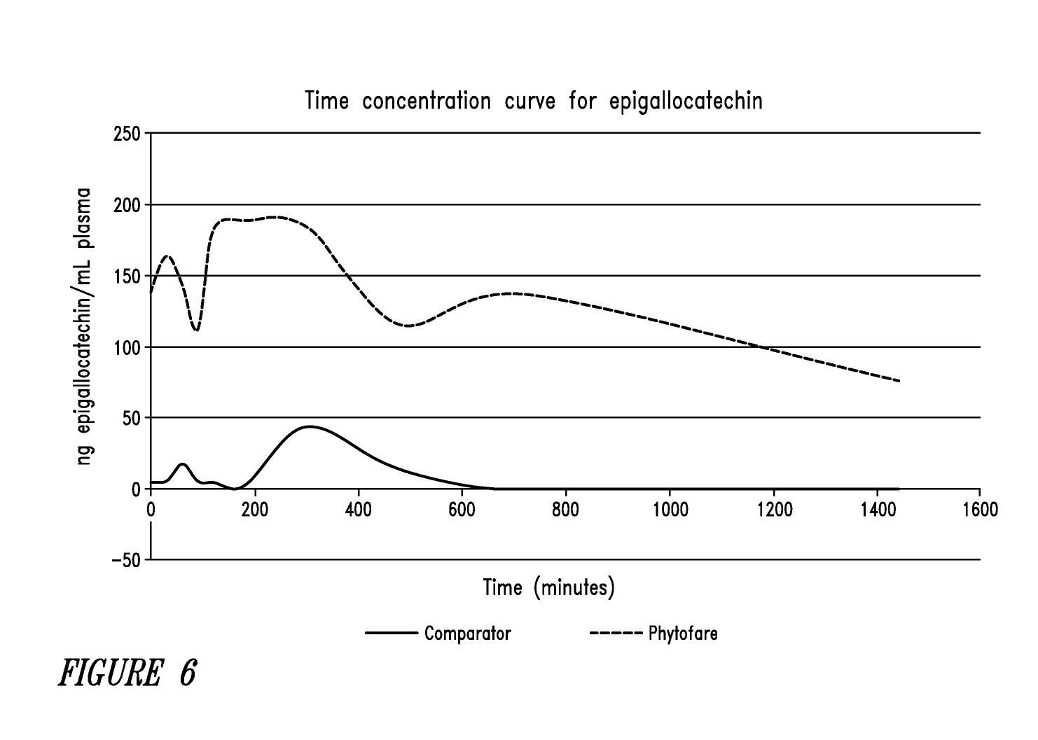

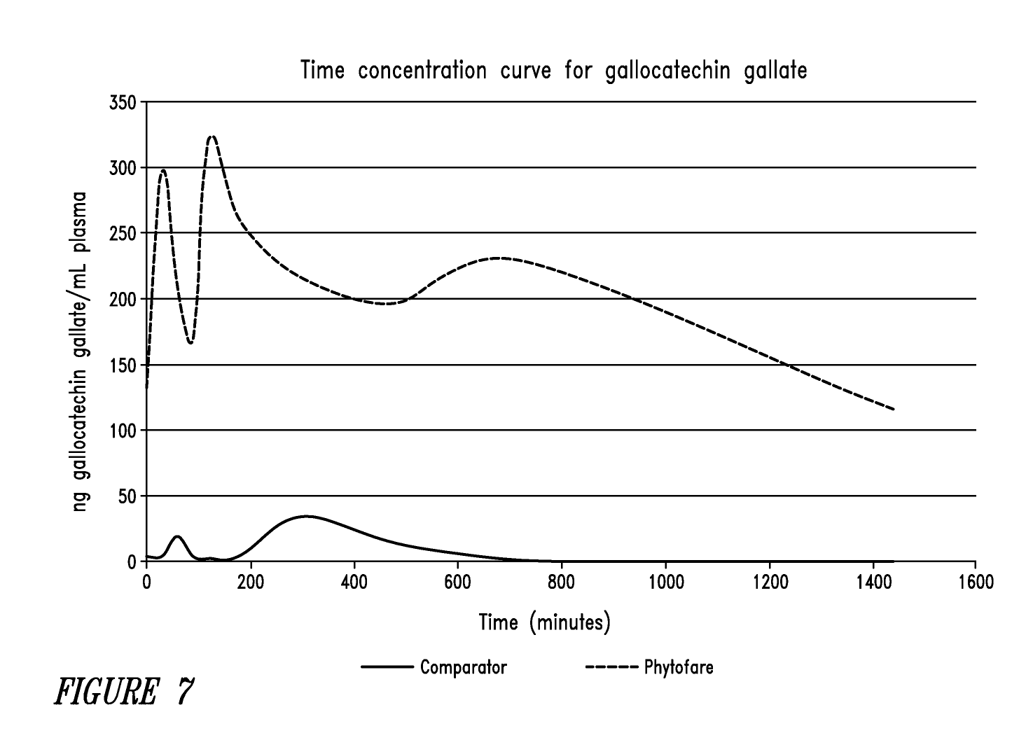

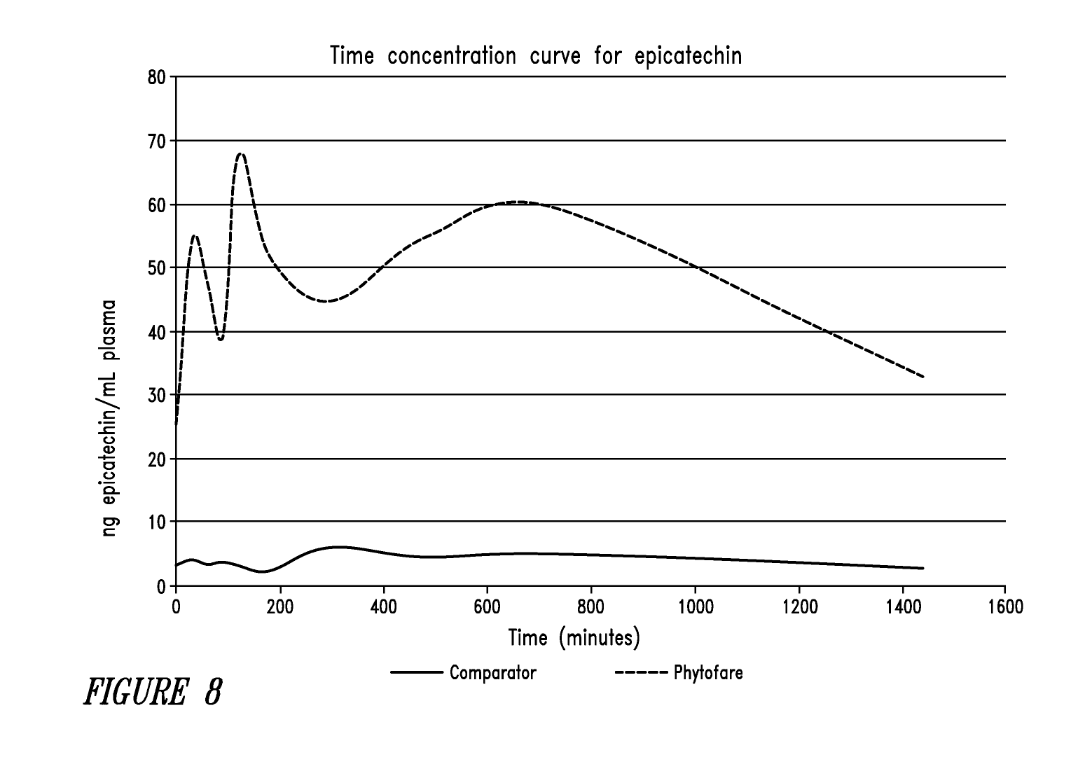

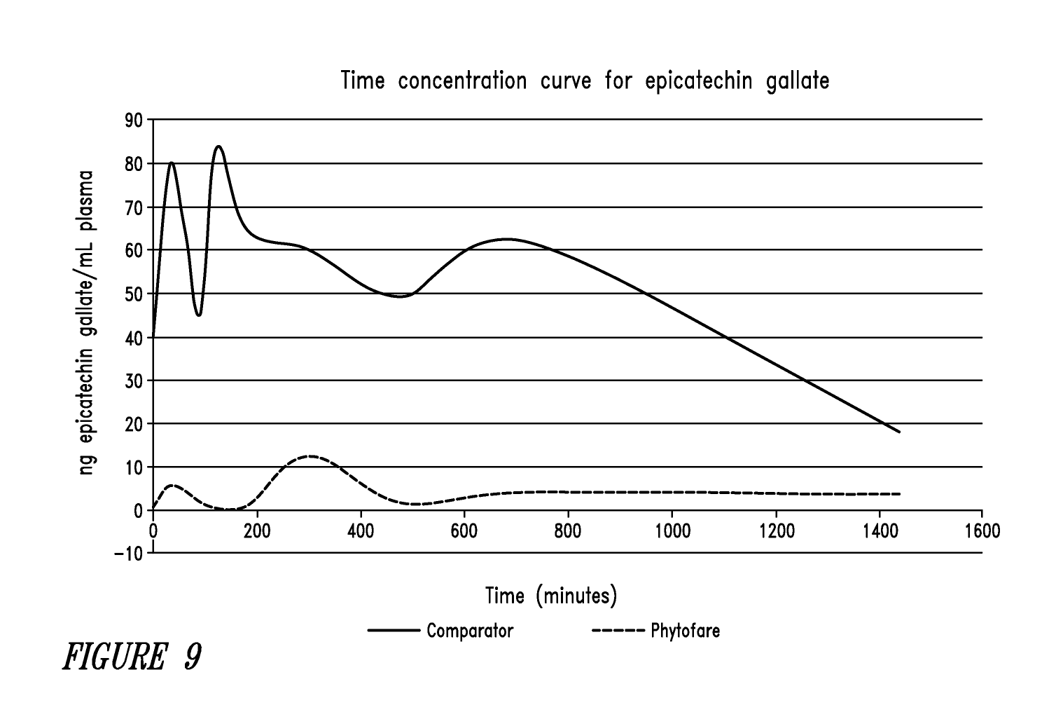

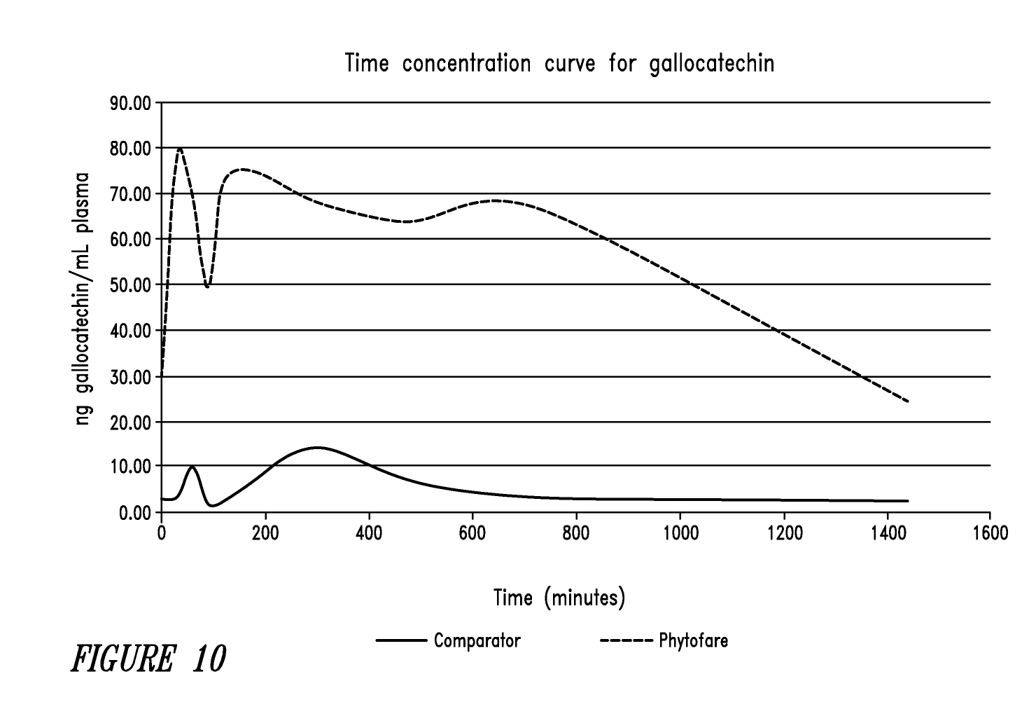

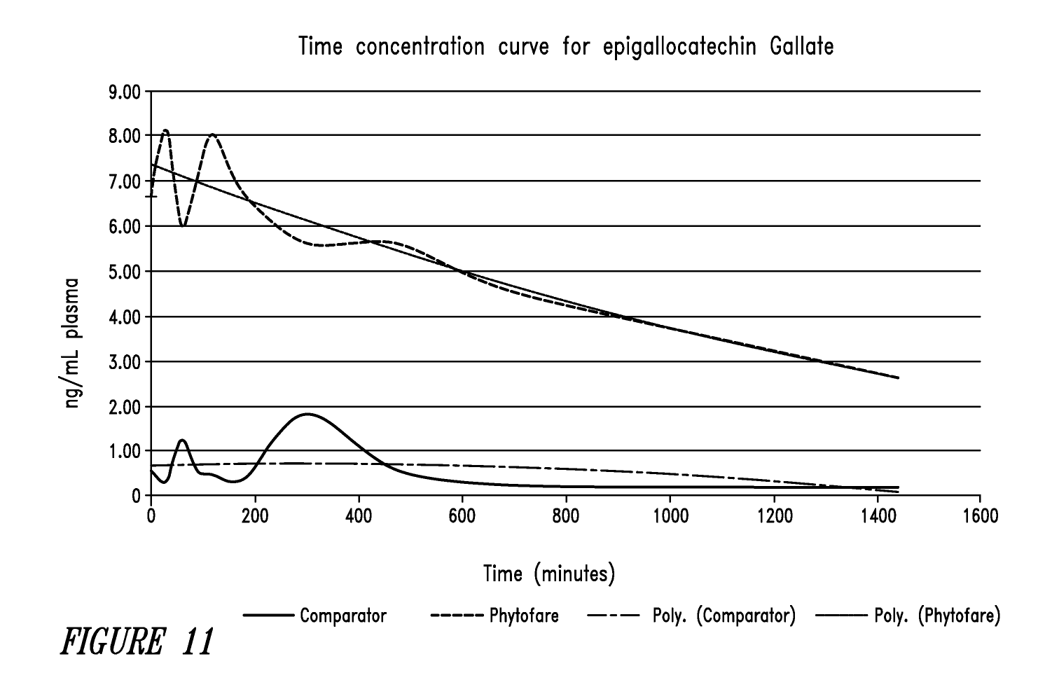

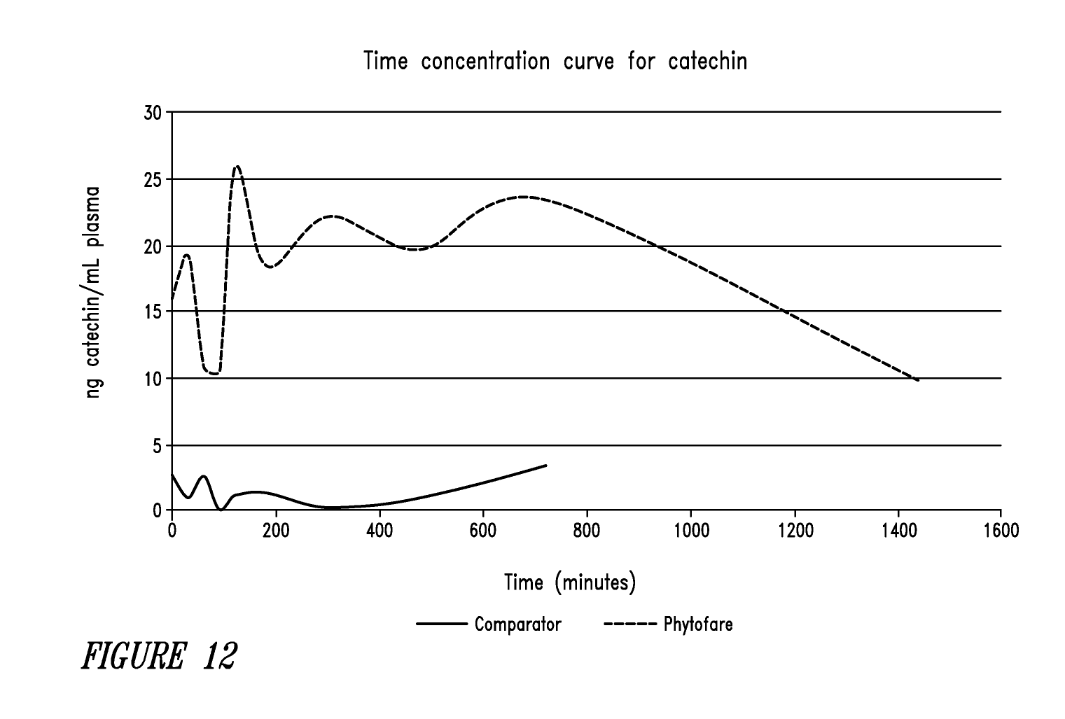

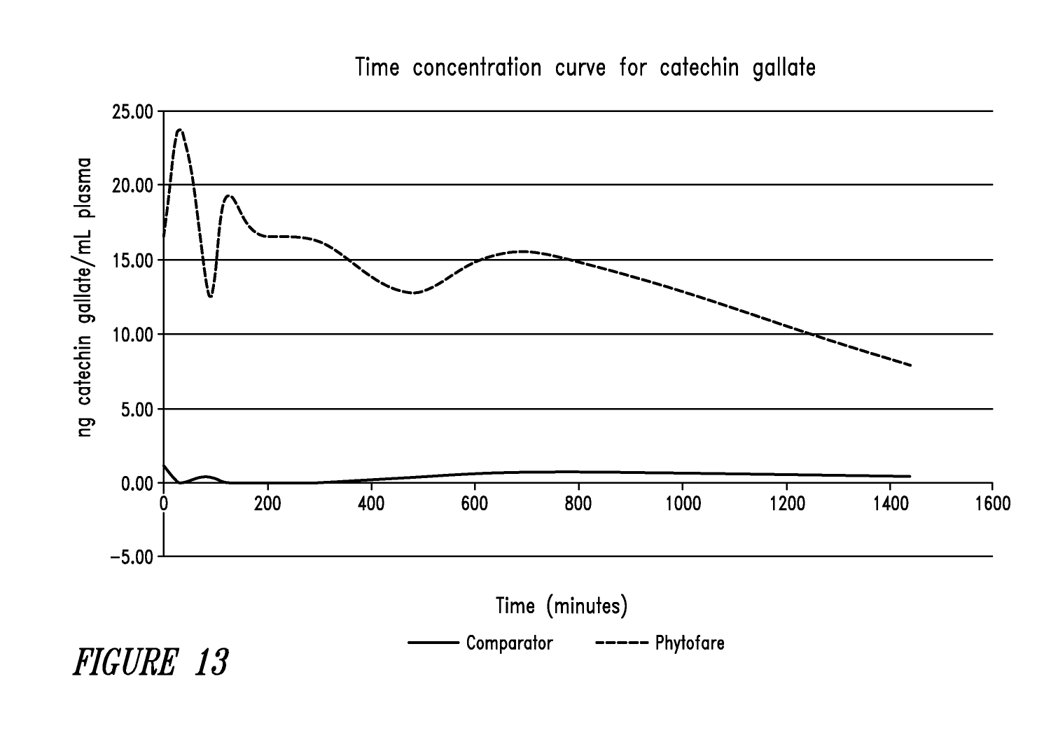

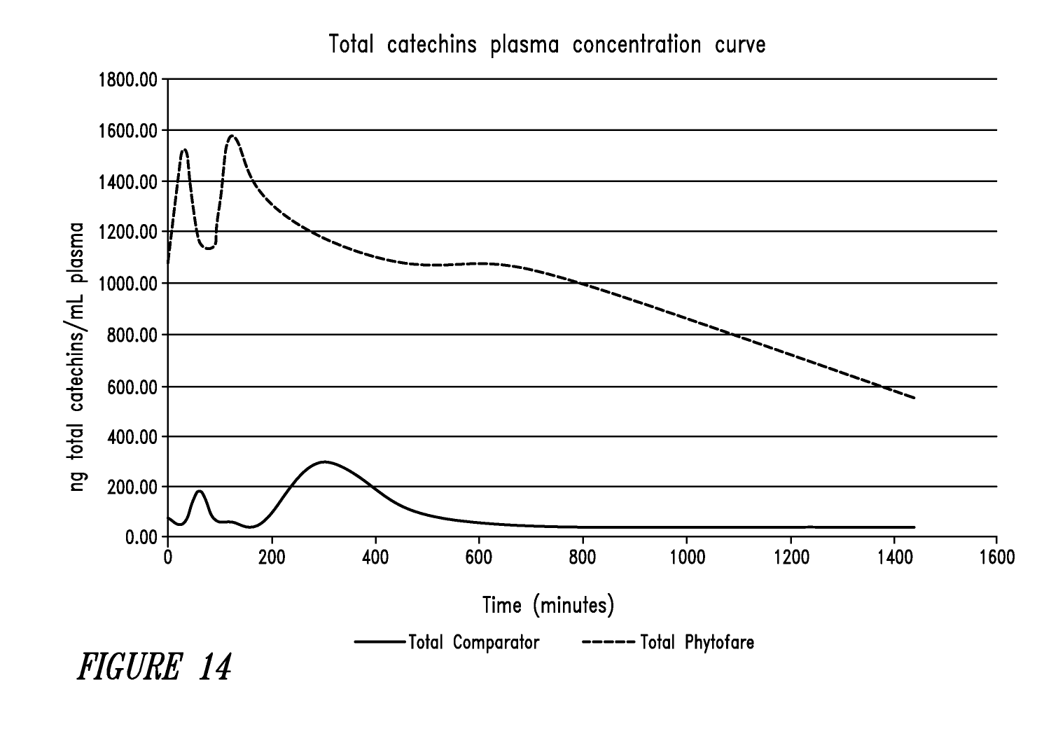

The results are shown in further detail in the Figures. FIGS. 6-13 show time concentration curves for, respectively, epigallocatechin, gallocatechin gallate, epicatechin, epicatechin gallate, gallocatechin, epigallocatechin gallate, catechin, and catechin gallate. FIG. 14 shows a total catechins plasma concentration curve.

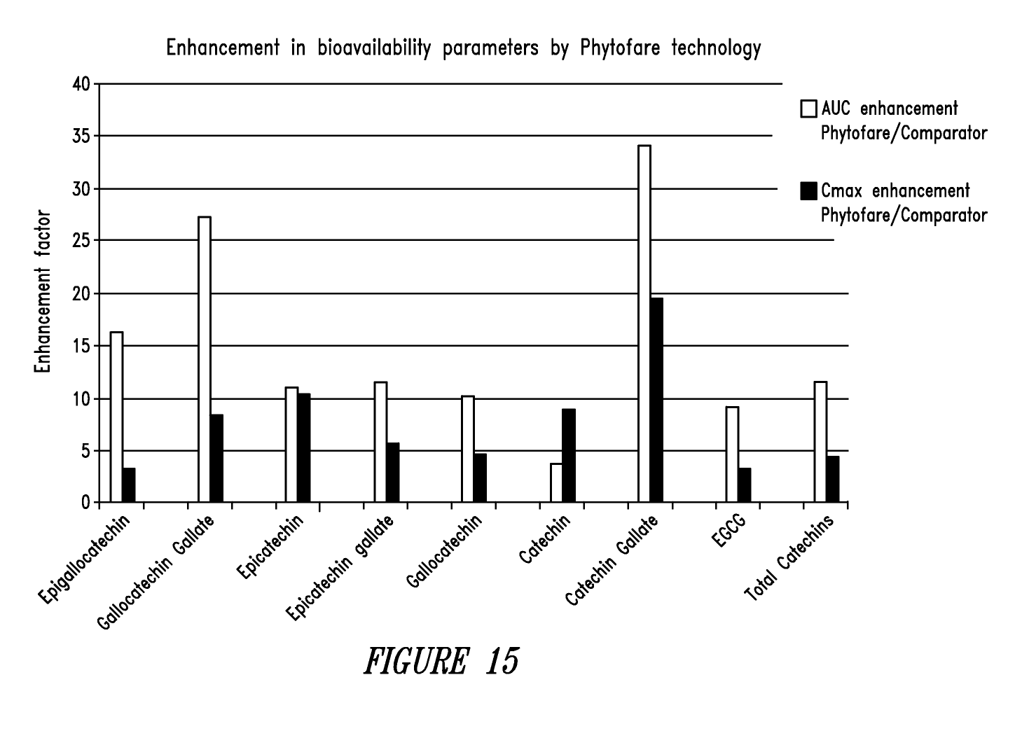

FIG. 15 shows the calculated enhancement of bioavailability, in which the open bars show AUC (area under the curve) and closed bars show Cₘₐₓ enhancement for Phytofare® vs. comparator. There is a statistically significant difference between the enhancement factors of the AUC and the Cₘₐₓ as determined by the T-test (p=0.025978394; one tailed). Based on this result, the following conclusions are drawn:

1. There is no obvious correlation between the AUC and Cₘₐₓ in the enhancement factors of the various catechins.

2. The enhancement in AUC in the case of the Phytofare® extract cannot be explained by the increase in Cₘₐₓ, which indicates that the compound exposure is enhanced by less clearance and metabolism.

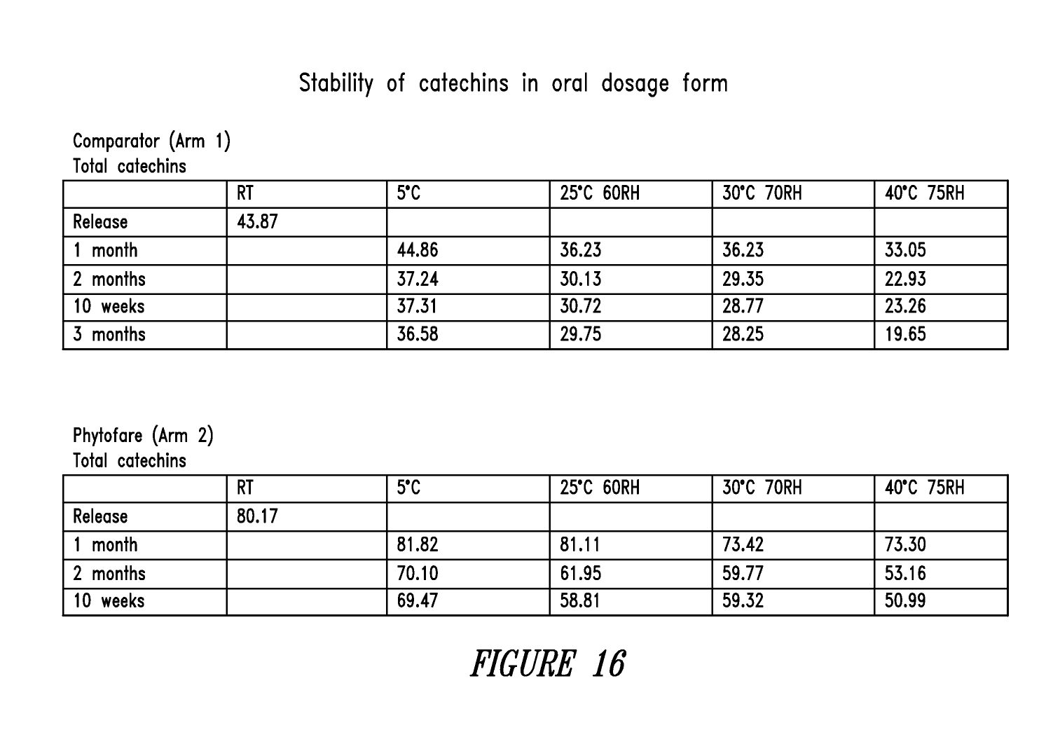

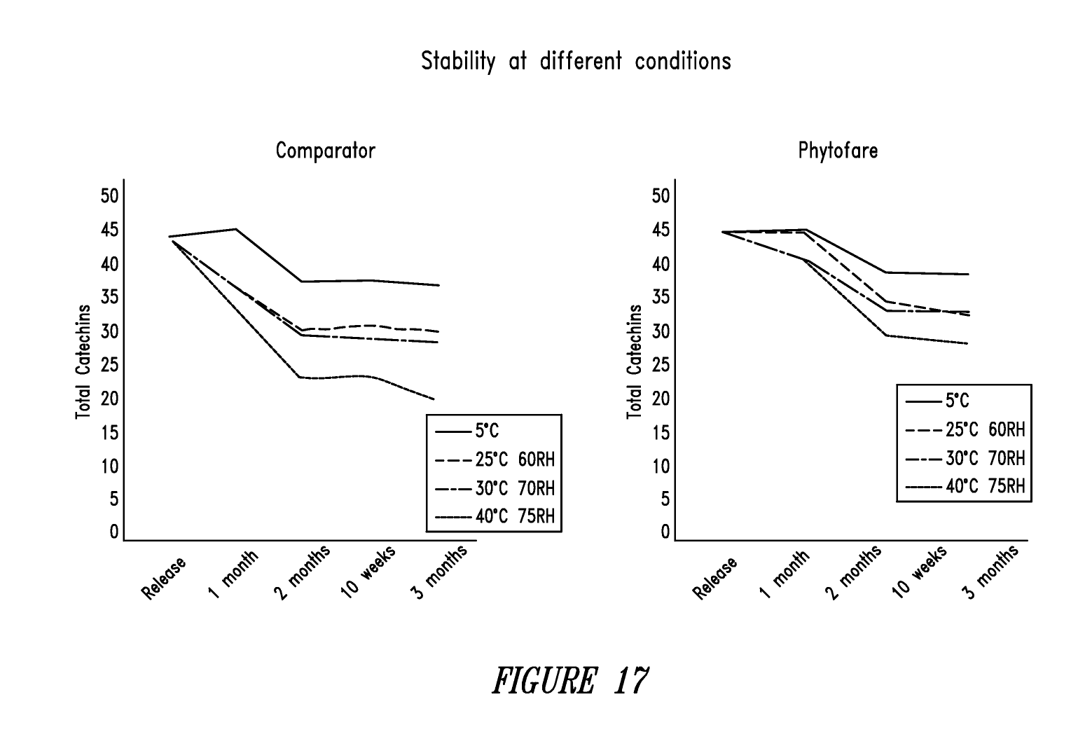

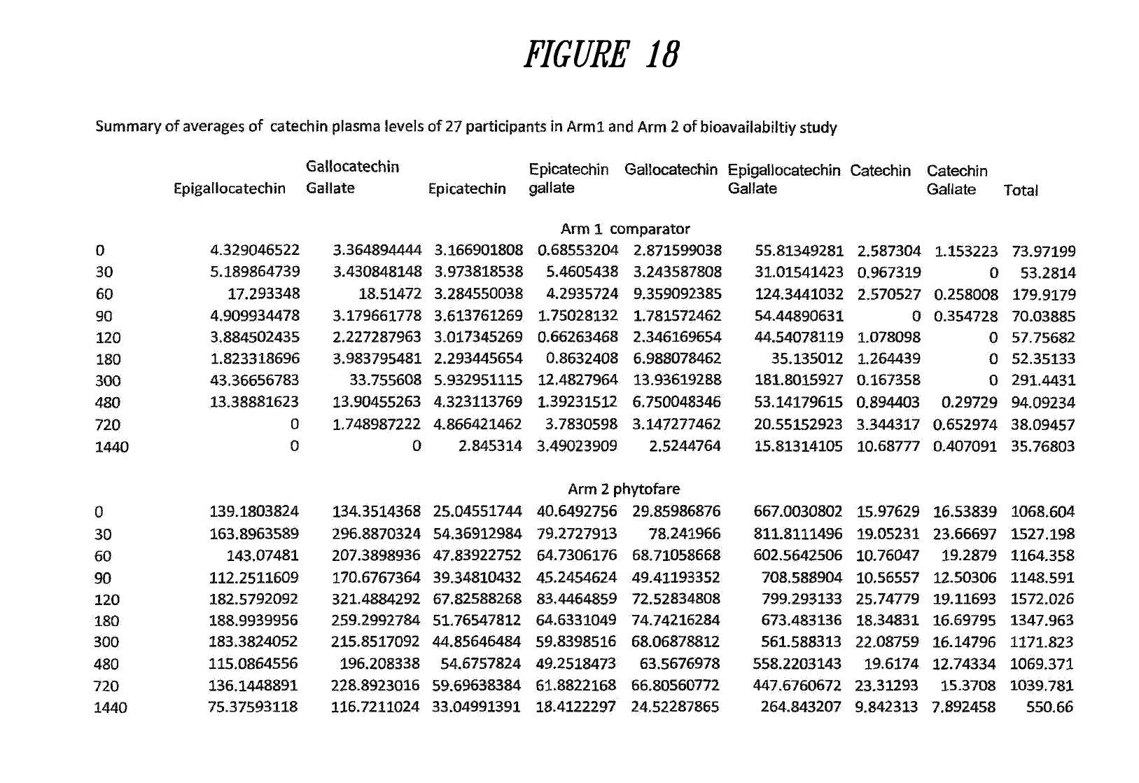

FIG. 16 is a table showing the stability of catechins in oral dosage form. Top table, Comparator, total catechins. Bottom table, Phytofare®, total catechins. FIG. 17 depicts stability of total catechins at different conditions, comparing the Comparator with Phytofare®. FIG. 18 is a table showing a summary of averages of catechin plasma levels of 27 participants in Arm 1 (Comparator) and Arm 2 (Phytofare®) of the bioavailability study described in Example 44.

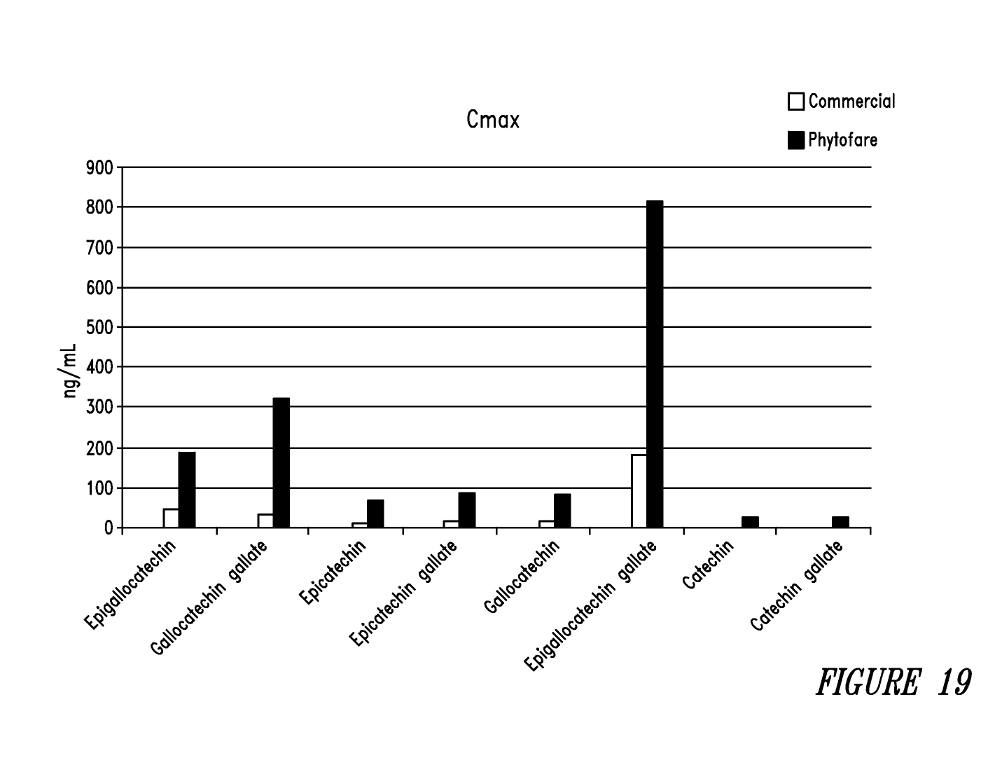

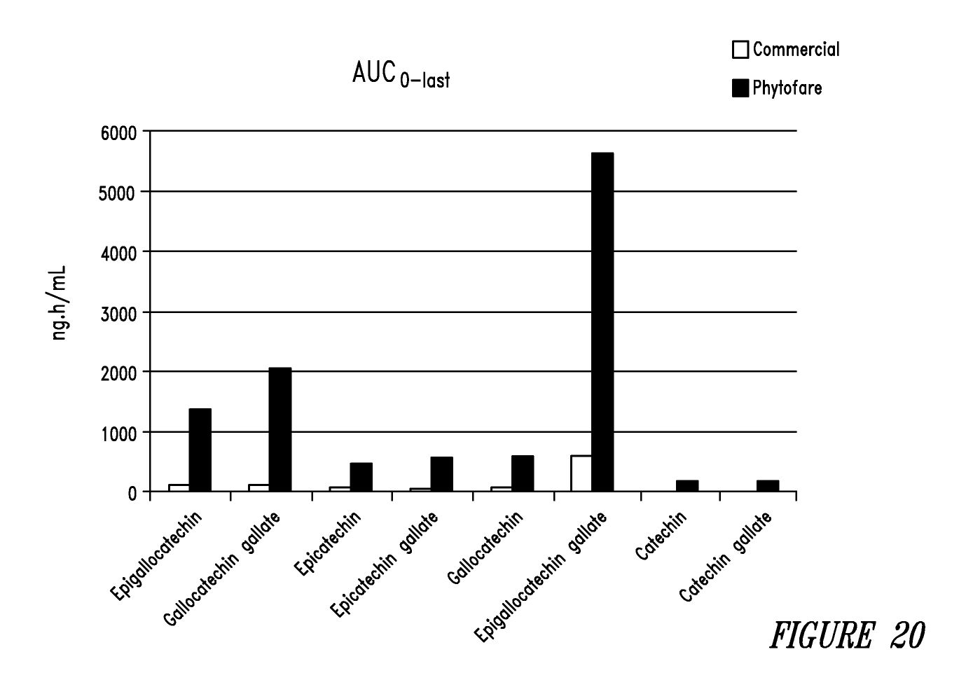

FIG. 19 shows the comparative peak average concentrations (Cₘₐₓ) of catechins attained in the plasma of participants after oral administration of the commercial and Phytofare® extracts. FIG. 20 shows the comparative areas under the curve (AUC) calculated for the catechins after oral administration of commercial and Phytofare® products, using Prism Graphpad after normalization of the data.

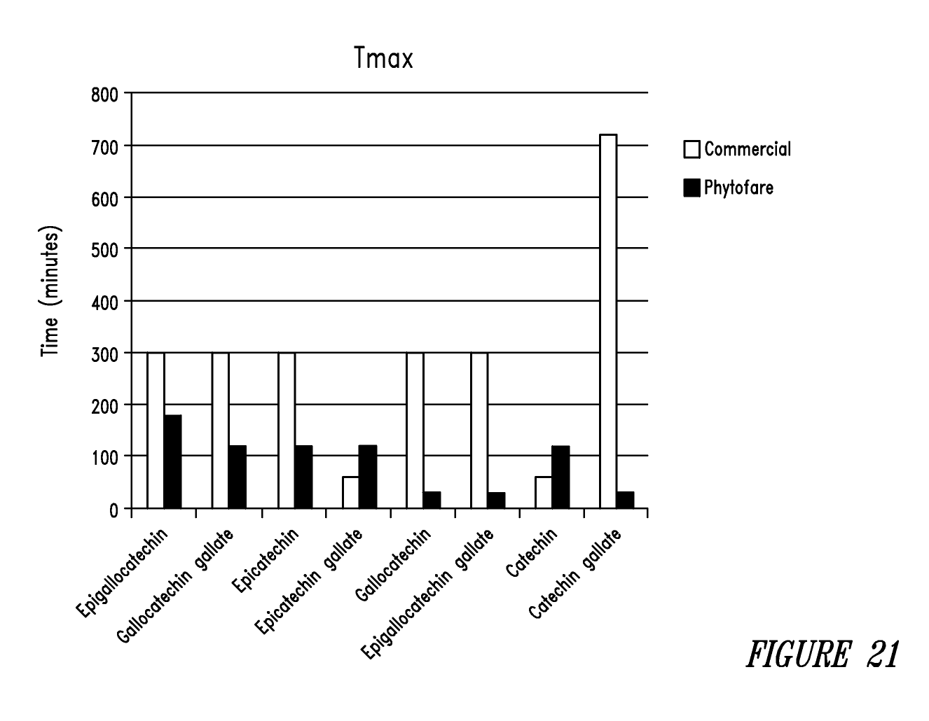

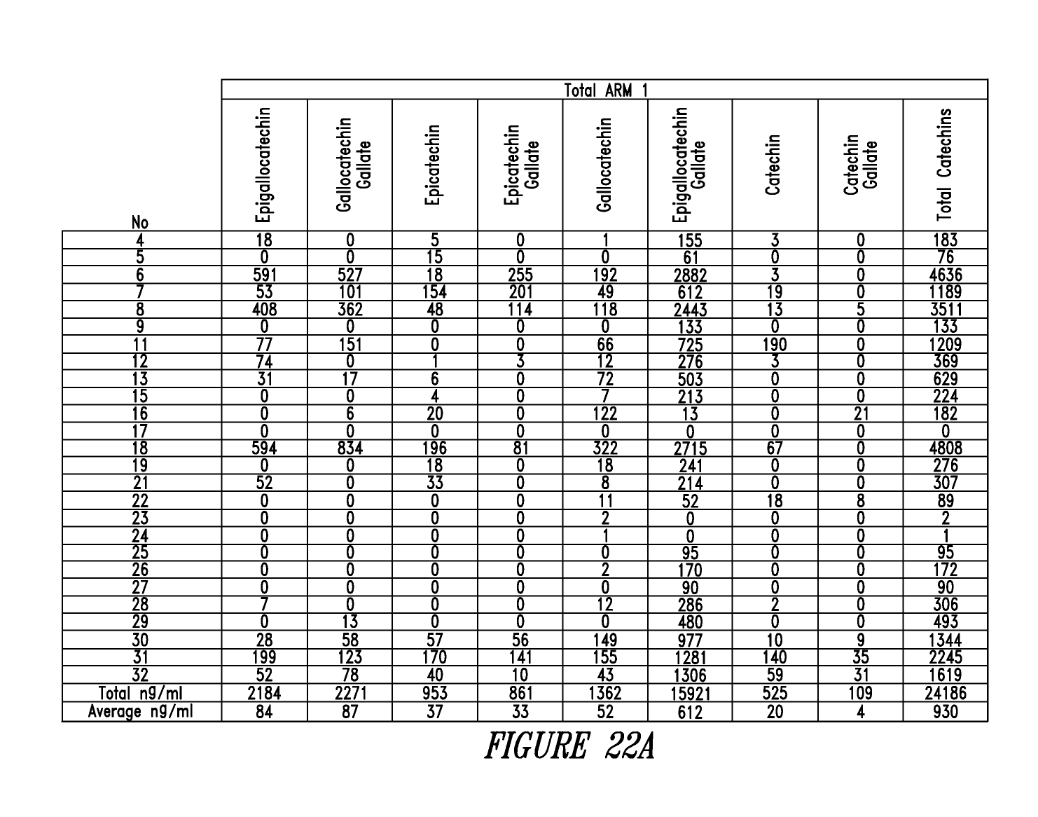

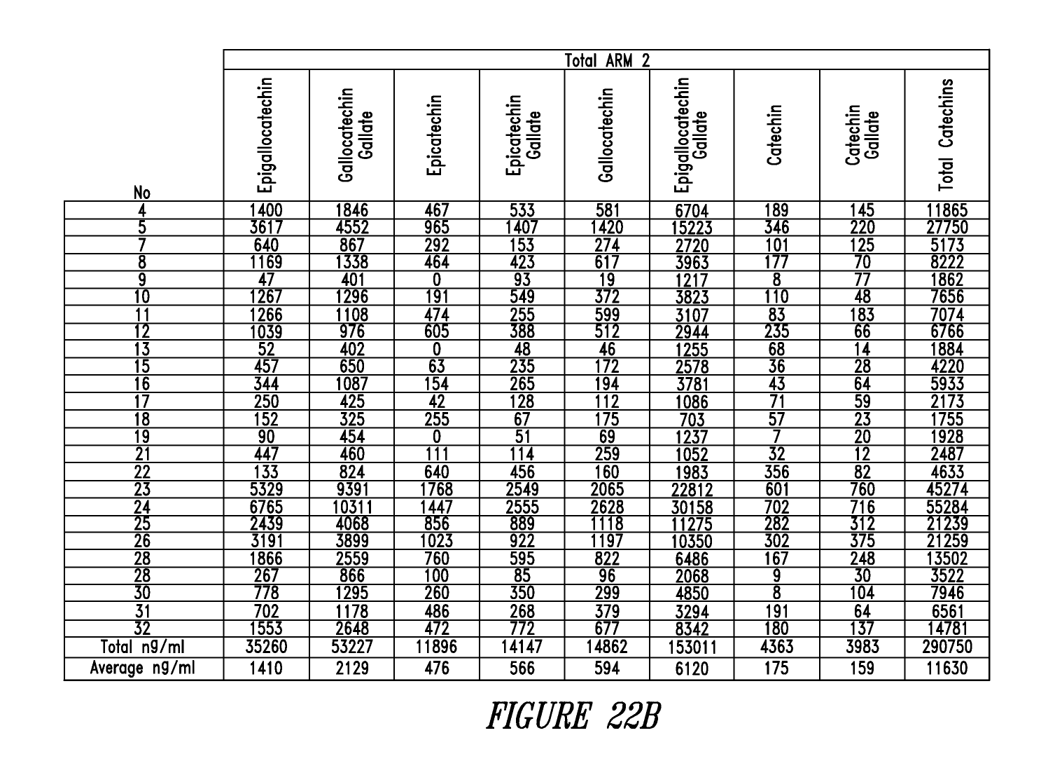

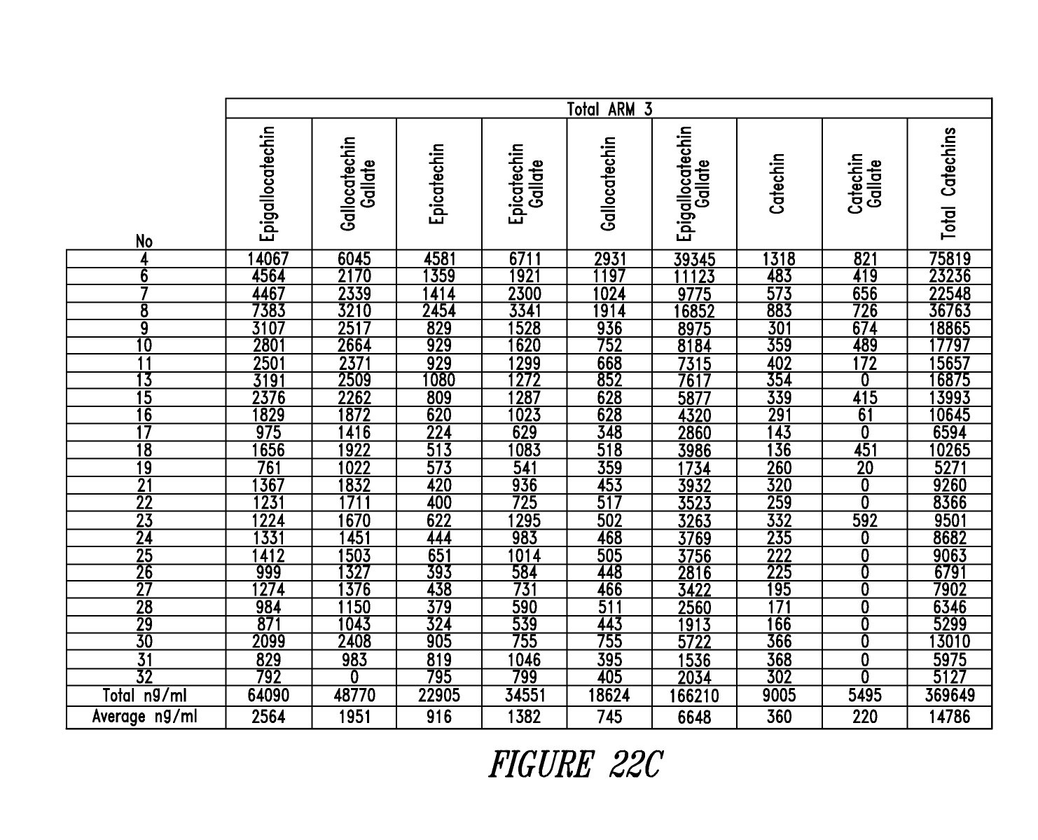

FIG. 21 shows a comparison of the average times after oral administration of the commercial and Phytofare® extract to reach the peak catechin concentrations. Time accounts for 1.61% of the total variance. F=3.37, Dfn=9, and Dfd=135. The P value is 0.0009. If time had no effect overall, there is a 0.093% chance of randomly observing an effect of the magnitude shown in FIG. 21 in an experiment of this size. The conclusion is that the effect is highly significant. FIGS. 22A-22C provide three tables with catechin levels resulting from the Arm 1, Arm 2 and Arm 3 oral dosing of green tea compositions as described in Example 44. As described in detail above, the three compositions were generic green tea extract (Arm 1), a Phytofare® Catechin Complex (Arm 2), and a Phytofare® Pheroid®Catechin Complex (Arm 3).

The following conclusions are drawn from the experiments performed according to Example 44:

1. The plasma levels of catechins were substantially and statistically significantly enhanced as a result of the Phytofare® green tea extraction process when compared to that observed for a commercial green tea extract.

2. The enhancement in plasma levels as a result of the Phytofare® extraction process were not equal for all the catechins analysed. The enhancement ranged from 8 times to 62 times. This large variation may in part be ascribed to the low values of catechins found in the commercial green tea extract for some of the catechins, most notably gallate catechins.

3. The catechin found at the highest concentration for both the commercial and Phytofare® product was epigallocatechin gallate (EGCG).

4. The plasma levels observed after administration of the Phytofare® product did not return to zero within 12 hours of administration. That resulted in an enhanced base line level from which the bioavailability parameters were calculated so that an overestimate of the enhancement in the bioavailability profile of the Phytofare® product is possible. However, in practice it would mean that the circulating half-life of the catechins prepared according to the Phytofare® is much longer and that a baseline level for catechins are maintained when using the current dosing intervals.

### Experimental Protocol.

Liquid chromatography (LC) conditions. An Agilent 1290 with binary pump with a column heater and CTC Pal auto sampler were used. Chromatographic separation was carried out on a C18 (Phenomenex Kinetex™ 30 mm×2.1 mm, 2.6 μm) reversed phase column with a pre-column (UHPLC C18, 2.1 mm ID) at 50 C. Auto sampler tray temperatures were maintained at 15° C. A gradient with LC/MS/MS grade Acetonitrile and 0.1% CHOOH was used at flow rate of 600 μl/min to separate the eight catechins. The injection volume was 10 μl.

MS/MS Conditions. An AB Sciex QTRAP® 4000 system with Turbo V™ source and electrospray ionization (ESI) was used in a negative ionization mode. The catechins were detected using their MRM transitions. Optimization of the signal was performed by constant injection of high concentrations of the 8 catechins and internal standard.

LC MS/MS Measurement. An LC MS/MS method for fast and simultaneous quantification of Catechin, Epicatechin, Catechin Gallate, Epicatechin Gallate, Gallocatechin, Epigallocatechin, Gallocatechin Gallate, Epigallocatechin gallate and Ethyl gallate (Internal Standard) was validated for linearity, sensitivity, accuracy, precision, selectivity, carry-over, recovery, matrix effect and stability according to the European Medicines Agency and US Food and Drug Administration guidelines for bio analytical method validation. Each analytical run consisted of nine spiked standards (C1-C9), 3 sets of QC samples (Low, Medium and High), blank and double blank samples. The developed method has proven to be very rapid and reliable, with the analysis requiring a three minute run time. No endogenous components interfering with analytes and the Internal Standards were found in the chromatograms of blank plasma samples.

Linearity and sensitivity were determined from the nine-point catechin standard calibration curve. The curve was constructed (Y-axis) using peak area ratios of chromatograms (catechin peak area/ISTD peak area) versus (X-axis) nominal concentration of catechin over the concentration range of 50 nM-10000 nM with an accuracy of ±15%. The European Medicines Agency and US Food and Drug Administration guidelines for bio analytical method validation were used to validate both the method and results. A minimum of 75% of the calibration points were used (at least 6 points). A linear regression weighting factor of 1/x best described the linearity of the calibration curve with regression coefficient (r²) >0.990 to (r²) >0.998.

The lower limit of quantification (LLOQ) was between 50 nM and 250 nM for the eight catechins and the signal was more than three times the signal of the blank sample. To determine the accuracy and precision for the catechins, QCs (High, Medium & Low) were compared to the standard calibration curve. Sets of QC samples were analyzed within the analytical run at least between every fifty samples. The criteria adapted from the European Medicines Agency and US Food and Drug Administration guidelines for bio analytical method validation indicated that at least 67% of the QC samples should have a mean accuracy of ±15% and at each concentration level a minimum of 50%. Furthermore the criteria indicated that the within-run % CV value should not exceed 20% for the QC samples.

The data are provided in detail in FIG. 22A-22C and summarized in the Table below:

| Dosage group and | EGCG, | GCG, | EGC, |
|---|---|---|---|
| amount, nanograms | nanograms | nanograms | nanograms |
| Generic green tea, | 15,921 | 2,271 | 2,184 |
| 24,286 | | | |
| Phytofare ®, 290,750 | 153,301 | 53,227 | 35,260 |
| nanograms | | | |
| Phytofare ® in | 166,210 | 48,770 | 64,090 |
| Pheroid ®, 369,649 | | | |
| nanograms | | | |

The dosage for each composition was 400 mg. Based on the data, a dosage such as 100 mg/day is suitable.

### REFERENCES FOR EXAMPLE 44

Auger C., Mullen W., Hara Y. & Crazier A. 2008. Bioavailability of polyphenon E flavan-3-ols in humans with an ileostomy. Journal of Nutrition. 138:1535S-1542S.
Beecher, G. R. 2003. Overview of dietary flavonoids: nomenclature, occurrence and intake. Journal of Nutrition. 133:3248S-3254S.
Bilbao, M. D. L. M., Lacueva, C. A., Roura, E., Jaurengui, O., Torre, C. & Raventos, R. M. L. 2007. A new LC/MS/MS rapid and sensitive method for the determination of green tea catechins and their metabolites in biological samples. Journal of Agricultural Food and Chemistry. 55, 8857-8863.
Cabrera, C., Artacho, R. & Gimenez, R. 2006. Beneficial effects of green tea-a review. Journal of American College of Nutrition. 25:79-99.
Camargo, A. E., Daguer, D. A. & Barbosa, D. S. 2006. Green tea exerts antioxidant action in vitro and its consumption increases total serum antioxidant potential in normal and dyslipidemic subjects. Nutrition Research. 26:626-631.
Chacko, S. M., Thambi, P. T., Kuttan, R. & Nishigaki, I. 2010 Beneficial effects of green tea: a literature review. Chinese Medicine. 5:13.
Chow, H. H., Cai, Y., Hakim, I. A., Crowell, J. A., Shahi, F., Brooks, C. A., Dorr, R. T., Hara, Y. & Alberts D S. 2003. Pharmacokinetics and safety of green tea polyphenols after multiple-dose administration of epigallocatechin gailate and polyphenon E in healthy individuals. Clinical Cancer Research. 9:3312-3319.
Chow, H. H., Cai, Y., Alberts, D. S., Hakim, I., Dorr, R., Shahi, F., Crowell, J. A., Yang, C. S. & Hara, Y. 2005. Effects of dosing condition on the oral bioavailability of green tea catechins after single-dose administration of Polyphenon E in healthy individuals. Clinical Cancer Research. 11:4627-4633.
Coimbra, S., Castro, E., Rocha-Pereira, P., Rebeto, I., Rocha, S. & Santos-Silva, A. 2006. The effect of green tea in oxidative stress. Clinical Nutrition. 25:790-796.
Dalluge, J. J. & Nelson, B. C. 2000. Determination of tea catechins. Journal of Chromatography A. 881:411-424.
Grobler A F. (2009). Pharmaceutical applications of Pheroid® technology. Potchefstroom. NWU. (Ph.D thesis) 670 p.
Henning, S. M., Niu. Y., Lee, N. H., Thames, G. D., Minutti, R. R., Wang, H., Go, V. L. & Heber, D. 2004. Bioavailability and antioxidant activity of tea flavanols after consumption of green tea, black tea, or a green tea extract supplement. Americal Journal of Clinical Nutrition. 80:1558-1564.
Kyle, J. A., Morrice, P. C., McNeill, G. & Duthie, G. G. 2007. Effects of infusion time and addition of milk on content and absorption of polyphenols from black tea. Journal of Agricultural and Food Chemistry. 55:4889-4894.
Lee, M, Maliakal, P., Chen, L., Meng, X., Bondoc, F. Y., Prabhu, S., Lambert, G., Mahr, S. & Yang, C. S. 2002. Pharmacokinetics of tea catechins after ingestion of green tea and (−)-epigallocatechin-3-gallate by humans: formation of different metabolites and individual variability. Cancer Epidemiology, Biomarkers and Prevention. 11:1025-1032.
Manach, C., Williamson, G., Morand, C., Scalbert, A. Remesy, C. and Jimenez, L. 2004. Polyphenols: food sources and bioavailability. The American journal of clinical nutrition. 79(5):727-747
McKay, D. L. & Blumberg, J. B. 2002. The role of tea in human health: an update. Journal of American College Nutrition. 21:1-13.
Moruisi, K. G. 2008. The effect of a fatty acid-based carrier on the bioavailability of epigallocatechin gallate. Potchefstroom. NWU. (M.Sc mini-dissertation) 10 Op.
Neilson, A. P. & Ferruzzi, M. G. 2011. Influence of formulation and processing on absorption and metabolism of flavan-3-ols from tea and cocoa. Annual Review of Food Science and Technology. 2:125-251.
Okushio, K., Matsumoto, N., Kohri, T., Suzuki, M., Nanjo, F. & Hara, Y. 1996. Absorption of tea catechins into rat portal vein. Biological and Pharmaceutical Bullitin. 19:326-329.
Pressman, A. H. & Buff, S. 1997. The complete idiot's guide to vitamins and minerals. New York: New York Alpha Books. 422 p.
Puch, F., Samson-Villeger, S., Guyonnet, D., Blachon, J. L., Rawlings, A. V. & Lassel, T. 2008. Consumption of functional fermented milk containing borage oil, green tea and vitamin E enhances skin barrier function. Experimental Dermatology. 17:668-674.
Reddy, V. C., Vidya Sagar, G. V., Sreeramulu, D., Venu, L. & Raghunath, M. 2005. Addition of milk does not alterthe antioxidant activity of black tea. Annuals of Nutrition and Metabolism. 49:189-195.
Sato, T. & Miyata, G. 2000. The nutraceutical benefit, part I: green tea. Nutrition. 16:315-317. Scalbert, A. & Williamson, G. 2000. Dietary intake and bioavailability of polyphenols. Journal of Nutrition. 130:2073S-2085S.
Stalmach, A., Mullen, W., Steiling, H., Williamson, G., Lean, M. E. & Crazier, A. 2010. Absorption, metabolism, and excretion of green tea flavan-3-ols in humans with an ileostomy. Molecular Nutrition and Food Research. 54:323-334.
Stalmach, A., Trouffland, S., Serafini, M. & Crazier, A. 2009. Absorption, metabolism and excretion of Choladi green tea flavan-3-ols by humans. Molecular Nutrition and Food Research. 53 Suppl 1:S44-S53.
van het Hof, K. H., Kivits, G. A., Weststrate, J A. & Tijburg, L. B. 1998. Bioavailability of catechins from tea: the effect of milk. European Journal of Clinical Nutrition. 52:356-359.
Weisburger, J. H. 1999. Tea and health: the underlying mechanisms. Proceedings of the Society of Expimental Biology and Medicyne. 220:271-275.
Williamson, G and Manach, C. 2005. Bioavailibility and bioefficacy of polyphenols in humans. II. Review of 93 intervention studies. The American Journal of Clinical Nutrition. 81(1):243s-255s.
Zhang, Q. Wang, W. Li, J. Chang, Y. Wang, Y. Zhang, J. Zhang, B. Gao, X. 2012. Simultaneous determination of catechin, epicatechin and epicatechin gallate in rat plasma by LC-ESI-MS/MS for pharmocokinetic studies after oral administration of Cynomorium songgaricum extract. Journal of Chromatography B. 880:168-171.

Having described the disclosure with reference to the Examples above, the following general information further defines the data and compositions disclosed herein.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the disclosure (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the disclosure and does not pose a limitation on the scope of the disclosure otherwise claimed. No language in the specification should be construed as indicating any non-claimed element essential to the practice of the disclosure.

Groupings of alternative elements or embodiments of the disclosure disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this disclosure are described herein, including the best mode known to the inventors for carrying out the disclosure. Of course, variations on these described embodiments will become apparent to those of ordinary skill in the art upon reading the foregoing description. The inventor expects skilled artisans to employ such variations as appropriate, and the inventors intend for the disclosure to be practiced otherwise than specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

Specific embodiments disclosed herein may be further limited in the claims using consisting of or and consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the disclosure so claimed are inherently or expressly described and enabled herein.

In closing, it is to be understood that the embodiments of the disclosure disclosed herein are illustrative of the principles of the present disclosure. Other modifications that may be employed are within the scope of the disclosure. Thus, by way of example, but not of limitation, alternative configurations of the present disclosure may be utilized in accordance with the teachings herein. Accordingly, the present disclosure is not limited to that precisely as shown and described.

1. A composition comprising processed extract of green tea plant material, wherein said plant material comprises at least one catechin, and wherein the half-life of said at least one catechin in blood plasma of a human following oral ingestion is increased relative to the half-life of unprocessed green tea plant material.

2. A composition comprising at least one green tea catechin in amorphous crystalline form, wherein said amorphous crystals are between 30 nm and at least 900 nm in size.

3. The composition of claim 2 wherein said composition comprises not less than 1% amorphous crystals by weight of total weight of composition.

4. A composition comprising at least one green tea catechin having increased bioavailability when administered to a mammal, said composition comprising at least one catechin selected from the group consisting of catechin (C), epicatechin (EC), gallocatechin (GC), epi-gallocatechin (EGC), catechin gallate (CG), epicatechin-3-gallate (ECG), gallocatechin gallate (GCG), and epigallocatechin-3-gallate (EGCG), wherein the bioavailability of said at least one catechin is increased.

5. The composition of claim 1, 2, 3 or 4, comprising at least two, three, four, five, six, seven, or eight of said catechins.

6. The composition of claim 1, 2, 3 or 4, wherein said plant material consists of green tea leaves.

7. The composition of claim 6 wherein said green tea leaves are from the plant Camellia Sinensis.

8. A pharmaceutical composition comprising the composition of claim 1, 2, 3 or 4 and a carrier, wherein said composition is formulated for topical application to the skin.

9. The pharmaceutical composition of claim 8 wherein said carrier comprises at least one of Cetyl Alcohol, Cremaphor RH40, Oleic Acid, Isopropyl meristat, Beeswax, Methyl Paraben, Propyl Paraben, BHA, and BHT.

10. The pharmaceutical composition of claim 9 wherein said carrier comprises Cetyl Alcohol, Cremaphor RH40, Oleic Acid, Isopropyl meristat, Beeswax, Methyl Paraben, Propyl Paraben, BHA, and BHT.

11. A pharmaceutical composition comprising the composition of claim 1, 2, 3 or 4, wherein said composition is formulated for the treatment of obesity in humans.

12. The pharmaceutical composition of claim 11 wherein said composition is administered orally.

13. A pharmaceutical composition comprising the composition of claim 1, 2, 3 or 4 wherein said composition is formulated for the treatment of malaria infection caused by a Plasmodium parasite.

14. The pharmaceutical composition of claim 13 wherein said Plasmodium is a Plasmodium species that infects humans.

15. The pharmaceutical composition of claim 14 wherein said Plasmodium species is selected from the group consisting of P. falciparum, P. vivax, P. ovale, and P. malariae.

16. The pharmaceutical composition of claim 15 wherein said one or more catechins inhibits the motility of said Plasmodium parasite; inhibits the hexose uptake of said Plasmodium parasite; inhibits adherence of Plasmodium-infected erythrocytes to endothelial cells; blocks binding of the erythrocytes to intracellular adhesion molecule 1; inhibits fatty acid biosynthesis in P. falciparum; inhibits an enoyl-acyl carrier protein reductase of said Plasmodium; inhibits motility of said Plasmodium; and/or binds to adhesion molecules on the Plasmodium surface and thereby inhibits gliding.
